(19) United States
(12) Patent Application Publication
(10) Pub. No.: US 2003/0143233 A1
(43) Pub. Date: Jul. 31, 2003

(54) Streptavidin expressed gene fusions and methods of use thereof

(75) Inventors: Stephen Charles Goshorn, Shoreline, WA (US); Scott Stoll Graves, Monroe, WA (US); Joanne Elaine Schultz, Seattle, WA (US); Yukang Lin, Kenmore, WA (US); James Allen Sanderson, Seattle, WA (US); John M. Reno, Brier, WA (US); Erica A. Dearstyne, Kenmore, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: NeoRx Corporation, Seattle, WA (US)

(21) Appl. No.: 10/244,821

(22) Filed: Sep. 16, 2002

Related U.S. Application Data
(63) Continuation-in-part of application No. 10/150,762, filed on May 17, 2002.
Application No. 10/150,762 is a continuation-in-part of application No. 10/013,173, filed on Dec. 7, 2001.
Application No. 10/013,173 is a continuation-in-part of application No. 09/589,870, filed on Jun. 5, 2000.
(60) Provisional application No. 60/168,976, filed on Dec. 3, 1999.
(60) Provisional application No. 60/137,900, filed on Jun. 7, 1999.

(51) Int. Cl. A61K 39/395; A61K 31/7072; A61K 31/337; C07K 16/30

(52) U.S. Cl. 424/155.1; 424/178.1; 530/388.8; 530/391.1; 514/449; 514/49

(57) ABSTRACT

The present invention provides vectors for expressing genomic streptavidin fusion cassettes. In the various embodiments, fusion proteins produced from these vectors are provided. In particular embodiments, fusion proteins comprising a single chain antibody and genomic streptavidin are provided as are vectors encoding the same. Also provided, are methods of using the fusion proteins of the present invention, in the absence and presence of a radiation-sensitizing agent, and in particular, the use of scFvSA fusion proteins as diagnostic markers or as a cell specific targeting agents.

### BACKGROUND OF THE INVENTION

1. Field of the Invention

The present invention relates generally to streptavidin expressed gene fusion constructs, and more particularly, to genomic streptavidin expressed gene fusions and methods of using these constructs in diagnostic and therapeutic applications.

1. Description of the Related Art

Streptavidin (“SA”) is a 159 amino acid protein produced by Streptomyces avidinii, and which specifically binds water-soluble biotin (Chaiet et al., Arch. Biochem. Biophys. 106:1-5, 1964). Streptavidin is a nearly neutral 64,000 dalton tetrameric protein. Accordingly, it consists of four identical subunits each having an approximate molecular mass of 16,000 daltons (Sano and Cantor, Proc. Natl. Acad. Sci. USA 87:142-146, 1990). Streptavidin shares some common characteristics with avidin, such as molecular weight, subunit composition, and capacity to bind biotin with high affinity (K_{D}≈10⁻¹⁵) (Green, Adv. Prot. Chem. 29:85-133, 1975). Further, while streptavidin and avidin differ in their amino acid compositions, both have an unusually high content of threonine and tryptophan. In addition, streptavidin differs from avidin in that it is much more specific for biotin at physiological pH, likely due to the absence of carbohydrates on streptavidin. Various comparative properties and isolation of avidin and streptavidin are described by Green et al., Methods in Enzymology 184:51-67, 1990 and Bayer et al., Methods in Enzymology 184:80-89, 1990.

The streptavidin gene has been cloned and expressed in E. coli (Sano and Cantor, Proc. Natl. Acad. Sci. USA 87(1):142-146, 1990; Agarana, et al., Nucleic Acids Res. 14(4):1871-1882, 1986). Fusion constructs of streptavidin, and truncated forms thereof, with various proteins, including single-chain antibodies, have also been expressed in E. coli (Sano and Cantor, Biotechnology (NY) 9(12):1378-1381, 1991; Sano and Cantor, Biochem. Biophys. Res. Commun. 176(2):571-577, 1991; Sano, et al., Proc. Natl. Acad. Sci. USA 89(5):1534-1538, 1992; Walsh and Swaisgood, Biotech. Bioeng. 44:1348-1354, 1994; Le, et al., Enzyme Microb. Technol. 16(6):496-500, 1994; Dubel, et al., J. Immunol. Methods 178(2):201-209, 1995; Kipriyanov, et al., Hum. Antibodies Hybridomas 6(3):93-101, 1995; Kipriyanov, et al., Protein Eng. 9(2):203-211, 1996; Ohno, et al., Biochem. Mol. Med. 58(2):227-233,1996; Ohno and Meruelo, DNA Cell Biol. 15(5):401-406, 1996; Pearce, et al. Biochem. Mol. Biol. Int. 42(6):1179-1188, 1997; Koo, et al., Applied Environ. Microbiol. 64(7):2497-2502, 1998) and in other organisms (Karp, et al., Biotechniques 20(3):452-459, 1996). Sano and Cantor (PNAS, supra) found that expression of full-length forms of streptavidin was lethal to E. coli host cells and, when capable of being expressed in truncated forms (e.g., under a T7 promoter system), only poor and varied expression was observed and the protein remained in inclusion bodies. However, there are also published reports of the expression of soluble streptavidin in E. coli (Gallizia et al., Protein Expr. Purif. 14(2):192-196, 1998; Veiko et al., Bioorg. Khim. 25(3):184-188, 1999). Those of skill in the art have frequently used “core streptavidin” (residues 14-136), or similar truncated forms, in the preparation of fusion constructs. The basis of the use of core residues 14-136 has been the observation that streptavidin preparations purified from the culture medium of S. avidinii have usually undergone proteolysis at both the N- and C-termini to produce this core structure, or functional forms thereof (Argarana et al., supra).

Presently, preparations of streptavidin expressed gene fusions are usually made by expressing a core streptavidin-containing construct in bacteria, wherein inclusion bodies are formed. Such production has several disadvantages, including the rigor and expense of purifying from inclusion bodies, the necessity of using harsh denaturing agents such as guanidine hydrochloride, and the difficulty in scaling up in an economical fashion. To a lesser extent, there has also been reported periplasmic expression of core streptavidin-containing constructs in soluble form (Dubel. et al., supra).

Therefore, there exists a need in the art for easy, cost effective, and scaleable methods for the production of streptavidin fusion proteins. Accordingly, the present invention provides several key advantages. For example, in one embodiment, a genomic streptavidin expressed gene fusion is expressed as a soluble protein into the periplasmic space of bacteria and undergoes spontaneous folding. Accordingly, such expression offers the advantage that the periplasm is a low biotin environment and one need not purify and refold the protein under harsh denaturing conditions that may prove fatal to the polypeptide encoded by a heterologous nucleic acid molecule fused to the genomic streptavidin nucleic acid molecule. The present invention fulfills this need, while further providing other related advantages.

### BRIEF SUMMARY OF THE INVENTION

The present invention generally provides expression cassettes and fusion constructs encoded thereby comprising genomic streptavidin. In one aspect, the present invention provides a vector construct for the expression of streptavidin fusion proteins, comprising a first nucleic acid sequence encoding at least 129 amino acids of streptavidin (FIG. 4), or a functional variant thereof, a promoter operatively linked to the first nucleic acid sequence, and a cloning site for, or with, insertion of a second nucleic acid sequence encoding a polypeptide to be fused with streptavidin, interposed between the promoter and the first nucleic acid sequence. Alternatively, the second nucleic acid may encode the streptavidin portion of the construct and the first nucleic acid encodes a polypeptide to be fused with streptavidin.

In certain embodiments, the promoter is inducible or constitutive. In other embodiments, the first nucleic acid sequence encodes at least amino acids 14 to 150, 14 to 151, 14 to 152, 14 to 153, 14 to 154, 14 to 155, 14 to 156, 14 to 157, 14 to 158, or 14 to 159 of streptavidin, FIG. 4, including all integer values within these ranges. In yet other embodiments, the first nucleic acid sequence encodes at least amino acids 5 to 150-159 of FIG. 4 or 1 to 150-159 of FIG. 4, including all integer values within these ranges.

Host cells containing genomic streptavidin expression cassettes are also provided as are fusion proteins expressed by the same. In certain embodiments fusion proteins comprising single chain antibodies are provided. In yet other embodiments the single chain antibodies are directed to a cell surface antigen. In yet other embodiments the single chain antibodies are directed to cell surface antigens, or cell-associated stromal or matrix antigens, including, but not limited to, CD20, CD22, CD25, CD45, CD52, CD56, CD57, EGP40 (or EPCAM or KSA), NCAM, CEA, TAG-72, γ-glutamyl transferase (GGT), mucins (MUC-1 through MUC-7), β-HCG, EGF receptor, IL-2 receptor, her2/neu, Lewis Y, GD2, GM2, tenascin, sialylated tenascin, somatostatin, activated tumor stromal antigen, or neoangiogenic antigens.

In other aspects of the present invention, methods for targeting a tumor cell are provided, comprising the administration of a fusion protein, said fusion protein comprising at least a first and a second polypeptide joined end to end, wherein said first polypeptide comprises at least 129 amino acids of streptavidin (FIG. 4), or conservatively substituted variants thereof, wherein said second polypeptide is a polypeptide which binds a cell surface protein on a tumor cell, wherein the fusion protein binds the cell surface protein on a tumor cell and wherein the streptavidin portion of the fusion protein is capable of binding biotin. In certain embodiments, the second polypeptide is an antibody or antigen-binding fragment thereof. In yet further embodiments the at least 129 amino acids comprises “core streptavidin”.

In other aspects of the present invention, pharmaceutical compositions, comprising genomic streptavidin fusion constructs are provided.

These and other aspects of the present invention will become evident upon reference to the following detailed description and attached drawings.

### BRIEF DESCRIPTION OF THE DRAWINGS

FIG. 1 is a schematic representation of a heterologous protein-genomic streptavidin expressed gene construct.

FIG. 2 is a schematic representation of a single chain antibody-genomic streptavidin fusion construct.

FIG. 3 is a schematic representation of the pEX94B expression vector containing a single chain antibody (huNR-LU-10)-genomic streptavidin fusion construct.

FIG. 4 is the sequence of genomic streptavidin (SEQ ID NO: 1) including the signal sequence and predicted amino acid sequence (SEQ ID NO: 2).

FIG. 5 is a schematic representation of the construction of the pKKlac/pelB vector.

FIG. 6 is a schematic representation of the construction of the pEX-1 vector.

FIG. 7 is a schematic representation of the construction of the pEX-SA318 and pEX-scFv3.2.1 vectors.

FIG. 8 is a schematic representation of the construction of the pEX94B vector.

FIG. 9 is a schematic representation of the construction of the pEX94B neo vector.

FIGS. 10A-10B represent the determined nucleic acid sequence (SEQ ID NO: 3) and predicted amino acid sequence (SEQ ID NO: 4) for the huNR-LU-10 single chain antibody-genomic streptavidin fusion. The streptavidin regulatory region, signal sequence, and coding sequence are noted as are the various linkers and light and heavy chains of the single chain antibody.

FIGS. 11A and 11B are the determined nucleic acid (SEQ ID NO: 5) and predicted amino acid sequences (SEQ ID NO: 6) of a B9E9 scFvSA fusion construct, with the pKOD linker between V_{L} and V_{H}. Linkers are boxed and the orientation is V_{L}-linker-V_{H}-linker-Streptavidin.

FIGS. 11C-11D are an expression cassette comprising the nucleic acid sequences (SEQ ID NO: 7) and predicted amino acid sequences (SEQ ID NO: 8) of a B9E9 scFvSA fusion construct encoding V_{H}-linker-V_{L}-linker-Streptavidin.

FIG. 12 is a scanned image representing SDS-PAGE analysis of huNR-LU-10 scFvSA.

FIG. 13 is a graphic representation of size exclusion HPLC analysis of huNR-LU-10 scFvSA.

FIG. 14 is a plot illustrating a competitive immunoreactivity assay of huNR-LU-10 scFvSA (97−20.0 and 98−01.0) as compared to huNR-LU-10 mAb.

FIG. 15 is a plot illustrating the rate of dissociation of DOTA-biotin from huNR-LU-10 scFvSA (97−13.0) as compared to recombinant streptavidin (r-SA).

FIG. 16 is a graph illustrating biodistribution of pretargeted huNR-LU-10 scFvSA.

FIG. 17 is a graph depicting blood clearance and tumor uptake of huNR-LU-10 scFvSA versus a chemically conjugated form (mAb/SA).

FIG. 18 is a bar graph illustrating biodistribution of pretargeted B9E9 scFvSA.

FIG. 19 is a scanned image of SDS-PAGE analysis of scFvSA fusion protein expression in the presence and absence of FkpA.

FIG. 20 is a schematic representation of a CC49 single chain antibody scFvSA fusion.

FIG. 21 is a schematic representation of the construction of the F5-7 CC49 expression plasmid.

FIG. 22 represents the determined nucleic acid sequence (SEQ ID NO: 48) and predicted amino acid sequence (SEQ ID NO: 49) for the CC49 single chain antibody-genomic streptavidin fusion. The streptavidin regulatory region, signal sequence, and coding sequence are noted as are the various linkers and light and heavy chains of the single chain antibody.

FIG. 23 is a graphical representation of size exclusion HPLC analysis of CC49 scFvSA.

FIG. 24 is a scanned image representing SDS-PAGE analysis of CC49 scFvSA.

FIG. 25 is a graphical representation of liquid chromatography/electrospray mass spectrometry of CC49 scFvSA.

FIG. 26 is a plot illustrating a competitive immunoreactivity assay of CC49 scFvSA and B9E9 scFvSA.

FIG. 27 is a plot illustrating the rate of dissociation of biotin from CC49 scFvSA as compared to recombinant streptavidin (r-SA).

FIG. 28 is a plot demonstrating blood clearance of CC49 scFvSA with and without addition of a clearing agent.

FIG. 29 is a bar graph illustrating biodistribution of radiolabeled CC49 scFvSA in a pretargeting regimen. Times are post administration of fusion construct.

FIG. 30 is a bar graph illustrating biodistribution of pretargeted CC49 scFvSA as measured by radiolabeled DOTA-biotin binding. Times are post administration of fusion construct.

FIG. 31 is a schematic representation of the construction of the anti-CD25 (Anti-TAC) scFvSA expression plasmid.

FIG. 32 is a bar graph illustrating biodistribution of pretargeted Anti-CD25 (anti-TAC) scFvSA as measured by radiolabeled DOTA-biotin binding. Time points are post-administration of the radiolabeled DOTA-biotin.

FIG. 33 is a graph illustrating the effect of a two-dose regimen of Gemcitabine administration with and without Pretarget radioimmunotherapy on tumor growth in nude mice.

FIG. 34 represents the determined nucleic acid sequence (SEQ ID NO: 87) and predicted amino acid sequence (SEQ ID NO: 88) for the CC49 single chain antibody-genomic streptavidin fusion. The streptavidin regulatory region, signal sequence, and coding sequence are noted as are the various linkers and light and heavy chains of the single chain antibody.

### DETAILED DESCRIPTION OF THE INVENTION

Prior to setting forth the invention, it may be helpful to an understanding thereof to set forth definitions of certain terms that will be used hereinafter.

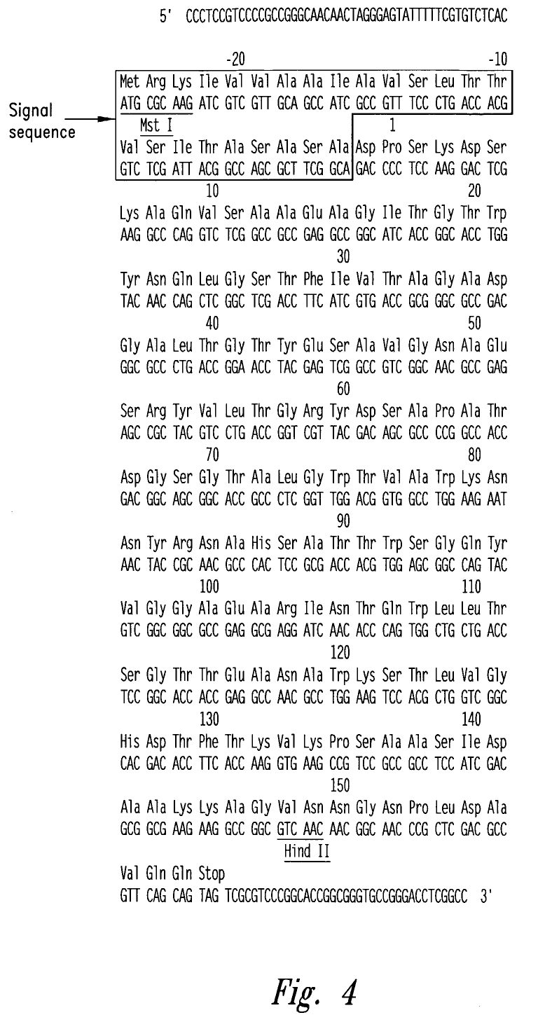

“Core streptavidin,” as used herein, refers to a streptavidin molecule consisting of the central amino acid residues 14-136 of streptavidin of FIG. 4 and also of FIG. 3 of U.S. Pat. No. 4,839,293 and deposited at ATCC number X03591, as well as that disclosed by U.S. Pat. Nos. 5,272,254 and 5,168,049 (all incorporated herein by reference).

“Genomic streptavidin,” as used herein, refers to a sequence comprising at least 129 residues of the sequence set forth in FIG. 4, wherein the at least 129 residues contains the core streptavidin sequence therein. Accordingly, genomic streptavidin refers to core streptavidin molecules comprising N-terminal, C-terminal, or both N- and C-terminal extensions. The N- and C-terminal extensions may comprise any number of amino acids selected from residues 1 to 13, 137 to 159 and all integer values between these numbers, and in some cases any number of the amino acids −1 to −24 of FIG. 4, such as −5 to −24 and any integer values therebetween.

The genomic streptavidin molecules of the subject invention also include variants (including alleles) of the native protein sequence. Briefly, such variants may result from natural polymorphisms or may be synthesized by recombinant DNA methodology, and differ from wild-type protein by one or more amino acid substitutions, insertions, deletions, or the like. Variants generally have at least 75% nucleotide identity to native sequence, preferably at least 80%-85%, and most preferably at least 90% nucleotide identity. Typically, when engineered, amino acid substitutions will be conservative, i.e., substitution of amino acids within groups of polar, non-polar, aromatic, charged, etc. amino acids. With respect to homology to the native sequence, variants should preferably have at least 90% amino acid sequence identity, and within certain embodiments, greater than 92%, 95%, or 97% identity. Such amino acid sequence identity may be determined by standard methodologies, including use of the National Center for Biotechnology Information BLAST search methodology available at www.ncbi.nlm.nih.gov using default parameters. The identity methodologies most preferred are those described in U.S. Pat. No. 5,691,179 and Altschul et al., Nucleic Acids Res. 25:3389-3402, 1997.

As will be appreciated by those skilled in the art, a nucleotide sequence and the encoded genomic streptavidin or variant thereof may differ from known native sequence, due to codon degeneracies, nucleotide polymorphisms, or amino acid differences. In certain embodiments, variants will preferably hybridize to the native nucleotide sequence at conditions of normal stringency, which is approximately 25-30° C. below Tm of the native duplex (e.g., 5×SSPE, 0.5% SDS, 5× Denhardt's solution, 50% formamide, at 42° C. or equivalent conditions; see generally, Sambrook et al., Molecular Cloning: A Laboratory Manual, 2nd ed., Cold Spring Harbor Press, 1989; Ausubel et al., Current Protocols in Molecular Biology, Greene Publishing, 1995). By way of comparison, low stringency hybridizations utilize conditions approximately 40° C. below Tm, and high stringency hybridizations utilize conditions approximately 10° C. below Tm.

A “polypeptide,” as used herein, refers to a series of amino acids of five or more.

An “isolated nucleic acid molecule” refers to a polynucleotide molecule in the form of a separate fragment or as a component of a larger nucleic acid construct, that has been separated from its source cell (including the chromosome it normally resides in) at least once, and preferably in a substantially pure form. Nucleic acid molecules may be comprised of a wide variety of nucleotides, including DNA, RNA, nucleotide analogues, or combination thereof.

The term “heterologous nucleic acid sequence”, as used herein, refers to at least one structural gene operably associated with a regulatory sequence such as a promoter. The nucleic acid sequence originates in a foreign species, or, in the same species if substantially modified from its original form. For example, the term “heterologous nucleic acid sequence” includes a nucleic acid originating in the same species, where such sequence is operably associated with a promoter that differs from the natural or wild-type promoter.

An “antibody,” as used herein, includes both polyclonal and monoclonal antibodies; humanized; Primatized® (i.e., Macaque variable region fused to human constant domains; murine; mouse-human; human-primate; mouse-primate; and chimeric; and may be an intact molecule, a fragment thereof (such as scFv, Fv, Fd, Fab, Fab′ and F(ab)′₂ fragments), or multimers or aggregates of intact molecules and/or fragments; and may occur in nature or be produced, e.g., by immunization, synthesis or genetic engineering; an “antibody fragment,” as used herein, refers to fragments, derived from or related to an antibody, which bind antigen and which in some embodiments may be derivatized to exhibit structural features that facilitate clearance and uptake, e.g., by the incorporation of galactose residues. This includes, e.g., F(ab), F(ab)′₂, scFv, light chain variable region (V_{L}), heavy chain variable region (V_{H}), and combinations thereof.

A molecule/polypeptide is said to “specifically bind” to a particular polypeptide (e.g., antibody-ligand binding) if it binds at a detectable level with the particular polypeptide, but does not bind significantly with another polypeptide containing an unrelated sequence, such that one of skill in the art would recognize as not substantially cross-reactive with the other polypeptide/molecule. An “unrelated sequence,” as used herein, refers to a sequence that is at most 10% identical to a reference sequence. In certain embodiments the binding affinity for the target will be at least 10⁻⁶ M, 10⁻⁷M, or at least 10⁻⁸M.

The term “protein,” as used herein, includes proteins, polypeptides and peptides; and may be an intact molecule, a fragment thereof, or multimers or aggregates of intact molecules and/or fragments; and may occur in nature or be produced, e.g., by synthesis (including chemical and/or enzymatic) or genetic engineering.

A radiation sensitizing agent, as used herein, refers to an agent which, when administered prior to, concurrently, or following treatment with a radioimmunotherapeutic composition, potentiates, enhances or otherwise intensifies the radiation-induced damage to a tissue and/or cellular target of the radioimmunotherapeutic composition, compared to the radiation-induced damage incurred when the radioimmunotherapeutic composition is administered to a subject in the absence of the radiation sensitizing agent, and thereby providing an increased therapeutic benefit to the subject. Although, the mechanism of action of a radiation-sensitizing agent may vary, the skilled artisan would readily appreciate that such agents include but are not limited to, for example, Gemcitabine, paclitaxel, cisplatinin and 5-fluorouracil.

A. Streptavidin Genes and Gene Products

1. Streptavidin Nucleic Acid Molecules and Variants Thereof

The present invention provides streptavidin fusion constructs that include streptavidin nucleic acid molecules of various lengths, which, in certain embodiments, are constructed from full-length genomic streptavidin nucleic acid molecules available in the art and specifically described in U.S. Pat. Nos. 4,839,293; 5,272,254, 5,168,049 and ATCC Accession number X03591.

Variants of streptavidin nucleic acid molecules, provided herein, may be engineered from natural variants (e.g., polymorphisms, splice variants, mutants), synthesized or constructed. Many methods have been developed for generating mutants (see, generally, Sambrook et al., Molecular Cloning: A Laboratory Manual, Cold Spring Harbor Press, 1989, and Ausubel, et al. Current Protocols in Molecular Biology, Greene Publishing Associates and Wiley-Interscience, New York, 1995). Briefly, preferred methods for generating nucleotide substitutions utilize an oligonucleotide that spans the base or bases to be mutated and contains the mutated base or bases. The oligonucleotide is hybridized to complementary single stranded nucleic acid and second strand synthesis is primed from the oligonucleotide. The double-stranded nucleic acid is prepared for transformation into host cells, typically E. coli, but alternatively, other prokaryotes, yeast or other eukaryotes. Standard methods of screening and isolation and sequencing of DNA were used to identify mutant sequences.

Similarly, deletions and/or insertions of the streptavidin nucleic acid molecule may be constructed by any of a variety of known methods as discussed, supra. For example, the nucleic acid molecule can be digested with restriction enzymes and religated, thereby deleting or religating a sequence with additional sequences (e.g., linkers), such that an insertion or large substitution is made. Other means of generating variant sequences may be employed using methods known in the art, for example those described in Sambrook et al., supra; Ausubel et al., supra. Verification of variant sequences is typically accomplished by restriction enzyme mapping, sequence analysis, or probe hybridization. In certain aspects, variants of streptavidin nucleic acid molecules, whose encoded product is capable of binding biotin, are useful in the context of the subject invention. In other aspects, the ability of the variant streptavidin to bind biotin may be increased, decreased or substantially similar to that of native streptavidin. In yet other embodiments, the ability to bind biotin is not required, provided that the variant form retains the ability to self-assemble into a typical tetrameric structure similar to that of native streptavidin. Such tetrameric structures have a variety of uses such as the formation of tetravalent antibodies when fused to sequences encoding an antibody or fragment thereof.

2. Genomic Streptavidin and Expression Cassettes Containing the Same

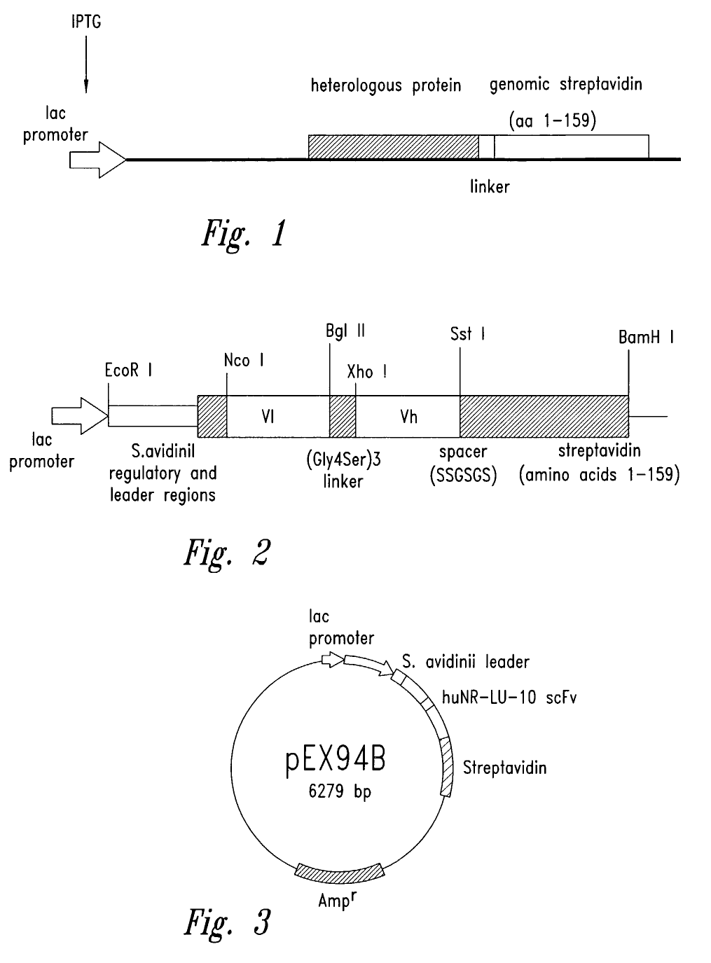

A genomic streptavidin fusion construct expression cassette of the present invention may be generated by utilizing the full gene sequence of the streptavidin gene, or a variant thereof. In certain embodiments, the expression cassette contains a nucleic acid sequence encoding at least 129 contiguous amino acids of and including at least residues 14-136 of FIG. 4 or functional variants thereof. In various other embodiments, the nucleic acid sequence encodes at least amino acid residues 14 to 140 of FIG. 4. In a further embodiment, the nucleic acid sequence encodes at least amino acids 14 to 150, 14 to 151, 14 to 152, 14 to 153, 14 to 154, 14 to 155, 14 to 156, 14 to 157, 14 to 158, or 14 to 159 of streptavidin, FIG. 4. In yet other embodiments, the nucleic acid sequence encodes at least amino acids 10 to 150-158 of FIG. 4, or 5 to 150-158 of FIG. 4 or 1 to 150-158 of FIG. 4. In yet other embodiments, the nucleic acid sequence encodes at least amino acid residues 1 to 159 of FIG. 4. In still yet other embodiments, the expression cassette comprises a nucleic acid sequence that encodes genomic streptavidin and at least 10 contiguous amino acids of residues selected from those set forth −1 to −24 of FIG. 4, such as −1 to −10, −1 to −15, −1 to −20, −5 to −15, −5 to −20, −5 to −24, or any integer value between these numbers.

As noted above, the genomic streptavidin encoding nucleic acid molecules of the subject invention may be constructed from available streptavidin sequences by a variety of methods known in the art. A preferred method is amplification (e.g., polymerase chain reaction (PCR)) to selectively amplify the individual regions and place these in cloning vectors such as pCR2.1 (Invitrogen). Moreover, such PCR reactions can be performed in a variety of ways such that the primers used for amplification contain specific restriction endonuclease sites to facilitate insertion into a vector.

Further, a variety of other methodologies besides PCR may be used to attain the desired construct. For example, one skilled in the art may employ isothermal methods to amplify the nucleotide sequence of interest, using existing restriction endonuclease sites present in the nucleotide sequence to excise and insert sequences, or by the introduction of distinct restriction endonuclease sites by site-directed mutagenesis followed by excision and insertion. These and other methods are described in Sambrook et al., supra; Ausubel, et al., supra. Briefly, one methodology is to generate single-stranded streptavidin encoding DNA, followed by annealing a primer, which is complementary except for the desired alteration (e.g., a small insertion, deletion, or mutation such that a unique restriction site is created between the domains). Bacterial cells are transformed and screened for those cells which contain the desired construct. This construct is then digested to liberate the desired sequences, which can then be purified and religated into the appropriate orientation.

One of skill in the art would recognize that the absolute length of the genomic streptavidin is only a secondary consideration when designing an expression cassette, as compared to utilizing a form which is capable of binding biotin, if so desired, and capable of expressing into the periplasmic space of a bacterial host. In certain embodiments, the expressed genomic streptavidin polypeptide fusion is present within the periplasm in a statistically significant amount as compared to heterologous fusions to core streptavidin. For any particular fusion construct of the present invention, increased localization to the periplasmic space refers in certain embodiments to the percentage of total expressed polypeptide in the periplasmic space that is at least two-fold greater than the percentage of total expressed core fusion proteins in the periplasmic space. Further, such constructs can be readily tested for their ability to bind biotin and maintain solubility in the periplasmic space by assays known in the art and those described herein. Accordingly, experiments such as, measuring biotin binding capacity and biotin dissociation rate are well known in the art and applicable in this regard. Briefly, such constructs can be tested for their ability to bind biotin by a variety of means, including labeling the fusion protein with a subsaturating level of radiolabeled biotin, then adding a 100-fold saturating level of biocytin to initiate dissociation. The free radiolabeled biotin is measured at timed intervals.

B. Vectors, Host Cells and Methods of Expressing and Producing Protein

The expression cassette of the present invention need not necessarily contain a promoter, but upon insertion into a vector system the sequence contained within the cassette must be capable of being expressed once associated with a promoter or other regulatory sequences. In one embodiment, the expression cassette itself comprises a promoter. Further, the cassette preferably contains a cloning site for the insertion of a heterologous nucleic acid sequence to be fused/linked to the genomic streptavidin encoding sequence. One exemplary cassette is set forth in FIG. 1. However, it should be noted that the cloning site need not be 5′ of the genomic streptavidin sequence, but could be placed 3′ of the streptavidin sequence. Thus, an encoded fusion protein could contain the genomic streptavidin polypeptide either N- or C-terminal to the encoded polypeptide fused thereto. Further, while it should be noted that a variety of other nucleic acid sequences can be linked to the genomic streptavidin encoding sequence, in one embodiment the sequence encodes an antibody fragment, and in certain embodiments a single chain antibody (scFv).

In addition to a cloning site, the cassette may include a linker molecule. Linker molecules are typically utilized within the context of fusion proteins and are well known in the art. As exemplified in FIG. 2, linkers are typically utilized to separate the genomic streptavidin sequence from the other sequences linked thereto and to separate the V_{H} and the V_{L} of the scFv. The linking sequence can encode a short peptide or can encode a longer polypeptide. Preferable linker sequences encode at least two amino acids, but may encode as many amino acids as desired as long as functional activity is retained. Such retained activity may include the ability to bind biotin, increased expression into the periplasmic space, or the ability of a fused antibody, antibody derived domain or fragment, to specifically bind it antigen. In the various embodiments, the linker sequence encodes 5, 6, 7, 8, 9, 10, 11, 12, 13, 14, 15, 16, 17, 18, 19, 20, 21, 22, 23, 24, 25, and 35 amino acids. In certain embodiments an encoded linker may be a standard linker such as (Gly₄Ser)ₓ (SEQ ID NO: 47) where x may be any integer, but is preferably 1 to 10. The length and composition can be empirically determined to give the optimum expression and biochemical characteristics. For example, the composition of the linker can be changed to raise or lower the isoelectric point of the molecule. Additionally, one of ordinary skill in the art will appreciate that the length of linker between variable light and heavy chains need be at least long enough to facilitate association between the two domains, while the linker between streptavidin and the antibody fragment may vary from zero amino acids to 100 or more as long as functionality is maintained. Accordingly, the linker between the light and heavy chain is typically greater than five amino acids, and preferably greater than ten, and more preferably greater than fifteen amino acids in length.

The expression cassette may be used in a vector to direct expression in a variety of host organisms. In certain embodiments, the genomic streptavidin expressed gene fusion is produced in bacteria, such as E. coli, or mammalian cells (e.g., CHO and COS-7), for which many expression vectors have been developed and are available. Other suitable host organisms include other bacterial species, and eukaryotes, such as yeast (e.g., Saccharomyces cerevisiae), plants, and insect cells (e.g., Sf9).

In one embodiment, a DNA sequence encoding a genomic streptavidin fusion protein is introduced into an expression vector appropriate for the host cell. As discussed above, the sequence may contain alternative codons for each amino acid with multiple codons. The alternative codons can be chosen as “optimal” for the host species. Restriction sites are typically incorporated into the primer sequences and are chosen with regard to the cloning site of the vector. If necessary, translational initiation and termination codons can be engineered into the primer sequences.

At a minimum, the vector will contain a promoter sequence. As used herein, a “promoter” refers to a nucleotide sequence that contains elements that direct the transcription of a linked gene. At a minimum, a promoter contains an RNA polymerase binding site. More typically, in eukaryotes, promoter sequences contain binding sites for other transcriptional factors that control the rate and timing of gene expression. Such sites include TATA box, CAAT box, POU box, AP1 binding site, and the like. Promoter regions may also contain enhancer elements. When a promoter is linked to a gene so as to enable transcription of the gene, it is “operatively linked.”

The expression vectors used herein include a promoter designed for expression of the proteins in a host cell (e.g., bacterial). Suitable promoters are widely available and are well known in the art. Inducible or constitutive promoters are preferred. Such promoters for expression in bacteria include promoters from the T7 phage and other phages, such as T3, T5, and SP6, and the trp, lpp, and lac operons. Hybrid promoters (see, U.S. Pat. No. 4,551,433), such as tac and trc, may also be used. Promoters for expression in eukaryotic cells include the P10 or polyhedron gene promoter of baculovirus/insect cell expression systems (see, e.g., U.S. Pat. Nos. 5,243,041, 5,242,687, 5,266,317, 4,745,051, and 5,169,784), MMTV LTR, CMV IE promoter, RSV LTR, SV40, metallothionein promoter (see, e.g., U.S. Pat. No. 4,870,009), ecdysone response element system, tetracycline-reversible silencing system (tet-on, tet-off), and the like.

The promoter controlling transcription of the genomic streptavidin fusion construct may itself be controlled by a repressor. In some systems, the promoter can be derepressed by altering the physiological conditions of the cell, for example, by the addition of a molecule that competitively binds the repressor, or by altering the temperature of the growth media. Preferred repressor proteins include, the E. coli lacI repressor responsive to IPTG induction, the temperature sensitive λcI857 repressor, and the like.

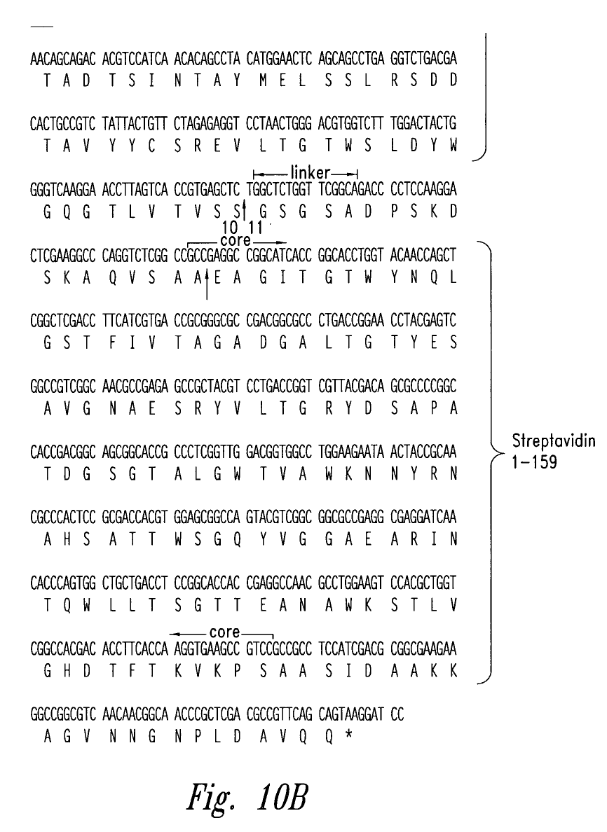

Other regulatory sequences may be included. Such sequences include a transcription termination sequence, secretion signal sequence (e.g., see FIGS. 10 and 22 as well as nucleotides 480-551 of FIG. 2B of U.S. Pat. No. 5,272,254), ribosome binding sites, origin of replication, selectable marker, and the like. The regulatory sequences are operationally associated with one another to allow transcription, translation, or to facilitate secretion. The regulatory sequences of the present invention also include the upstream region of the streptavidin gene as described in U.S. Pat. No. 5,272,254 (e.g., nucleic acid residues 174-551 depicted in FIGS. 2A-2B of U.S. Pat. No. 5,272,254). Accordingly, an upstream sequence of 100 to 300 base pairs may be utilized in expression constructs to facilitate secretion and/or expression. Such an upstream untranslated region is depicted in U.S. Pat. No. 5,272,254 FIGS. 2A and 2B as nucleotides 174-479. In preferred embodiments nucleic acid residues 408-479 of those described above are utilized in the expression construct.

In other optional embodiments, the vector also includes a transcription termination sequence. A “transcription terminator region” has either a sequence that provides a signal that terminates transcription by the polymerase that recognizes the selected promoter and/or a signal sequence for polyadenylation.

In one aspect, the vector is capable of replication in the host cells. Thus, when the host cell is a bacterium, the vector preferably contains a bacterial origin of replication. Bacterial origins of replication include the f1-ori and col E1 origins of replication, especially the ori derived from pUC plasmids. In yeast, ARS or CEN sequences can be used to assure replication. A well-used system in mammalian cells is SV40 ori.

The plasmids also preferably include at least one selectable marker that is functional in the host. A selectable marker gene includes any gene that confers a phenotype on the host that allows transformed cells to be identified and selectively grown. Suitable selectable marker genes for bacterial hosts include the ampicillin resistance gene (Amp^{r}), tetracycline resistance gene (Tc^{r}) and the kanamycin resistance gene (Kan^{r}). The ampicillin resistance and kanamycin resistance genes are presently preferred. Suitable markers for eukaryotes usually require a complementary deficiency in the host (e.g., thymidine kinase (tk) in tk-hosts). However, drug markers are also available (e.g., G418 resistance and hygromycin resistance).

The nucleotide sequence encoding the genomic streptavidin fusion protein may also include a secretion signal (e.g., a portion of the leader sequence, the leader sequence being the upstream region of a gene including a portion of a secretion signal), whereby the resulting peptide is a precursor protein processed and secreted. The resulting processed protein may be recovered from the periplasmic space or the fermentation medium. Secretion signals suitable for use are widely available and are well known in the art (von Heijne, J. Mol. Biol. 184:99-105, 1985; von Heijne, Eur. J. Biochem. 133:17-21, 1983). Prokaryotic and eukaryotic secretion signals that are functional in E. coli (or other host) may be employed. The presently preferred secretion signals include, but are not limited to, those encoded by the following bacterial genes: streptavidin, pelB (Lei et al., J. Bacteriol. 169:4379, 1987), phoA, ompA, ompT, ompF, ompC, beta-lactamase, and alkaline phosphatase.

Other components which increase expression may also be included either within the vector directing expression of the streptavidin fusion or on a separate vector. Such components include, for example, bacterial chaperone proteins such as SecA, GroEL, GroE, DnaK, CesT, SecB, FkpA, SkpA, etc.

One skilled in the art will appreciate that there are a wide variety of vectors which are suitable for expression in bacterial cells and which are readily obtainable. Vectors such as the pET series (Novagen, Madison, Wis.), the tac and trc series (Pharmacia, Uppsala, Sweden), pTTQ18 (Amersham International plc, England), pACYC 177, pGEX series, and the like are suitable for expression of a genomic streptavidin fusion protein. The choice of a host for the expression of a genomic streptavidin fusion protein is dictated in part by the vector. Commercially available vectors are paired with suitable hosts.

A wide variety of suitable vectors for expression in eukaryotic cells are also available. Such vectors include pCMVLacI, pXT1 (Stratagene Cloning Systems, La Jolla, Calif.); pCDNA series, pREP series, pEBVHis, pDisplay (Invitrogen, Carlsbad, Calif.). In certain embodiments, the genomic streptavidin fusion protein encoding nucleic acid molecule is cloned into a gene-targeting vector, such as pMC1neo, a pOG series vector (Stratagene Cloning Systems).

As noted above, preferred host cells include, by way of example, bacteria such as Escherichia coli; mammalian cells such as Chinese Hamster Ovary (CHO) cells, COS cells, myeloma cells; yeast cells such as Saccharomyces cerevisiae; insect cells such as Spodoptera frugiperda; plant cells such as maize, among other host cells.

Insect cells are capable of high expression of recombinant proteins. In this regard, baculovirus vectors, such as pBlueBac (see, e.g., U.S. Pat. Nos. 5,278,050, 5,244,805, 5,243,041, 5,242,687, 5,266,317, 4,745,051 and 5,169,784; available from Invitrogen, San Diego, Calif.) may be used for expression in insect cells, such as Spodoptera frugiperda Sf9 cells (see, U.S. Pat. No. 4,745,051). Expression in insect cells or insects is preferably effected using a recombinant baculovirus vector capable of expressing heterologous proteins under the transcriptional control of a baculovirus polyhedrin promoter. (e.g., U.S. Pat. No. 4,745,051 relating to baculovirus/insect cell expression system). Polyhedrin is a highly expressed protein, therefore its promoter provides for efficient heterologous protein production. The preferred baculovirus is Autographa californica (ACMNPV). Suitable baculovirus vectors are commercially available from Invitrogen.

Also, the fusion construct of the present invention may be expressed in transgenic animals. For example, the genomic streptavidin containing expression cassette may be operatively linked to a promoter that is specifically activated in mammary tissue such as a milk-specific promoter. Such methods are described in U.S. Pat. No. 4,873,316 and U.S. Pat. No. 5,304,498.

The genomic streptavidin gene fusion may also be expressed in plants, e.g., transgenic plants, plant tissues, plant seeds and plant cells. Such methods are described, e.g., in U.S. Pat. No. 5,202,422.

Regardless of the particular system chosen, the design of systems suitable for expression of recombinant proteins is well known and within the purview of one of ordinary skill in the art, as evidenced by the above-identified references relating to expression of recombinant fusion proteins.

Accordingly, as is evidenced by the text and examples herein, expression of fusion proteins within the context of a genomic streptavidin expressed gene fusion construct provides several key advantages. For example, in one embodiment, the genomic streptavidin fusion protein is expressed as soluble protein into the periplasmic space of bacteria (e.g., XL-1 blue, Stratagene) and undergoes spontaneous folding. Accordingly, such expression offers the advantage that the periplasm is a low biotin, oxidizing environment and produces a soluble, functional molecule. This avoids having to purify and refold the protein under harsh denaturing conditions, which may prove fatal to the polypeptide encoded by the heterologous nucleic acid molecule.

The genomic streptavidin expressed gene fusion may be isolated by a variety of methods known to those skilled in the art. However, preferably the purification method takes advantage of the presence of a functional streptavidin molecule, by utilizing its high affinity binding to aid in purification. Accordingly, preferred purification methods are by the use of iminobiotin immobilized on a solid surface.

C. Antibodies as Fusion Components

While a broad variety of genomic streptavidin expressed gene fusion molecules may be designed by the methods described herein, a particularly useful fusion protein is that of an antibody and genomic streptavidin, in particular an antibody-genomic streptavidin expressed gene fusion (Ab-SA). In preferred embodiments the expression construct encodes an Fv or scFv portion of an antibody. In a further preferred embodiment the construct encodes a Fab fragment or functional derivative thereof, to which streptavidin may be linked via a terminus of either the heavy chain portion or light chain portion of the molecule. Accordingly, DNA encoding the Fv regions of interest may be prepared by any suitable method, including, for example, amplification techniques such as polymerase chain reaction from cDNA of a hybridoma, using degenerate oligonucleotides, ligase chain reaction (LCR) (see Wu and Wallace, Genomics, 4:560, 1989, Landegren et al., Science, 241:1077, 1988 and Barringer et al., Gene, 89:117, 1990), transcription-based amplification (see Kwoh et al., Proc. Natl. Acad. Sci. USA, 86:1173, 1989), and self-sustained sequence replication (see Guatelli et al., Proc. Natl. Acad. Sci. USA, 87:1874, 1990), cloning and restriction of appropriate sequences or direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett., 22:1859-1862, 1981; and the solid support method of U.S. Pat. No. 4,458,066, as well as U.S. Pat. Nos. 5,608,039 and 5,840,300, as well as PCT Application No. WO 98/41641. DNA encoding regions of interest, for example, Fab or scFv, may also be isolated from phage display libraries.

One of ordinary skill in the art would readily recognize that given the disclosure provided herein, any number of binding pair members may be utilized and thus would not be limited to streptavidin/biotin binding. In this regard, antibody/epitope pairs or any ligand/anti-ligand pair may be utilized. One of ordinary skill in the art would also appreciate that the present disclosure provides a general method for the preparation of tetravalent antibodies. Since the avidity of an antibody for its cognate antigen is generally a function of its valency, there are many applications in which a tetravalent antibody would be preferable to a divalent antibody. Such applications include, but are not limited to, immunoassays, immunotherapy, immunoaffinity chromatography, etc.

Chemical synthesis may also be utilized to produce a single stranded oligonucleotide. This may be converted into double stranded DNA by hybridization with a complementary sequence, or by polymerization with a DNA polymerase using the single strand as a template. While it is possible to chemically synthesize an entire single chain Fv region, it is preferable to synthesize a number of shorter sequences (about 100 to 150 bases) that are later ligated together.

Alternatively, subsequences may be cloned and the appropriate subsequences cleaved using appropriate restriction enzymes. The fragments may then be ligated to produce the desired DNA sequence.

Once the variable light (V_{L}) and heavy chain (V_{H}) DNA is obtained, the sequences may be ligated together, either directly or through a DNA sequence encoding a peptide linker, using techniques well known to those of skill in the art. In a preferred embodiment, heavy and light chain regions are connected by a flexible polypeptide linker (e.g., (Gly₄Ser)ₓ, or the pKOD sequence, or others, such as those provided, infra) which starts at the carboxyl end of the light chain Fv domain and ends at the amino terminus of the heavy chain Fv domain, or vice versa, as the order of the Fv domains can be either light-heavy or heavy-light. The entire sequence encodes the Fv domain in the form of a single-chain antigen binding protein.

A variety of methods exist for the recombinant expression of immunoglobulins. The following references are representative of methods and host systems suitable for expression of recombinant immunoglobulins and fusion proteins in general: Weidle et al., Gene 51:21-29,1987; Dorai et al., J. Immunol. 13(12):4232-4241, 1987; De Waele et al., Eur. J. Biochem. 176:287-295, 1988; Colcher et al., Cancer Res. 49:1738-1745,1989; Wood et al., J. Immunol. 145(a):3011-3016, 1990; Bulens et al., Eur. J. Biochem. 195:235-242 1991; Beggington et al., Biol. Technology 10:169, 1992; King et al., Biochem. J. 281:317-323, 1992; Page et al., Biol. Technology 9:64, 1991; King et al., Biochem. J. 290:723-729, 1993; Chaudary et al., Nature 339:394-397, 1989; Jones et al., Nature 321:522-525, 1986; Morrison and Oi, Adv. Immunol. 44:65-92, 1988; Benhar et al., Proc. Natl. Acad. Sci. USA 91:12051-12055, 1994; Singer et al., J. Immunol. 150:2844-2857, 1993; Cooto et al., Hybridoma 13(3):215-219, 1994; Queen et al., Proc. Natl. Acad. Sci. USA 86:10029-10033, 1989; Caron et al., Cancer Res. 32:6761-6767, 1992; Dubel et al., J. Immunol. Methods 178:201-209, 1995; Batra et al., J. Biol. Chem. 265:15198-15202, 1990; Batra et al., Proc. Natl. Acad. Sci. USA, 86:8545-8549, 1989; Chaudhary et al., Proc. Natl. Acad. Sci. USA, 87:1066-1070, 1990, several of which describe the preparation of various single chain antibody expressed gene fusions.

Accordingly, once a DNA sequence has been identified that encodes an Fv region which when expressed shows specific binding activity, fusion proteins comprising that Fv region may be prepared by methods known to one of skill in the art. The Fv region may be fused to genomic streptavidin directly in the expression cassette of the present invention or, alternatively, may be joined directly to genomic streptavidin through a peptide or polypeptide linker, thereby forming a linked product. The linker may be present simply to provide space between the Fv and the fused genomic streptavidin or to facilitate mobility between these regions to enable them to each attain their optimum conformation. The genomic streptavidin-antibody expression cassette, typically, comprises a single vector which provides for the expression of both heavy and light variable sequences fused by an appropriate linker as well as a linker fusing the light and heavy chains with genomic streptavidin, thereby encoding a single chain antibody:genomic streptavidin (scFvSA) conjugate. In one embodiment the linker connecting the variable light and heavy chains is of sufficient length or side group selection to allow for flexibility. In one embodiment the linker is a standard linker such as (Gly₄Ser)ₓ, described supra, while in another embodiment the linker is the pKOD linker (GlyLeuGluGlySerProGluAlaGlyLeuSerProAspAlaGlySerGlySer) (SEQ ID NO: 9). It should be understood that a variety of linkers may be used, but in some embodiments it may be preferred that the linker separating the light and heavy antibody chains should allow flexibility and the linker attaching the scFv to the genomic streptavidin sequence can be fairly rigid or fairly flexible. Further, in addition to linkers, additional amino acids may be encoded by the addition of restriction sites to facilitate linker insertion and related recombinant DNA manipulation. As such, these amino acids, while not necessarily intended to be linkers, may or may not be included within the constructs described herein, depending on the construction method utilized.

Exemplary linkers are known by those of skill in the art. For example, Fv portions of the heavy and light chain of antibodies held together by a polypeptide linker can have the same binding properties as their full length two chain counterparts (Bird et al., Science, 242:423-26, 1988 and Huston et al., Proc. Natl. Acad. Sci. USA, 85:5879-83, 1988). It has also been shown that, in some cases, fusion proteins composed of single chain antibodies linked to toxins may retain the binding capacity of the single chain antibody as well as the activity of the toxin (Chaudary et al., Nature, 339: 394-97, 1989; Batra et al., J. Biol. Chem., 265: 15198-15202, 1990; Batra et al., Proc. Natl. Acad. Sci. USA 86: 8545-8549, 1989; Chaudary et al., Proc. Natl. Acad. Sci. USA 87:1066-1070, 1990). Exemplary fusion constructs containing streptavidin are described by Sheldon et al., Appl. Radiat. Isot. 43(11):1399-1402, 1992; Sano and Cantor, Bio/Technology 9:1378-1381, 1991; Spooner et al., Human Pathology 25(6):606-614, 1994; Dubel et al., J. Immun. Methods 178:201-209, 1995; Kipriyanov et al., Protein Engineering 9(2):203-211,1996. The DNA sequence comprising the linker may also provide sequences, such as primer sites or restriction sites, to facilitate cloning or may preserve the reading frame between the sequence encoding the scFv and the sequence encoding genomic streptavidin. The design of such linkers is well known to those of skill in the art.

Further, one skilled in the art would find it routine to test the ability of genomic streptavidin-antibody expressed gene fusions to bind the appropriate ligand. In contemplated embodiments, this ligand antigen may be a cell surface antigen, cell-associated stromal or matrix antigen, or cell-secreted antigens, including, but not limited to, CD19, CD20, CD22, CD25, CD33, CD45, CD52, CD56, CD57, EGP40 (or EPCAM or KSA), NCAM, CEA, TAG-72, γ-glutamyl transferase (GGT), a mucin (MUC-1 through MUC-7), β-HCG, EGF receptor and variants thereof, IL-2 receptor, her2/neu, Lewis Y, GD2, GM2, Lewis x, folate receptor, fibroblast activation protein, tenascin, sialylated tenascin, somatostatin, activated tumor stromal antigen, or a neoangiogenic antigen. Moreover, methods for evaluating the ability of antibodies to bind to epitopes of such antigens are known.

D. Applicable Uses of Genomic Streptavidin Expressed Fusion Constructs

While any heterologous nucleic acid sequence can be joined to that encoding genomic streptavidin and expressed, as described herein, particularly useful expressed fusion constructs are those comprising scFv linked, to genomic streptavidin (SA), referred to previously as scFvSA. Accordingly, in one aspect of the invention, scFv antibody and/or fragments thereof are useful as tools in methods for medical diagnostic and/or therapeutic purposes. In this context, a diagnostic or therapeutic method, as described herein, can be used for detecting the presence or absence of, or in the treatment of, a target site within a mammalian host. In some cases, the target site may constitute a tumor. In any circumstance, the skilled artisan will appreciate that when determining the criteria for employing antibodies or antibody conjugates (e.g., antibody fusion protein) for in vivo administration, for example in treating a tumor target for therapeutic or diagnostic purposes, it is desirable that the targeting ratio of the conjugate fusion protein (bound vs. unbound) is high while, at the same time, the absolute dose of therapeutic agent delivered to the tumor is sufficient to elicit a significant and/or desired tumor response. Methods for utilizing such antibodies described in the present invention can be found, for example, in U.S. Pat. Nos. 4,877,868, 5,175,343, 5,213,787, 5,120,526, and 5,200,169.

In addition, it may also be desirable to minimize exposure of non-targeted tissues to a therapeutic agent being administered, therapeutically or diagnostically. One method that can be used to reduce and/or otherwise minimize the exposure of non-targeted tissue to an administered targeted agent, diagnostic or therapeutic, may first involve “pretargeting” of the targeted agent by way of its targeting moiety (e.g., the scFv portion of an scFvSA fusion protein), to a desired target site (i.e., antigen). It is further appreciated that the administered therapeutic agent (e.g., scFvSA), is selected for its ability to be rapidly cleared. In this context, the therapeutic agent which does not bind to the target antigen (i.e., is unbound) may be cleared from circulation, if so desired, by administration of a clearing agent, thereby reducing or otherwise minimizing exposure of the targeted therapeutic agent and therapeutic compound (active agent) to non-targeted sites, which the skilled artisan will recognize as consistent with reducing non-specific background or increasing signal to noise ratio.

Following such pretargeting, a therapeutic compound (active agent) may then be administered, wherein the therapeutic compound binds to the antigen-bound pretargeted therapeutic agent by way of, for example, the SA portion of the antigen-bound pretargeted scFvSA fusion protein (conjugate), i.e., the active agent becomes scFvSA-bound.

Therefore, in this method, as it is generally described, an optional intermediate step may involve administration of a clearing agent to aid in the efficient removal of unbound targeted therapeutic agent (targeting moiety conjugate, antibody fusion protein) prior to administration of the therapeutic compound (active agent conjugate). A description of embodiments of the pretargeting technique, including the description of various clearing agents and/or chelating agent (chelators), such as DOTA, may be found in U.S. Pat. Nos. 4,863,713, 5,578,287, 5,608,060, 5,616,690, 5,630,996, 5,624,896, 5,847,121, 5,911,969, 5,914,312, 5,955,605, 5,976,535, 5,985,826, 6,015,897, 6,022,966, 6,075,010, 6,217,869, 6,287,536; and PCT publication Nos. WO 93/25240, WO 95/15978, WO 97/46098, WO 97/46099, which are incorporated herein in their entirety.

In the pretargeting approach described herein, the pharmacokinetics of the active agent is uncoupled from that of the targeting moiety (i.e., scFv) of the pretargeted therapeutic agent, fusion protein. Accordingly, in one embodiment of the present invention, scFvSA, a conjugate (fusion protein) of the targeting moiety (scfv) and ligand binding moiety, for example streptavidin (SA), is administered and allowed to accrete to a target site. After accretion occurs, fusion protein that is not associated with a target site may be cleared from the recipient's circulation either by an intrinsic clearance mechanism or via administration of a ligand or anti-ligand containing synthetic clearing agent, which may recognize either the targeting moiety or the SA moiety of the targeting agent.

After accretion of the targeting agent and, optionally, removal of non-target bound therapeutic targeting agent, an active agent (therapeutic compound) is administered which binds to or otherwise complements with, for example, the SA moiety of the scFvSA fusion protein (e.g., biotin would bind to or be considered to complement with the above-mentioned SA moiety). Preferably, the active agent (ligand binding agent or anti-ligand-agent) has a short serum half life and is excreted via the renal system if it is not associated with, for example, a targeted scFvSA conjugate fusion protein. In this manner, therefore, the therapeutically active agent either accretes to the fusion protein already bound at the target site, where its therapeutic or diagnostic functionality is desired, or it is rapidly removed from the recipient, thereby reducing or otherwise minimizing undesired toxicity to non-targeted tissues and/or cells of the recipient. One of ordinary skill in the art would further appreciate that in order to enhance renal excretion of non-bound active agent, the active agent may be conjugated to a renal excretion-promoting, biodistribution-directing (modulating) molecule.

Accordingly it is understood that the pretargeting methods described herein are characterized by an improved targeting ratio (bound vs. unbound) or an increase in the absolute amount of active agent delivered to the target sites on a cell compared to conventional cancer diagnostic methods, and/or therapy.

In one embodiment of the pretarget methodology, the targeting moiety will comprise an antibody fusion of the present invention specific for a particular antigen associated with the target cells of interest. In certain embodiments, the targeting moiety will comprise an antibody fusion comprising the CC49 antibody, or a functional homologue or fragment thereof. Such a targeting moiety should be capable of specifically binding CC49's cognate antigen, TAG-72. Specific disease states that may be targeted by such a targeting moiety include, but are not limited to, any TAG-72 positive human carcinoma or adenocarcinoma of the gastrointestinal tract (e.g., colon, rectum, gastric, esophagus), pancreas, ovary, endometrium, breast, prostate, lung, appendix, liver, salivary duct, including metastatic cancers, as well as cholangiocarcinoma.

In other embodiments, the targeting moiety will comprise an antibody fusion comprising the B9E9 antibody, or a functional homologue or fragment thereof, capable of binding its cognate antigen, CD20. Specific disease states targeted by such a targeting moiety include, but are not limited to, lymphomas, such as follicular, mantle cell, diffuse large B-cell, precursor B-lymphoblastic, lymphoplasmacytoid, marginal zone B-cell, splenic marginal zone, Burkitt, high grade B-cell, B-cell chronic lymphocytic, small lymphocytic, lymphoplasmacytoid, and plasmacytoma/melanoma, for example, as well as leukemias, such as prolymphocytic, B-cell chronic lymphocytic, precursor B-lymphoblastic, and hairy cell, for example. In addition, it is appreciated that B9E9 scFvSA is a genetic fusion of the single-chain variable region of the murine anti-CD20 antibody B9E9 to the genomic streptavidin of Streptomyces avidinii, and is a stable tetramer, consisting of 4 identical subunits containing a single chain B9E9 antibody fragment and a streptavidin subunit. The resulting species is tetravalent with respect to both antigen and biotin binding. However, an observed increased antigen-binding avidity should decrease streptavidin dissociation from tumor. In animals, the B9E9 scFvSA exhibited more rapid systemic clearance than other antibody/SA conjugates, which is consistent with its smaller size and lack of the Fc region of the antibody. The biochemical uniformity of B9E9 scFvSA alone makes it a superior agent compared with other first-generation antibody/streptavidin conjugates. Similar advantageous characteristics are present with the CC49 scFvSA as well as other antibody streptavidin fusions, for example anti-CD25 scFvSA, as disclosed below. Accordingly, the aforementioned diseases serve as appropriate clinical indications for methods of the invention, including diagnostic assays and therapeutic treatment.

In a further embodiment, the targeting moiety will comprise an antibody fusion protein comprising the anti-TAC antibody (also referred to herein as anti-CD25), or a functional homologue or fragment thereof, capable of binding its cognate antigen, CD25. Specific disease states and/or cancer indication(s) that may be targeted by such a anti-CD25 targeting moiety include, but are not be limited to, HTLY-1-associated adult T-cell leukemia (ATL), stages Ib through IV of cutaneous T-cell lymphoma (CTCL), peripheral T-cell lymphoma (PTC), prolymphocytic leukemia (PLL), Hodgkin's disease and non-Hodgkins lymphoma (NHL), wherein it is preferred that the target antigen is present on a statistically significant number of malignant cells, for example greater than 25% of malignant cells, taken from blood, lymph node or other relevant site. Accordingly, the aforementioned diseases, by way of example, as they are associated with expression of CD25 will serve as appropriate clinical indications for methods of the instant invention, including diagnostic assays and therapeutic treatment.

The Pretarget embodiments listed above, by way of example but not in limitation, including lymphoma, comprise a powerful delivery system for radioimmunotherapy, exploiting the strong affinity of streptavidin for biotin (Kd=10⁻¹⁵M). In certain embodiments of the examples provided herein, two or three steps may be utilized. The first step involves the injection of an antibody fusion protein that targets, for example, CD20, a cell-surface antigen expressed on approximately 90% of B-cell lymphomas. The fusion protein, B9E9 scFvSA, is a genetic fusion of the single-chain variable region of the murine anti-CD20 antibody B9E9 to the genomic streptavidin of Streptomyces avidinii. A similar first step may use an antibody fusion protein that targets expression of, for example, CD25, as described above. Second, optionally, after allowing accretion of peak levels of fusion protein at the target site (e.g., tumor cell), a synthetic clearing agent is injected to remove unbound fusion protein from the circulation. Finally, in step three, radiolabeled DOTA-Biotin is injected (i.e., active agent). Due to the strong affinity between streptavidin and biotin, the radiolabeled DOTA-Biotin binds to the streptavidin moiety of the pretargeted fusion protein bound to target tumor cells, while unbound radiolabeled DOTA-Biotin is rapidly excreted through the kidneys, as discussed above. Thus, a radiation treatment delivered through binding of the radiolabeled therapeutic active agent to the pretargeted scFvSA fusion protein bound to a target cell antigen is itself targeted directly to the tumor cell, with little uptake in non-targeted tissues and/or cells. Accordingly, the instant invention allows delivery of more radiation to the tumor, and improved tumor response to treatment.

Metastatic or recurrent gastrointestinal (GI) cancers represent a common and therapeutically frustrating form of cancer. They primarily represent adenocarcinomas arising from the GI tract (colorectal and gastric), pancreas and biliary tract (cholangiocarcinoma). A useful treatment modality is to target such cancer cells utilizing cell surface markers. One such marker is the TAG-72 antigen that has been used as the target in numerous radioimmunotherapy studies. The antigen, characterized as a high-molecular weight glycoprotein with mucin properties, has been purified from a human xenograft colon carcinoma designated LS-174T. TAG-72 is expressed in several epithelial-derived cancers, including most adenocarcinomas of the gastrointestinal tract, invasive ductal carcinomas of the breast, non-small cell lung carcinomas, and common epithelial ovarian carcinomas. TAG-72 expression has not been observed in tumors of neural, hematopoietic or sarcomatous derivation. Immunohistochemical studies have reported that TAG-72 is not appreciably expressed on normal tissues, with the exception of secretory endometrium and colonic epithelium. Another study showed TAG-72 reactivity in extracts of normal lung and stomach tissues by solid phase radioimmunoassay, and with small bowel, testis, lung and stomach by immunohistology. TAG-72 has previously been shown to be distinct from carcinoembryonic antigen, EpCam and other tumor-associated antigens.

Many antibodies to the TAG-72 antigen have been produced. In addition to B72.3, a murine anti-TAG-72 antibody, several second-generation antibodies have been generated using TAG-72 as the immunogen. Nine of these second generation antibodies, including CC49, have been further characterized. CC49 binds to a disaccharide epitope, designated sialyl Tn, on the TAG-72 antigen. This epitope is expressed by about 85% of human adenocarcinomas, including colon, breast, pancreatic, ovarian, endometrial, non-small cell lung and gastric cancers. In studies using human xenografts in athymic mice, CC49 had a 6-fold higher affinity constant and a 16-fold increase in the tumor:blood ratio than B72.3. The pancarcinoma distribution of the antigen and minimal reactivity of anti-TAG-72 antibodies with normal adult tissues suggest potential diagnostic and therapeutic utility for many human carcinomas.

Any other targeting antibodies may be used in the Pretarget regimen. For example, anti-TAC scFvSA may be utilized in the 3-step Pretarget regimen in the treatment of patients with CD-25-positive leukemias and lymphomas. In the first step, injected anti-TAC scFvSA binds to the tumor. After allowing fusion protein to accrete to peak levels at the tumor, a synthetic clearing agent is injected to remove unbound anti-TAC scFvSA from the circulation (step two). And finally, if the residual biotin-binding assay indicates adequate blood clearance of anti-TAC scFvSA, radiolabeled DOTA-Biotin is injected (step three).

In certain therapeutic and diagnostic embodiments, the formulation and dosing of the various components can vary, depending upon the preferred dosage level identified during the course of clinical trials. In certain embodiments, the formulation prepared may include an scFvSA fusion protein that was produced in an E. coli fermentation process, where the scFv antigen-binding portion of the antibody of interest is genetically linked to genomic streptavidin (SA). The scFvSA, although expressed as a monomer, spontaneously folds into soluble tetramers, as discussed above. The scFvSA fusion protein may then be formulated, for example, at a concentration of 5 mg/ml in phosphate buffered saline containing 5% sorbitol, or in 5 mM histidine containing 2-5% trehalose, and the resulting formulation may be lyophilized.

As discussed for the second step, the disclosed methods may include administration of a clearing agent to remove unbound targeting agent (fusion protein). For example, one such Synthetic Clearing Agent (sCA) (MW=8651.7 Daltons) that may be used is a non-toxic synthetic biotin galactosamine compound. This synthetic biotin galactosamine compound contains no acidic or basic functional residues and is uncharged at physiological pH, and may be supplied as an aseptically filled, sterile, pyrogen free solution in water at 12.7 mg sCA/ml, administered in 100 mL saline.

The third step in a Pretarget regimen, as discussed above, may be for example, administration of DOTA-Biotin (MW=807 Daltons) that is a synthetic molecule containing a biotin attached to the macrocyclic amino benzyl DOTA chelate through an N-methyl glycine. Amino benzyl DOTA is designed for stable chelation of 3+ metals such as Yttrium and Indium. It may be supplied at a concentration of 12 mg/ml. As would be appreciated by those or ordinary skill in the art, any of the formulations, or components thereof, may be prepared in lyophilized form, and rehydrated as needed.

Formulations and compositions of this invention may comprise any of the fusion proteins of the present invention, in the presence or absence of a radiation-sensitizing agent, and any physiologically acceptable carrier, adjuvant or vehicle, such as any pharmaceutically acceptable carrier.

Physiologically acceptable carriers including adjuvants and vehicles that may be used in the compositions of this invention include, but are not limited to, lecithin; serum proteins, such as human serum albumin; buffer substances such as the various phosphates, glycine, potassium sorbate, partial glyceride mixtures of saturated vegetable fatty acids; water, salts or electrolytes, such as protamine sulfate, disodium hydrogen phosphate, potassium hydrogen phosphate, and sodium chloride; colloidal silica, cellulose-based substances, polyethylene glycol, sodium carboxymethylcellulose, polyarylates, waxes, polyethylene-polyoxypropylene-block polymers, polyethylene glycol and wool fat, and the like. A thorough discussion of acceptable carriers is available in Remington's Pharmaceutical Sciences, Mack Publishing Co., NJ, 1991. Pharmaceutical compositions also are provided by the present invention.

Ordinarily, the preparation of such compositions, which may include a physiologically acceptable carrier, entails combining the therapeutic agent, for example a fusion protein, with buffers, antioxidants such as ascorbic acid, low molecular weight (less than about 10 residues) polypeptides, proteins, amino acids, carbohydrates including glucose, sucrose or sorbitol dextrins, chelating agents such as EDTA, glutathione and other stabilizers and excipients. Neutral buffered saline or saline mixed with nonspecific serum albumin are exemplary appropriate diluents.

In addition, the compositions, for example a fusion protein, and physiological acceptable carriers, including pharmaceutical compositions, of the present invention may be prepared for administration by a variety of different routes, including for example intraarticularly, intracranially, intradermally, intrahepatically, intramuscularly, intraocularly, intraperitoneally, intrathecally, intravenously, subcutaneously or even directly into a tumor. In addition, pharmaceutical compositions of the present invention may be placed within containers, along with packaging material, which provides instructions regarding the use of such pharmaceutical compositions. Compositions, for example pharmaceutical compositions, of the present invention may be administered in a manner appropriate to the disease to be treated (or prevented). The quantity and frequency of administration will be determined by such factors as the condition of the patient, and the type and severity of the patient's disease. Generally, such instructions will include a tangible expression describing the reagent concentration, as well as relative amounts of excipient ingredients or diluents (e.g., water, saline or PBS), which may be necessary to reconstitute the pharmaceutical composition. Pharmaceutical compositions are useful for both diagnostic and therapeutic purpose.

Dosing in the three step system will be determined by clinical trials but can, at least initially, be investigated utilizing the following parameters: step one, administration of fusion protein at 160-480 mg/m²; then (step two), between 20-72 hours thereafter, administration of clearing agent at 23-90 mg/m²; then (step three) administration of radiolabeled DOTA-Biotin at 0.33 to 2.6 mg/m² 10-24 hours after administration of clearing agent. In most cases a residual biotin-binding assay may be performed prior to administering radiolabeled DOTA-biotin, in order to monitor blood clearance of the fusion protein, targeting agent.

In related embodiments, the antigen marker may be associated with a cancer, including, but not limited to, the following: lymphoma (e.g., CD20); leukemia (e.g., CD25 and/or CD45); prostate (e.g., TAG-72); ovarian (e.g., TAG-72); breast (e.g., MUC-1 and/or TAG-72); colon (e.g., CEA, TAG-72); and pancreatic (e.g., TAG-72). For example, the CD20 antigen may be targeted for the treatment of lymphoma wherein the ligand/anti-ligand binding pair may be biotin/avidin (e.g., streptavidin-gene fusion (scFvSA), and the active agent will be a radionuclide in pretargeting methods. Further, a variety of antigens may be targeted, such as CD45 antigen targeting for pretargeted radioimmunotherapy (PRIT) to treat patients having any one of a broad range of hematologic malignancies by employing antibody-mediated targeting to the CD45 antigen. CD45 is the most broadly expressed of the known hematopoietic antigens, found on essentially all white blood cells and their precursors, including neutrophils, monocytes and macrophages, all lymphocytes, myeloid and lymphoid precursors, and about 90% of acute myelogenous leukemia (AML) cells. Accordingly, as the antigens available for targeting for diagnostic or therapeutic purposes are numerous, the present invention may be used to facilitate targeting to any of these antigens.

An optional step in pretarget methods, including those identified above, comprises the initial administration of a non-conjugated targeting moiety (i.e., not conjugated to a ligand binding moiety or anti-ligand) or, alternatively, administering this non-conjugated targeting moiety concurrently with the conjugated (fusion protein) form in the first step, thus blocking those targets that may be contacted initially. It is appreciated that such blocking may be especially useful, for example, in the treatment of non-Hodgkin's lymphoma, where the first set of targeted tissues may be the spleen, while most tumors are found in the deep lymph nodes. Such pre-blocking allows for substantial protection of the spleen cells from later treatment with the active agent. While the non-conjugated targeting agent need not necessarily bind the same epitope, to be effective it should preclude binding by the targeting moiety conjugate.

It is appreciated that a radiation-sensitizing agent may, but need not, be administered concurrently with administration of the radioimmunotherapeutic composition. The skilled artisan would readily understand that a radiation-sensitizing agent can be administered, in an effective manner, prior to, concurrently with, or following administration of the radioimmunotherapeutic composition. Similarly, those of ordinary skill would appreciate that a radiation-sensitizing agent can be administered at a plurality of times, for example, a first administration prior to administration of a radioimmunotherapeutic composition, and then a second administration concurrent with administration of the radioimmunotherapeutic composition. A second administration of the radiation-sensitizing agent may be after concurrent administration of the radioimmunotherapeutic composition and radiation-sensitizing agent. Variation in time and dosage delivered in such a plurality of administration schedule may be adjusted according to the particular subject or therapeutic target, for example, cancer.

One skilled in the art could use multiple targeting moiety conjugate fusion proteins comprising different antibodies that also bind to the same cell type to enhance the therapeutic effect or diagnostic utility. For example, U.S. Pat. No. 4,867,962 issued to Abrams describes such an improved method for delivering active agent to target sites, employing active agent-targeting moiety conjugates. Briefly, the Abrams method contemplates administration of two or more active agent-targeting moiety conjugates, wherein each conjugate includes a different antibody species as the targeting moiety. Each of the antibody species is reactive with a different target site epitope (associated with the same, or a different, target site antigen), while, at the same time, the patterns of cross-reactivity of the antibody species with non-target tissues are non-overlapping. In this manner the different antibodies accumulate (accrete) additively at the desired target site, while fewer than the total of both species combined accumulate at any type of non-target tissue. Thus, a higher percentage of the administered therapeutic agent becomes localized at in vivo target sites than at non-target sites. The present invention encompasses approaches similar to this, as well as in various pretargeting formats. In one embodiment, for example, two or more species of targeting conjugates (fusion proteins) with antibodies directed to different epitopes and having non-overlapping cross-reactivity, each prepared according to the present invention, are administered according to the pretarget method disclosed herein, thereby improving diagnostic or therapeutic utility. A further embodiment utilizes the property that streptavidin monomers naturally associate to form tetramers. Thus, two or more antibodies, each conjugated (fused) to the monomeric form of streptavidin, are selectively combined and, upon formation of tetrameric streptavidin, yield single species with specificity for multiple epitopes at the target site.

It should be understood that the methods described herein may be modified and still achieve the desired effect. For example, two antibodies specific for the same antigen or cell type, regardless of their respective cross-reactivity, may be used. All that is necessary for these methods is that the targeting moiety-ligand/anti-ligand conjugate preferentially binds to the target cells and that the active agent substantially localizes to the pretargeted cells and is in certain embodiments otherwise substantially cleared from circulation.

Alternatively, antibody-based or non-antibody-based targeting moieties may be employed to deliver a ligand/anti-ligand to a target site bearing an unregulated antigen. Preferably, a natural binding agent for such an unregulated antigen is used for this purpose. Pretarget methods as described herein optionally include the administration of a clearing agent. The dosage of the clearing agent is an amount, which is sufficient to substantially clear the previously administered targeting moiety-ligand/anti-ligand conjugate from the circulation. Generally, the determination of when to administer the clearing agent depends on the target uptake and endogenous clearance of the targeting moiety conjugate. Particularly preferred clearing agents are those which provide for Ashwell receptor-mediated clearance, such as galactosylated proteins, e.g., galactosylated biotinylated human serum albumin, and small molecule clearing agents containing N-acetylgalactosamine and biotin.

Types of active agents (diagnostic or therapeutic) useful herein include radionuclides, toxins, anti-tumor agents, drugs, genes, and cytokines. For example, as described above, conjugates of such agents to biotin may be useful in the pretargeting approach. In this regard, a therapeutic antibody (e.g., an antibody that induces apoptosis or inhibits angiogenesis) may be used in a therapeutic modality such as pretargeting. With regard to diagnostic agent fusions, in contrast to therapeutic agent fusions, enhanced target cell internalization is disadvantageous if one administers diagnostic agent-targeting moiety conjugates. Internalization of diagnostic conjugates results in cellular catabolism and degradation of the conjugate. Upon degradation, small adducts of the diagnostic agent or the diagnostic agent per se may be released from the cell, thus eliminating the ability to detect the conjugate in a target-specific manner.

Diagnostic or therapeutic agents useful herein include radionuclides, drugs, anti-tumor agents, toxins, genes, and cytokines. Radionuclides useful within the present invention include gamma-emitters, positron-emitters, Auger electron-emitters, X-ray emitters and fluorescence-emitters, with beta- or alpha-emitters preferred for therapeutic use. Radionuclides are well-known in the art and include ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸Y, ⁹⁷Ru, ¹⁰⁰Pd, ^{101m}Rh, ¹¹⁹Sb, ¹²⁸Ba, ¹⁹⁷Hg, ²¹¹At, ²¹²Bi, ¹⁵³Sm, ¹⁶⁹Eu, ²¹²Pb, ¹⁰⁹Pd, ¹¹¹In, ⁶⁷Ga, ⁶⁸Ga, ⁶⁴Cu, ⁶⁷Cu, ⁷⁵Br, ⁷⁶Br, ⁷⁷Br, ^{99m}Tc, ¹¹C, ¹³N, ¹⁵O, ¹⁴⁹Pm, ¹⁶⁶Ho and ¹⁸F. Preferred therapeutic radionuclides include ¹⁸⁸Re, ¹⁸⁶Re, ²⁰³Pb, ²¹²Bi, ²¹³Bi, ¹⁰⁹Pd, ⁶⁴Cu, ⁶⁷Cu, 90Y, ¹²⁵I, ¹³¹I, ⁷⁷Br, ²¹¹At, ⁹⁷Ru, ¹⁰⁵Rh, ¹⁹⁸Au and ¹⁹⁹Ag, ¹⁴⁹Pm, ¹⁶⁶Ho or ¹⁷⁷Lu.

As one of ordinary skill in the art can readily appreciate the above streptavidin gene fusions may be utilized in combination therapies, such as when “pretargeting” is combined with the use of radiation-sensitizing agents. Such radiation sensitizing agents include, but are not limited to, Gemcitabine, 5-fluorouracil, paclitaxel, and the like.

Several of the potent toxins useful within the present invention consist of an A and a B chain. The A chain is the cytotoxic portion and the B chain is the receptor-binding portion of the intact toxin molecule (holotoxin). Because toxin B chain may mediate non-target cell binding, it is often advantageous to conjugate only the toxin A chain to a targeting moiety (e.g., molecule). However, while elimination of the toxin B chain decreases non-specific cytotoxicity, it also generally leads to decreased potency of the conjugated toxin A chain, as compared to the conjugate of the corresponding holotoxin.

Preferred toxins in this regard include holotoxins, such as abrin, ricin, modeccin, Pseudomonas exotoxin A, Diphtheria toxin, pertussis toxin, Shiga toxin, and bryototoxin; and A chain or “A chain-like” molecules, such as ricin A chain, abrin A chain, modeccin A chain, the enzymatic portion of Pseudomonas exotoxin A, Diphtheria toxin A chain, the enzymatic portion of pertussis toxin, the enzymatic portion of Shiga toxin, gelonin, pokeweed antiviral protein, saporin, tritin, barley toxin and snake venom peptides. Ribosomal inactivating proteins (RIPs), naturally occurring protein synthesis inhibitors that lack translocating and cell-binding ability, are also suitable for use herein. Highly toxic toxins, such as palytoxin and the like, are also contemplated for use in the practice of the present invention. However, therapeutic drugs may themselves facilitate internalization of the complex.

Therapeutic drugs, administered as targeted conjugates, are also encompassed herein. Again, the goal is administration of the highest possible concentration of drug (to maximize exposure of target tissue), while remaining below the threshold of unacceptable normal organ toxicity (due to non-target tissue exposure). Unlike radioisotopes, however, many therapeutic drugs need to be taken into a target cell to exert a cytotoxic effect. In the case of targeting moiety-therapeutic drug conjugates, it would be advantageous to combine the relative target specificity of a targeting moiety with a means for enhanced target cell internalization of the targeting moiety-drug conjugate.

Therapeutic drugs suitable for use herein include conventional chemo-therapeutics, such as vinblastine, doxorubicin, bleomycin, methotrexate, 5-fluorouracil, 6-thioguanine, cytarabine, cyclophosphamide and cis-platinum, as well as other conventional chemotherapeutics including those described in Cancer: Principles and Practice of Oncology, 2d ed., V. T. DeVita, Jr., S. Hellman, S. A. Rosenberg, J. B. Lippincott Co., Philadelphia, Pa., 1985, Chapter 14, and analogues of such drugs where the analogue has greater potency that the parent molecule. Another drug within the present invention is a trichothecene. Other preferred drugs suitable for use herein as a diagnostic or therapeutic active agent in the practice of the present invention include experimental drugs including those as described in NCI Investigational Drugs, Pharmaceutical Data 1987, NIH Publication No. 88-2141, Revised November 1987.

Other anti-tumor agents, e.g., agents active against proliferating cells, are administerable in accordance with the present invention. Exemplary anti-tumor agents include pro-apoptotic antibodies, anti-angiogenic antibodies, cytokines, such as IL-2, tumor necrosis factor or the like, lectin inflammatory response promoters (selecting), such as L-selectin, E-selectin, P-selectin or the like, and similar molecules.

One skilled in the art, based on the teachings in this application and the applications referenced herein, can readily determine an effective diagnostic or therapeutic dosage and treatment protocol. This will depend upon factors such as the particular selected therapeutic or diagnostic agent, the route of delivery, the type of target site(s), affinity of the targeting moiety for the target site of interest, any cross-reactivity of the targeting moiety with normal tissue, condition of the patient, whether the treatment is effected alone or in combination with other treatments, among other factors. A therapeutic effective dosage is one that treats a patient by extending the survival time of the patient. Preferably, the therapy further treats the patient by arresting the tumor growth and, most preferably, the therapy further eradicates the tumor.

All the references, including patents and patent applications, discussed throughout, are hereby incorporated by reference in their entirety.

The present invention is further described through presentation of the following examples. These examples are offered by way of illustration and not by way of limitation.

### EXAMPLES

### Example I

### Construction of huNR-LU-10 Single Chain Antibody-Genomic Streptavidin Fusion

Generically, a single chain Fv/streptavidin (scFvSA) fusion protein is expressed from the genetic fusion of the single chain antibody of the variable regions (scFv) to the genomic streptavidin of Streptomyces avidinii. The scFv gene consists of the variable regions of the light (V_{L}) and heavy (V_{H}) chains separated by a DNA linker sequence (e.g., FIG. 2). The streptavidin coding sequence is joined to the 3′ terminus of the scFv gene, and the two genes are separated in-frame by a second DNA linker sequence. The signal sequence from the streptavidin gene is fused at the 5′ terminus of the scFvSA gene to direct expression to the E. coli periplasmic space. The scFvSA gene is under control of the lac promoter, and the expressed fusion protein is extracted and purified from E. coli and forms a soluble tetramer of about 173,000 molecular weight.

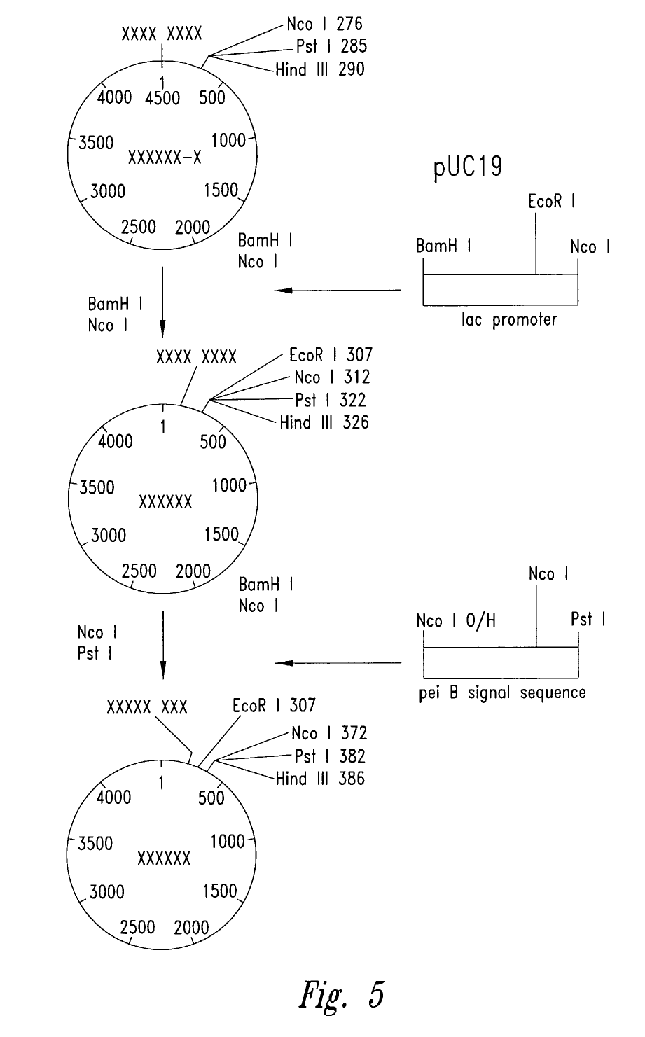

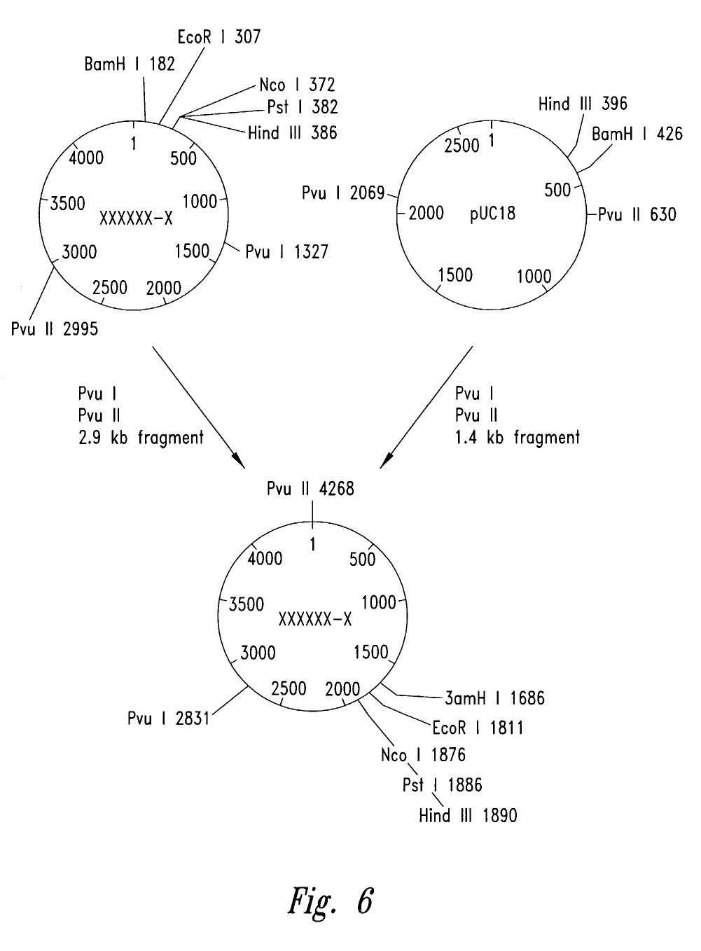

Plasmid pKK233-2 (Amersham Pharmacia Biotech, Piscataway, N.J.) was digested with BamHI and NcoI to remove the trc promoter. The lac promoter was amplified from pBR322 by polymerase chain reaction (PCR) and cloned into the BamHI/NcoI site of pKK233-2. In the process an EcoRI site was introduced immediately 5′ to the NcoI site. The plasmid was digested with NcoI and PstI and ligated with oligonucleotides encoding the pelB leader sequence. The accepting NcoI site on the plasmid was not regenerated and a new NcoI site was introduced in the 3′ area of the pelB encoding sequence. The resulting plasmid was referred to as pKK-lac/pelB (FIG. 5). pKK-lac/pelB and pUC18 were digested with PvuI and PvuII. The 2.9 kb fragment of pKK-lac/pelB containing the lac promoter and multi-cloning site was ligated to the 1.4 kb fragment of pUC18 containing the origin of replication to form plasmid pEX-1 (FIG. 6).

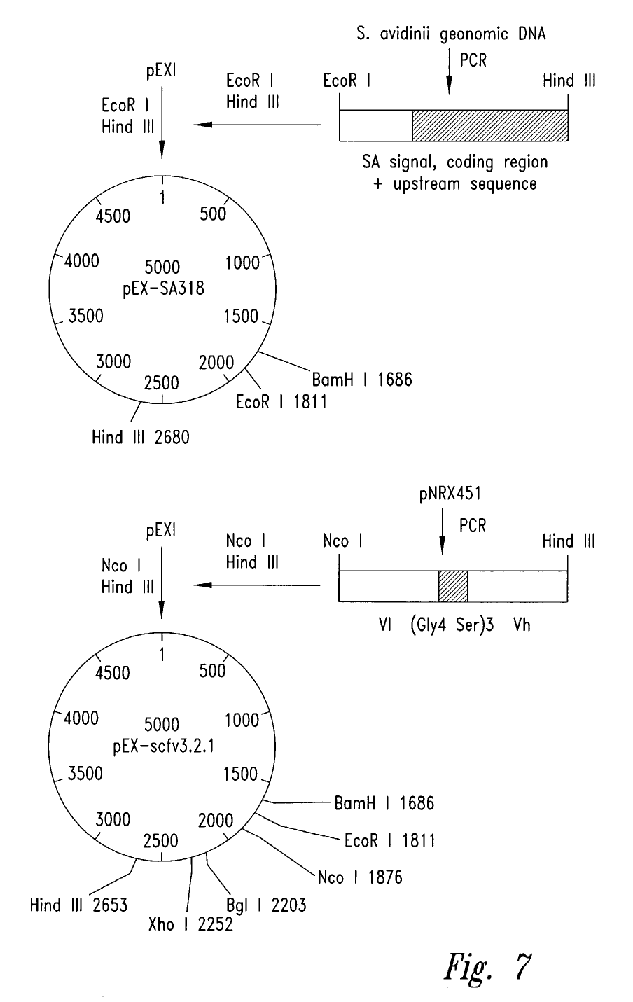

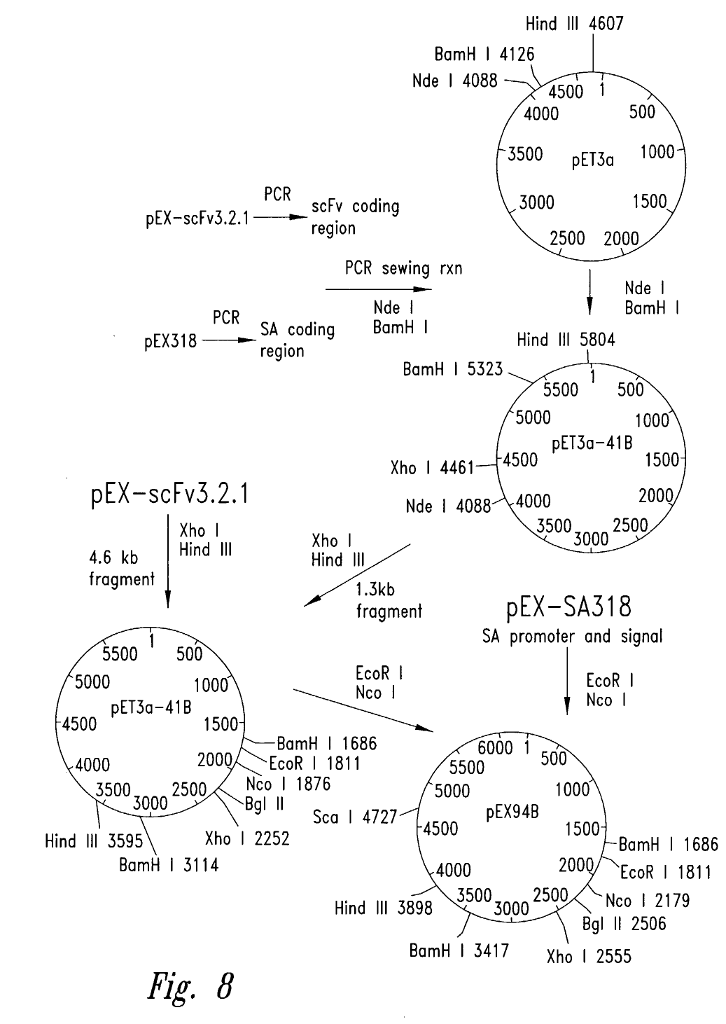

The streptavidin and huNR-LU-10 scFv genes (a monoclonal antibody that binds the antigen EGP40 or EPCAM, epithelial glycoprotein, 40 kD) were cloned onto separate plasmids prior to construction of the huNR-LU-10 scFvSA gene. The streptavidin gene, signal sequence and approximately 300 bp of upstream sequence were PCR-amplified from Streptomyces avidinii (ATCC 27419) genomic DNA and cloned into pEX-1 as an EcoRI/HindIII fragment to form pEX318 (FIG. 7). The huNR-LU-10 scFv was derived from the humanized antibody plasmid pNRX451 (Graves et al., Clin. Cancer Res., 5:899-908, 1999). The heavy and light chain variable regions were PCR-amplified separately from pNRX451 and then combined in a subsequent PCR. Oligonucleotides used in this process were designed to introduce a (Gly₄Ser)₃ linker between the leading V_{L} and the trailing V_{H}. The resulting PCR product was cloned into pEX-1 as a NcoI/HindIII fragment forming the plasmid pEX-scFv3.2.1 (FIG. 7). The scFv and streptavidin genes were PCR-amplified from pEX-scFv3.2.1 and pEX318, respectively, and combined into a fusion, as illustrated in FIG. 8. The oligonucleotides used in these reactions created an overlap between the 3′ end of the leading scFv and the 5′ end of the trailing streptavidin, which encoded a five amino acid linker (GSGSA). The fragments were joined by PCR using the outside primers. The resulting 1.25 kb fragment was cloned into the NdeI and BamHI sites of vector pET3a (Novagen), generating pET3a-41B. This plasmid was digested with XhoI and HindIII, and the 1.3 kb fragment containing the V_{H}-SA coding region and transcription terminator was ligated to a 4.6 kb XhoI/HindIII fragment of pEX-scFv3.2.1 containing the V_{L} coding region, lac promoter, and ampicillin resistance gene (pYL256). The streptavidin regulatory region and signal sequence were PCR-amplified from pEX318 and cloned into the EcoRI/NcoI sites of pYL256 to form pEX94B (FIG. 8).

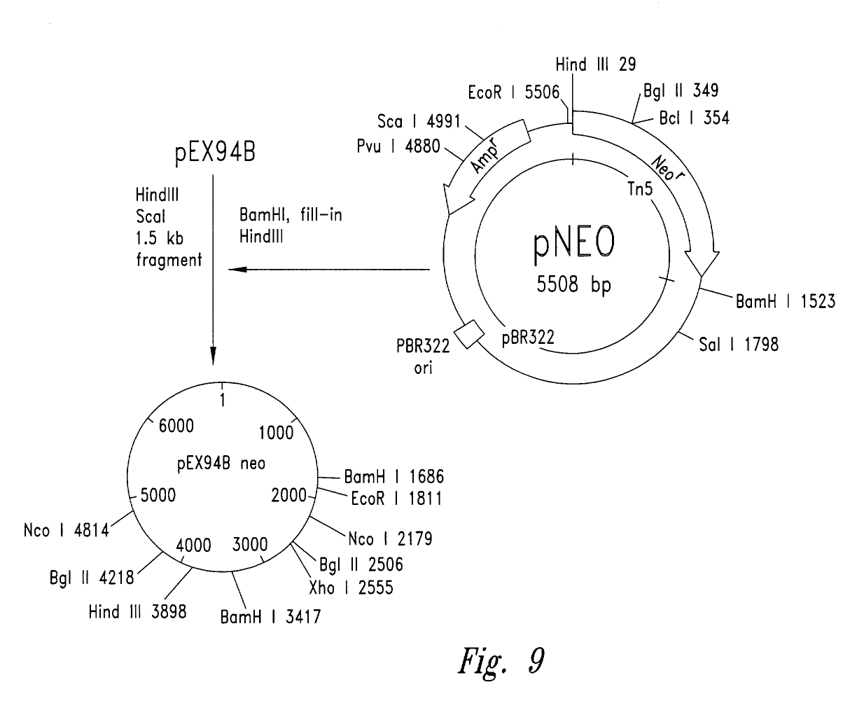

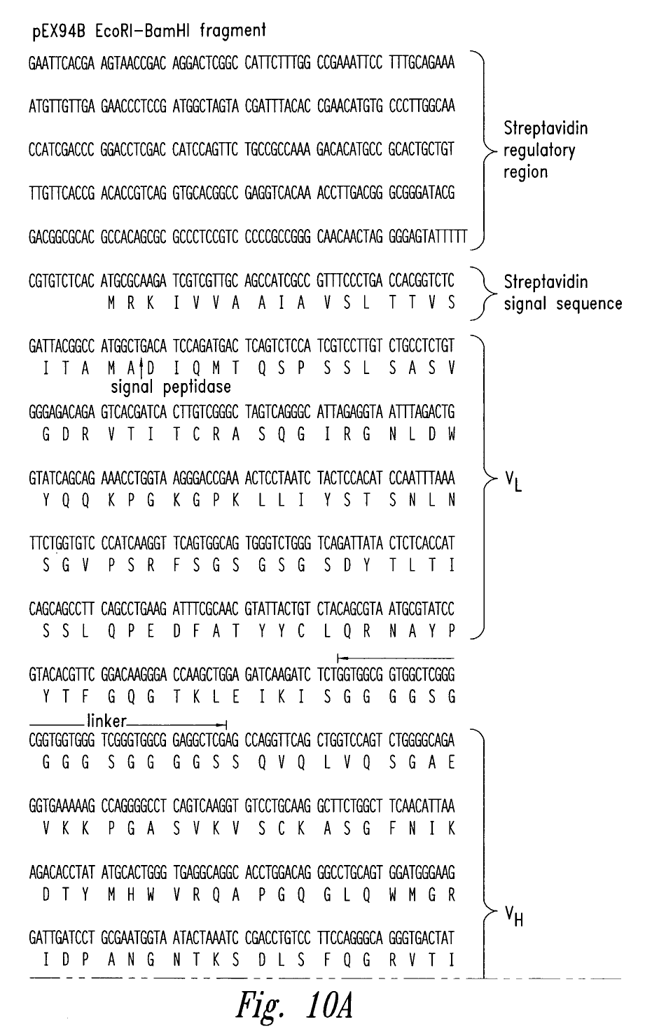

The Tn5 kanamycin resistance gene (neo) was inserted into the huNR-LU-10 scFvSA expression plasmid pEX94B as follows (FIG. 9): plasmid pNEO (Amersham Pharmacia) was digested with BamHI, blunt-ended with nucleotides using Pfu polymerase (Stratagene, La Jolla, Calif.), then further digested with HindIII. The 1494 bp fragment containing the kanamycin resistance gene was ligated to HindIII/ScaI-digested pEX94B plasmid, generating plasmid pEX94Bneo. The DNA sequence of the 1.6 kb EcoRI to BamHI fragment of plasmids pEX94B and pEX94Bneo is shown in FIG. 10.

### Example II

### Construction of B9E9 scFvSA Fusions

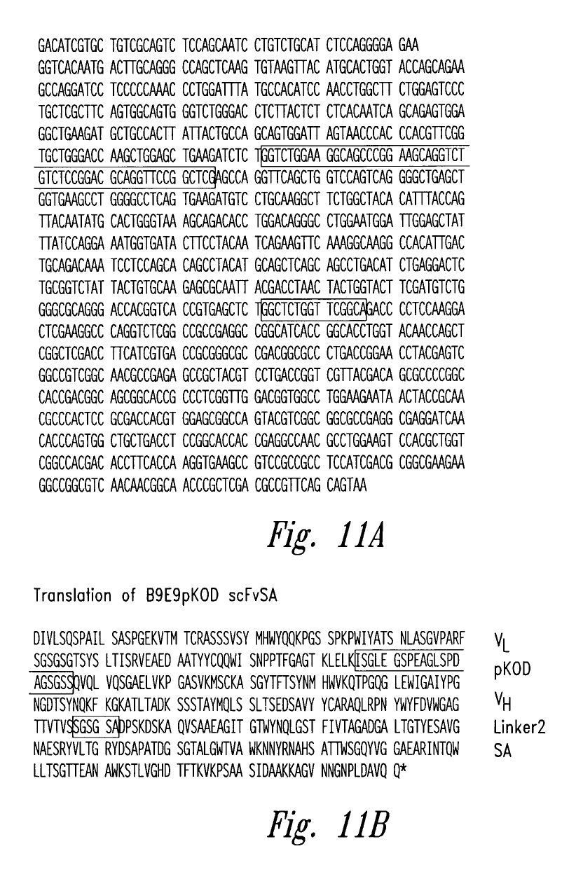

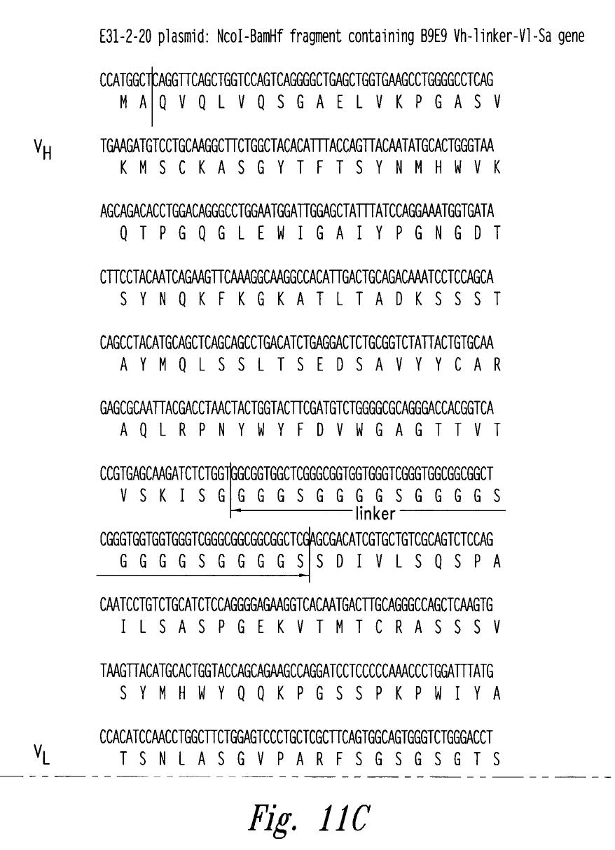

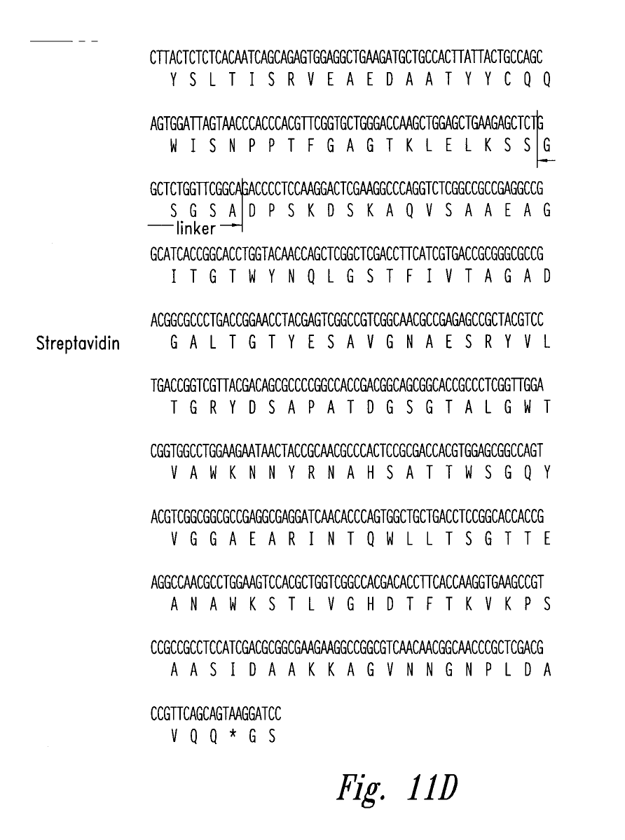

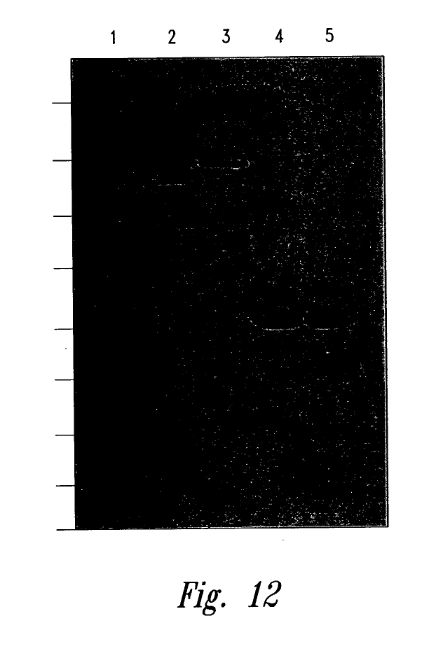

Additional single chain antibodies containing genomic streptavidin were constructed in a similar manner as noted above. A scFvSA version of the anti-CD20 mAb, B9E9, was constructed in the V_{L}V_{H} orientation with either a (Gly₄Ser)₃ (SEQ ID NO: 10)linker or a linker termed pKOD (amino acids GLEGSPEAGLSPDAGSGS) (SEQ ID NO: 9). Briefly, B9E9-1D3 hybridoma cells (1×10⁷)(from Bioprobe BV, Amstelveen, The Netherlands) were harvested, and total RNA was prepared. The cDNAs for kappa chain and heavy chain of B9E9 were obtained by a reverse transcriptase reaction using primers RX207 and RX215, respectively. PCR fragments of variable regions of kappa chain and heavy chain were obtained using above cDNAs and pairs of oligos (RX207 and NX54 for kappa chain; RX215 and NX50 for heavy chain). The PCR fragments were digested with EcoRI and NotI and subsequently cloned into a pPICUA vector (Invitrogen, Sorrento Valley, Calif.), previously restricted with EcoRI and NotI. The resultant plasmids C58-1 and C58-16 carried B9E9 kappa chain and heavy chain, respectively. The two chains were further cloned out from C58-1 and C58-16 by PCR using pairs of oligos (RX468 and RX469 for kappa chain; RX470 and RX471 for heavy chain). The kappa chain fragment was digested with NcoI and BgIII and the heavy chain was digested with XhoI-SacI, respectively. The kappa chain was cloned into pEX94B (NcoI-BgIII) as vector and heavy chain was cloned at XhoI-SacI sites in pEX94B. The resultant plasmids (C74-2 for kappa chain and C76-10 for heavy chain) were digested with XhoI and HindIII. The small fragment from C76-10 was ligated into C74-2 vector restricted with the same enzymes. A resultant plasmid (C87-14) carried B9E9 scFvSA fusion protein with a (G₄S)₃ (SEQ ID NO: 10) linker between kappa chain and heavy chain. The C87-14 was further digested with BgIII and XhoI and ligated with a pKOD linker prepared with two oligos (pInew5′ and pInew3′) to generate C136-1. FIGS. 11A and 11B illustrate the determined nucleic acid sequence and predicted amino acid sequence of B9E9pKOD scFvSA.

Another version of B9E9 scFvSA was constructed in the V_{H}V_{L} orientation with an extended 25mer (Gly₄Ser), (SEQ ID NO: 11) linker. The NcoI-SacI fragment of C87-14 containing scFv was further subcloned by PCR using a pair of primers (RX633 and RX471) to add a serine residue in the V_{L} region. The PCR fragment was digested with NcoI and SacI and cloned into the pEX94B vector restricted with NcoI and SacI. The resultant plasmid D59-3 was subject to subcloning to generate the V_{H} or V_{L} fragments by PCR using RX781 and RX782 or RX729 and RX780, respectively. The V_{H} PCR fragment was digested with NcoI and BgIII and cloned into the pEX94B vector at the same sites to form D142-6. The V_{L} PCR fragment was digested with XhoI and SacI and cloned into the pEX94B vector at the same sites to form D142-1. A XhoI-HindIII fragment from D142-1 was isolated and replaced a XhoI-HindIII fragment of D142-6 to generate D148-1 (V_{H}-V_{L} scFvSA). A HindIII-BamHI fragment, (blunted at BamHI side) containing a neo gene as described previously, was used to replace a HindIII-ScaI fragment of D148-1 to form D164-13. The D148-1 was also digested with BgIII and XhoI to remove the linker fragment and ligated with a 25mer linker (annealed with RX838 and RX839) to form E5-2-6. A EcoRI-HindIII fragment of E5-2-6 containing V_{H}-V_{L} scFvSA was excised and ligated with the D164-13 vector previously restricted with EcoRI and HindIII to form E31-2-20. Both plasmids E5-2-6 (carbenicillin-resistant) and E31-2-20 (kanamycin-resistant) express the B9E9 scFvSA fusion protein. FIG. 11C illustrates the nucleic acid sequence and predicted amino acid sequence of B9E9 scFvSA (V_{H}-V_{L} 25-mer).

All oligonucleotide primers, as listed below, were synthesized by Operon Technologies, Inc. (Alameda, Calif.).

| | |
|---|---|
| NX50 | |
| TGCCGTGAATTCGTSMARCTGCAGSARTCWGG | (SEQ ID NO: 12) |
| NX54 | |
| TGCCGTGAATTCCATTSWGCTGACCARTCTC | (SEQ ID NO: 13) |
| RX207 | |
| TAGCTGGCGGCCGCCCTGTTGAAGCTCTTGACAAT | (SEQ ID NO: 14) |
| RX215 | |
| TAGCTGGCGGCCGCTTTCTTGTCCACCTTGGTGC | (SEQ ID NO: 15) |
| RX468 | |
| TTACGGCCATGGCTGACATCGTGCTGCAGTCTCCAGCAATCCTGTCT | (SEQ ID NO: 16) |
| RX469 | |
| CACCAGAGATCTTCAGCTCCAGCTTGGTCCCA | (SEQ ID NO: 17) |
| RX470 | |
| CGGAGGCTCGAGCCAGGTTCAGCTGGTCCAGTCAGGGGCTGAGCTGGTGAAG | (SEQ ID NO: 18) |
| RX471 | |
| GAGCCAGAGCTCACGGTGACCGTGGTCCCTGCGCCCCA | (SEQ ID NO: 19) |
| pInew5′ | |
| GATCTCTGGTCTGGAAGGCAGCCCGGAAGCAGGTCTGTCTCCGGACGCAGGTTCCGGC | (SEQ ID NO: 20) |
| pInew3′ | |
| TCGAGCCGGAACCTGCGTCCGGAGACAGACCTGCTTCCGGGCTGCCTTCCAGACCAGA | (SEQ ID NO: 21) |
| RX633 | |
| TTACGGCCATGGCTGACATCGTGCTGTCGCAGTCTCCAGCAATCCTGTCT | (SEQ ID NO:22) |
| RX779 | |
| TTCCGGCTCGAGCGACATCGTGCTGTCGCAGTCTCCA | (SEQ ID NO: 23) |
| RX780 | |
| GAGCCAGAGCTCTTCAGCTCCAGCTTGGTCCC | (SEQ ID NO: 24) |
| RX781 | |
| TTACGGCCATGGCTCAGGTTCAGCTGGTCCAGTCA | (SEQ ID NO: 25) |
| RX782 | |
| AGACCAGAGATCTTGCTCACGGTGACCGTGGTCCC | (SEQ ID NO: 26) |
| RX838 | |
| GATCTCTGGTGGCGGTGGCTCGGGCGGTGGTGGGTCGGGTGGCGGCGGCTCGGGTGGTGGT | (SEQ ID NO: 27) |
| GGGTCGGGCGGCGGCGGC | |
| RX839 | |
| TCGAGCCGCCGCCGCCCGACCCACCACCACCCGAGCCGCCGCCACCCGACCCACCACCGCC | (SEQ ID NO: 28) |
| CGAGCCACCGCCACCAGA | |

### Example III

### Expression of huNR-LU-10 scFvSA and B9E9 scFvSA Proteins

Transformants of E. coli strain XL1-Blue (Stratagene, La Jolla, Calif.) containing plasmids pEX94B (huNR-LU-10 scFvSA) or E5-2-6 (B9E9 scFvSA) were grown overnight at 30° C. in Terrific broth (20 ml; Sigma) containing carbenicillin (50 μg/ml). The culture was diluted 100-fold into fresh medium and grown in a shaking incubator at 30° C. When the culture attained an A₆₀₀ of 0.3-0.5, IPTG (Amersham Pharmacia Biotech, Piscataway, N.J.) was added to a final concentration of 0.2 mM, and incubation was continued overnight. Periplasmic extracts were prepared for qualitative analysis of the scFvSA expression level. Cells were resuspended in an ice-cold solution of 20% sucrose, 2 mM EDTA, 30 mM Tris, (pH 8.0), and lysozyme (2.9 mg/ml) and were incubated on ice for 30 min. Supernatants were analyzed on 4-20% Tris-glycine SDS-PAGE gels (Novex) under non-reducing, non-boiled conditions, and gels were stained with Coomassie Blue. Expression using shake flask cultures was optimized by testing different environmental parameters, such as IPTG concentration and timing, temperature, media, or carbon source, or testing genetic factors, such as different promoters or signal sequences.

Clones were further grown in an 8L fermentor and analyzed for expression level. The primary inoculum (50 ml) was grown overnight at 30° C. in shake flasks containing Terrific broth plus 50 μg/ml kanamycin (plasmids pEX94Bneo or E31-2-20) or carbenicillin (plasmids pEX94B or E5-2-6), depending on the selectable marker of the plasmid. The culture was then diluted 100-fold into the same medium and grown at 30° C. for an additional 4-5 h. This secondary inoculum (0.5 liter) was transferred to a 14 liter BioFlo 3000 fermentor (New Brunswick Scientific) containing 8 liters of complete E. coli medium [per liter: 6 g Na₂HPO₄, 3 g KH₂PO₄, 0.5 g NaCl, 3 g (NH₄)₂SO₄, 48 g yeast extract (Difco), 0.25 ml Mazu DF204 antifoam (PPG Industries Inc., Pittsburgh, Pa.), 0.79 g MgSO₄-7H₂O, 0.044 g CaCl₂-2H₂O, and 3 ml of trace elements (per liter: 0.23 g CoCl₂, 0.57 g H₃BO₃, 0.2 g CuCl₂-2H₂O, 3.5 g FeCl₃-6H₂O, 4.0 g MnCl₂-4H₂O, 0.5 g ZnCl, 1.35 g thiamine, and 0.5 g Na₂MoO₄-2H₂O)]. The medium contained an initial 5 g/liter galactose as carbon source plus 50 μg/ml of kanamycin or carbenicillin for plasmid retention. The culture was grown at 30° C. and induced with IPTG (0.2 mM) at 6 h post-inoculation. The pH was maintained at 7.0 by the automatic addition of either phosphoric acid or NaOH. Dissolved oxygen concentration was maintained at or above 30% throughout the run using agitation speeds of 400-800 rpm and oxygen supplementation as necessary. A galactose solution (50%) was fed over a 9 h period after exhaustion of the initial galactose present in the medium to a total of 20-25 g per liter. Cells were harvested at 24-26 h post-inoculation (for B9E9 scFvSA) or 48-56 h post-inoculation (for huNR-LU-10 scFvSA) in a continuous flow centrifuge (Pilot Powerfuge, Carr Separations, Franklin, Mass.), washed with PBS (10 mM sodium phosphate, 150 mM NaCl, pH 7.2), and pelleted by centrifugation. A typical fermentation produced 80-90 g of cells (wet wt) per liter culture medium.

For determining expression levels, cells were washed twice in PBS, resuspended to the original volume, and disrupted either by sonication on ice (Branson Ultrasonics, Danbury, Conn.) or through two cycles of microfluidization (Microfluidics International, Newton, Mass.). Two assays were used for quantitating fusion protein in the supernatent of a centrifuged sample of crude lysate. Initially, an ELISA assay was used in which biotinylated albumin (100 ng per well in PBS) was coated overnight in 96-well plates at 4° C. and incubated with serial two-fold dilutions of either HPLC-purified fusion protein (200 ng/ml) or test samples. Detection was accomplished using peroxidase-labeled goat anti-streptavidin polyclonal antibody (Zymed, So. San Francisco, Calif.) with substrate buffer ABTS (Sigma). Plates were read at 415/490 nm with a dual wavelength automated plate reader. A first order, log x/log y regression analysis was performed for quantitation of the fusion protein.

Alternatively, a rhodamine-biotin HPLC assay was devised that provided faster results. The fusion protein in centrifuged lysates was complexed with excess rhodamine-derivatized biotin, which was prepared as follows: 5-(and-6-)-carboxytetramethylrhodamine, succinimidyl ester (Molecular Probes, Eugene Oreg.) was coupled to biocytin (Pierce, Rockford Ill.) through the formation of a stable amide bond. The reaction mixture was purified by HPLC using a Dynamax semi-preparative C-18 column (Rainin Instrument Co., Woburn, Mass.). The effluent was monitored at 547 nm and peak fractions collected and analyzed by mass spectrometry. Fractions corresponding in molecular weight to biocytin-rhodamine conjugate were pooled and concentrated by roto-evaporation (Buchii, Switzerland). An excess of purified biocytin-rhodamine conjugate was added to the clarified crude lysate and analyzed by size exclusion chromatography using a Zorbax GF-250 column (MAC-MOD, Chadds Ford, Pa.) equilibrated in 20 mM sodium phosphate containing 15% DMSO at 1.0 ml/min flow rate. The effluent was monitored at 547 nm using a Varian Dynamax PDA-2 detector, and the peak area corresponding to fusion protein elution was determined using a Varian Dynamax HPLC Data System (Walnut Creek, Calif.). The concentration of fusion protein in the crude lysate was calculated by comparison to a standard analyzed under the same conditions. The molar extinction coefficient for the fusion protein standard was calculated using a previously described method summing the relative contributions of amino acids absorbing at 280 nm (Gill and von Hippel, Analyt. Chem. 182:319-326, 1989).

Expression levels in fermentor-grown cells were 100-130 mg/liter for huNR-LU-10 scFvSA, 40 mg/liter for B9E9pKOD scFvSA, and 270-300 mg/liter for B9E9 scFvSA (V_{H}-V_{L} 25-mer).

### Example IV

### Expression of B9E9 scFvSA Using Various Linkers and Signal Sequences

A number of genetic variants were constructed that contained linkers of different lengths and composition and the variable regions in different order (Table 1). These constructs were initially grown and induced in shake flask cultures and qualitatively assessed for expression by visualizing periplasmic proteins on Coomassie-stained, non-reducing, SDS gels. High-expressing constructs were further tested in an 8L fermentor using a galactose fed-batch protocol, and their expression levels were quantitatively determined by size exclusion HPLC using rhodamine-derivatized biotin. The construct that best fulfilled these criteria contained a 25-mer Gly₄Ser linker with the scFv in the V_{H}V_{L} orientation.

TABLE 1
Summary of expression levels of B9E9 scFvSA genetic variants.

| | |
|---|---|
| V_{L}-V_{H}-SA | V_{H}-V_{L}-SA |

| Linker type^{a} | Expression^{b} | Linker type | Expression | |
|---|---|---|---|---|
| 15 mer G4S | + | 15 mer G4S | 60 | mg/L |
| 18 mer G4S | ++ | 18 mer G4S | 195 | mg/L |
| 25 mer G4S | ++ | 25 mer G4S | 300 | mg/L |
| 35 mer G4S | ++ | | | |
| 18 mer pKOD | 40 mg/L | 18 mer pKOD | 60 | mg/L |
| 18 mer pKOD2 | ++ | 18 mer pKOD2 | +++ | |

Linker sequences (L1):
15 mer G4S: GGGGSGGGGSGGGGS (SEQ ID NO: 10)
18 mer G4S: GGGGSGGGGSGGGGSGGS (SEQ ID NO: 29)
25 mer G4S: GGGGSGGGGSGGGGSGGGGSGGGGS (SEQ ID NO: 11)
35 mer G4S: 7 copies of GGGGS (SEQ ID NO: 30)
18 mer pKOD: GLEGSPEAGLSPDAGSGS (SEQ ID NO: 9)
18 mer pKOD2: GLEGSPEAGLSPDAGSDS (SEQ ID NO: 31)
The expression levels of the fusion proteins were based on 8L fermentor runs except for those qualitative data, designated as plus symbols, based on SDS-PAGE analysis of shake-flask cultures.

### Example V

### Increased Expression of scFvSA Fusion Proteins in Periplasm of E. coli

The E. coli fkpA gene is a member of the family of FK506-binding proteins (FKBPs) and is one of the periplasmic components involved in protein folding. It is expressed in the E. coli periplasm and has peptidyl-prolyl isomerase (PPIase) activity. The PPIase-independent chaperone activity of the FkpA gene product has also been demonstrated both in vivo and in vitro. The FkpA chaperone protein is involved in a protein-folding process by stabilizing the folding intermediates in the periplasm. It was tested whether co-expression of the single chaperone gene (fkpA) was able to stimulate the expression of scFvSA fusion proteins, especially among those that had not previously expressed well in E. coli.

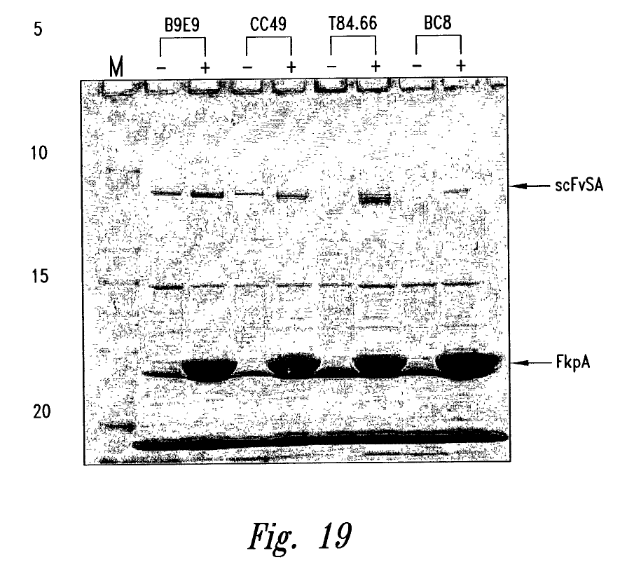

In order to clone the DNA fragment of the fkpA gene, chromosomal DNA was extracted from E. coli XL1-Blue cells (Stratagene) and digested with XhoI. Thirty-five cycles of PCR were performed using a pair of oligonucleotides (RX1229: ACGACGGTTGCTGCGGCGGTC (SEQ ID NO: 32); RX1231: AGGCTCATTAAT GATGCGGGT (SEQ ID NO: 33); both obtained from Operon Technologies, Inc.) and 300 ng of the digested genomic DNA as a template. The PCR mixture was subject to a second round of PCR (30 cycles) using a pair of nested oligonucleotides (RX1230: GGATCCAAGCTTACGATCACGGTCATGMCACG (SEQ ID NO: 34); RX1232: CTCGAGAAGCTTTAACTAAATTAATACAGCGGA) (SEQ ID NO: 35). The PCR fragments were resolved on a 1% agarose gel, and the 1.6-kb fragment was isolated. The extracted DNA was cloned into the TA vector (Invitrogen), and the sequence was confirmed by DNA sequencing. The clone was digested with HindIII, using a site that was incorporated into oligonucleotides RX1230 and RX 1232 and was ligated with HindIII-digested vector E84-2-8 (NeoRx Corp.), harboring the anti-CEA T84.66 scFvSA fusion gene (T84.66 cDNA from City of Hope, Duarte, Calif.). The resultant plasmid (F115-1-1) was used to transform XL1-Blue E. coli for shake-flask expression. The periplasmic components were extracted and analyzed on 4-20% SDS-PAGE. For electrophoretic analysis, 20 μl of the solution of scFvSA periplasmic fusion proteins were loaded in each lane of the gel. Following electrophoresis, the gel was stained with Coomassie Blue R250. The FkpA protein, with a molecular weight of about 30,000, was prominently present in all samples carrying the fkpA gene (+), while absent in those lacking the gene (−), as shown, for example, in FIG. 19. The molecular weights of the seven components in the SeeBlue molecular standard marker (M), obtained from Novex, listed in order of increasing size, from the bottom of the gel, are 16,000; 30,000; 36,000; 50,000; 64,000; 98,000; and 250,000. As seen in FIG. 19, expression of the T84.66 scFvSA fusion protein increased dramatically when co-expressed with the FkpA chaperone protein, in comparison to the parent construct (E84-2-8) lacking the fkpA gene. Additional scFvSA fusions were constructed by moving NcoI-SacI fragments to the F115-1-1 vector, which had previously been restricted with NcoI and SacI. The resultant plasmids were tested in E. coli XL1-Blue shake flask cultures. Upon electrophoretic analysis, several showed increased fusion protein expression, as demonstrated in FIG. 19 and Table 2. The results summarized here involve only the Vₕ-Vₗ-SA fusion configuration incorporating the (Gly₄Ser)₅ (SEQ ID NO: 11) linker. As summarized in Table 2, the expression levels of fusion proteins in the shake flask experiments were estimated qualitatively, with the highest level assigned a level of +++++.

TABLE 2
Qualitative expression of scFvSA fusion proteins in E. coli.

| | |
|---|---|
| SEQ ID | Expression level |

| Antigen | scFvSA | NO. | FkpA⁻ | FkpA⁺ |
|---|---|---|---|---|
| CEA | T84.66 | 36 | − | ++ |
| | Col-1 | 37 | − | + |
| | PR1A3 | 38 | − | − |
| | MFE-23 | 39 | ++++ | ++ |
| | Nrco-2 | 40 | +++ | + |
| Tag-72 | CC49 | 41 | ++ | ++++ |
| MUC-1 | BrE-3 | 42 | − | −+ |
| c-erbB2 | ICR12 | 43 | − | − |
| CD20 | B9E9 | 44 | +++ | +++++ |
| | C2B8 | 45 | − | − |
| CD45 | BC8 | 46 | + | +++ |

### Example VI

### Purification of huNR-LU-10 scFvSA and B9E9 scFvSA Proteins

The iminobiotin affinity matrix was prepared by reacting epoxide-activated Macro-prep matrix (BioRad, Hercules, Calif.) with 112 μm N-(3-amino-propyl)-1,3 propane diamine (Sigma) per g of matrix in 0.2 M carbonate buffer. The reaction was stopped after 8 h by filtering the slurry through a scintered glass funnel and rinsing the matrix with distilled water. Residual epoxides were inactivated by reacting the matrix with 0.1 M sulfuric acid for 4 h at 80° C., and the matrix was again rinsed. The amine-derivitized matrix was suspended in PBS, and the pH increased to 8.5 by the addition of 10% volume of 0.5 M sodium borate, pH 8.5. NHS-iminobiotin (Pierce) was dissolved in DMSO and added to the suspended matrix at a ratio of 2.6 mg/g of matrix. Following a 4 h reaction, the matrix was rinsed with distilled water followed by several alternating washes with pH 11 sodium carbonate buffer and pH 4 sodium acetate buffer and a final rinse with distilled water. The matrix was stored as a slurry in 20% ethanol.

Cells (650-750 g, wet wt) were washed twice in PBS, resuspended to 10-20% weight per volume with ice-cold 30 mM Tris, 1 mM EDTA, pH 8, and disrupted through two cycles of microfluidization. The lysate was adjusted to 50 mM glycine, 450 mM NaCl, pH 9.6, with a conductivity range of 46-48 mSe per cm, and then centrifuged at 12,000 rpm for 90 min. The supernatant was filtered (0.2 μm), then affinity purified over immobilized iminobiotin. The iminobiotin matrix was packed in a column and equilibrated in 50 mM glycine, 500 mM NaCl, pH 9.6 with a conductivity of 46-48 mSe per cm. Capacity using recombinant streptavidin (Roche Biochemical, Indianapolis, Ind.) was 2 mg per ml of bed volume under a flow of 2 ml/cm²/min. The 0.2 μm filtered cell homogenized supernatant was pumped at room temperature at 2 ml/cm² per min using 80 ml of bed volume per 100 g of cells. After washing with 20 bed volumes of column equilibrating buffer, the scFvSA fusion protein was eluted with 0.2 M sodium acetate, 0.1 M NaCl, pH 4.0, neutralized with Tris buffer, and then exhaustively dialyzed in refrigerated PBS.

To reduce protein aggregation, purified scFvSA was treated with 10% DMSO for 5-7 h at room temperature and dialyzed in PBS. The purified protein was concentrated using an Amicon YM30 membrane apparatus and filter-sterilized for aseptic storage at 4° C. At concentrations of 2-3 mg/ml, purified preparations typically contained ca. 5-8% aggregate.

Typical recoveries from iminobiotin chromatography were 50-60% with less than 5% appearing in the flow-through and wash. The residual remained as aggregate/entrapped material on the column. Addition of DMSO to the eluting buffer yielded <5% additional purified protein. Use of a variety of ionic and nonionic detergents did not improve recoveries. HPLC size exclusion analysis of the eluted fusion protein showed that up to 40% of the protein was in an aggregated form. Light scattering HPLC indicated aggregate sizes between 400,000 and 4 million. Treatment with 10% DMSO for several hours resulted in the slow de-aggregation of the fusion protein, yielding >92% tetrameric species that remained so when stored refrigerated in PBS at a concentration of <3 mg/mL.

### Example VII

### Biochemical Characterization of huNR-LU-10 scFvSA and B9E9 scFvSA Proteins

SDS-PAGE Analysis. Purified fusion proteins were analyzed on 4-20% Tris-glycine SDS-PAGE gels (Novex, San Diego, Calif.) under nonreducing conditions. Before electrophoresis, samples were mixed with SDS-loading buffer and incubated at either room temperature or 95° C. for 5 min. Gels were stained with Coomassie blue.

SDS-PAGE demonstrated that the fusion proteins were purified to >95% homogeneity after iminobiotin chromatography (FIG. 12, lanes 2 & 3; huNR-LU-10 data only). The major band migrated at the expected molecular weight of ˜173 kDa with minor isoforms evident. These isoforms were also detected with polyclonal anti-streptavidin antibody on Western gel analysis (data not shown). However, all bands resolved into a single species of ˜43 kDa when the protein was boiled prior to electrophoresis, consistent with a single protein entity dissociable into its homogeneous subunit (FIG. 12, lanes 4 & 5). The molecular weights of the seven components in the SeeBlue molecular standard marker (FIG. 12, lane 1), available from Novex, are described in Example V.

Size exclusion HPLC and Laser Light Scattering Analysis. Purified protein preparations were analyzed by size exclusion HPLC performed on a Zorbax GF-250 column with a 20 mM sodium phosphate/0.5 M NaCl mobile phase. The molecular weight of the fusion construct was measured using this Zorbax system connected in series with a Varian Star 9040 refractive index detector and a MiniDawn light scattering instrument (Wyatt Technologies, Santa Barbara, Calif.). A dn/dc value of 0.185 for a protein in an aqueous buffer solution was used in the molecular weight calculations.

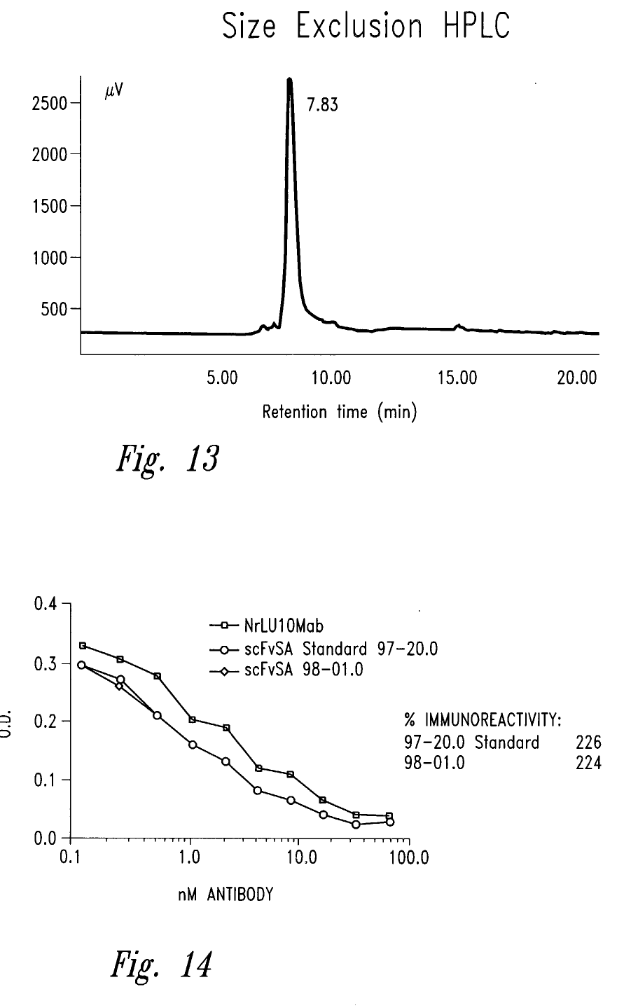

HPLC size exclusion chromatography exhibited a major peak with a retention time appropriate for the huNR-LU-10 tetramer with a minor (<8%) aggregate peak (FIG. 13). B9E9 scFvSA showed a very similar profile (graph not shown). These analyses demonstrated that all of the purified protein was tetrameric or an aggregate thereof. Light scattering analysis of huNR-LU-10 scFvSA indicated a molecular weight of 172,600, as predicted for the tetrameric protein.

Amino-terminal sequencing. Automated amino acid sequencing was performed using a Procise 494 sequenator (Applied Biosystems, Inc., Foster City, Calif.). This revealed that the leader sequences of both huNR-LU-10 scFvSA and B9E9 scFvSA were cleaved at the expected signal peptidase site adjacent to the first amino acid of the variable region.

Molecular weight determination of B9E9 scFvSA. Liquid chromatographic separation was conducted with an Hewlett Packard series 1100 system, fitted with a Jupiter C18 column (300 Δ, 3.2×50 mm, 5μ) and C18 “SafeGuard” column (Phenomenex, Torrance, Calif.) at a flow rate of 500 μl/min. The mobile phase was composed of water/1% formic acid (buffer A) and acetonitrile/1% formic acid (buffer B). The gradient applied was 2% B for 3 min rising to 99% B within 7 min. B9E9 scFvSA (10 μl) was eluted at a retention time of 8.7 min. The analytical column was interfaced with a Thermoquest/Finnigan ESI LCQ ion trap mass spectrometer (San Jose, Calif.). The instrument was calibrated with myoglobin and operated in the positive ion mode with the heated capillary set to 200° C. and 5.1 kV applied to the electrospray needle. The data were acquired in a full scan MS mode (m/z [500-2000 Da/z]) using automated gain control with 3 microscans and a maximum ion time of 500 ms.

The mass spectrum of the B9E9 monomer showed a deconvoluted molecular weight of 43,401, which is in agreement with the calculated most abundant mass of 43,400.

HuNR-LU-10 Competitive Immunoreactivity ELISA. Serial dilutions of the humanized NR-LU-10 whole antibody or the huNR-LU-10 fusion protein were allowed to compete with peroxidase-labeled murine NR-LU-10 whole antibody for binding to an 0.1% NP40 membrane extract from the human carcinoma cell line, LS-174 (ATCC #CL188). Following a log-logit transformation of the data in which curves were fit to the same slope, the concentration of competitor antibody that gave 50% inhibition (k) was calculated. Percent immunoreactivity was determined according the formula: k (fusion protein standard)/k (whole antibody standard)×100. The huNR-LU-10 fusion protein was found to possess immunoreactivity superior (˜225%) to the intact divalent humanized antibody (FIG. 14).

B9E9 Competitive Immunoreactivity FACS Assay. Immunoreactivity was assessed in a competitive binding assay using flow cytometry that measured the binding of fluorescein-labeled B9E9 to the CD20-positive Ramos cell line (Burkitt's lymphoma; ATCC CRL-1596) in the presence of various concentrations of unlabeled antibody. B9E9 mAb was labeled using fluorescein N-hydroxysuccinimidate, and an optimized amount of this conjugate was mixed with serial dilutions (3-200 ng/ml) of B9E9 mAb standard or molar equivalents of B9E9 scFvSA and incubated with 1×10⁶ cells at 4° C. for 30 minutes. Samples were washed and then analyzed on a single laser FACSCalibur (Becton Dickinson). After gating on single cells, the geometric mean fluorescence intensity was determined from a histogram plot of fluorescence. The concentration of competitor antibody required for 50% inhibition (IC₅₀) of fluorescein-B9E9 binding was calculated using nonlinear regression analysis for one-site binding.

Percent immunoreactivity=[IC₅₀scFvSA/IC₅₀ mAb]×100.

The scFvSA was about twice as immunoreactive (˜185%) as the divalent B9E9 antibody on a molar basis, and nearly equivalent (˜93%) to B9E9 mAb when adjusted for tetravalency (graph not shown).

B9E9 scFvSA Avidity. Avidity was determined using saturation binding experiments that measure specific binding of radiolabeled mAb or fusion protein (0.025-50 ng/ml) at equilibrium in the presence of excess antigen (10⁷ cells). Nonspecific binding was determined in the presence of excess cold mAb or fusion protein (50 μg/ml). Mixtures were incubated and centrifuged as described above. The equilibrium dissociation constant (Kd) was calculated from nonlinear regression analysis of nM bound vs. nM radioligand using immunoreactivity-adjusted antibody concentrations. The B9E9 fusion protein retained the same relative nanomolar avidity as the B9E9 mAb, as determined by radiolabeled binding to Ramos cells (Table 3).

TABLE 3
Avidity of B9E9 mAb and scFvSA fusion protein.

| Antibody | K_{d} (nM)^{a} | Kₐ (×10⁸ M⁻¹) |
|---|---|---|
| B9E9 mAb | 9.75 | 1.02 |
| B9E9 scFvSA (25-mer) | 12.44 | 0.80 |

^{a}Antibody concentrations were adjusted for immunoreactivity (67% and 79% for mAb and scFvSA, respectively). K_{d} and Kₐ were calculated using nonlinear regression analysis of nM bound vs. nM radioligand.

Biotin Binding and Dissociation. Biotin binding capacity was determined by incubation of a known quantity of fusion protein with a 9-fold molar excess of [³H]biotin (NEN Research Products, Boston, Mass.). After removal of uncomplexed biotin using streptavidin-immobilized beads (Pierce Chemical; Rockford, Ill.), the amount of [³H]biotin associated with the fusion protein was determined.

HuNR-LU-10 scFvSA and B9E9 scFvSA were capable of binding an average of 3.0 and 3.6 biotins, respectively, as compared to 4 biotin binding sites for recombinant streptavidin.

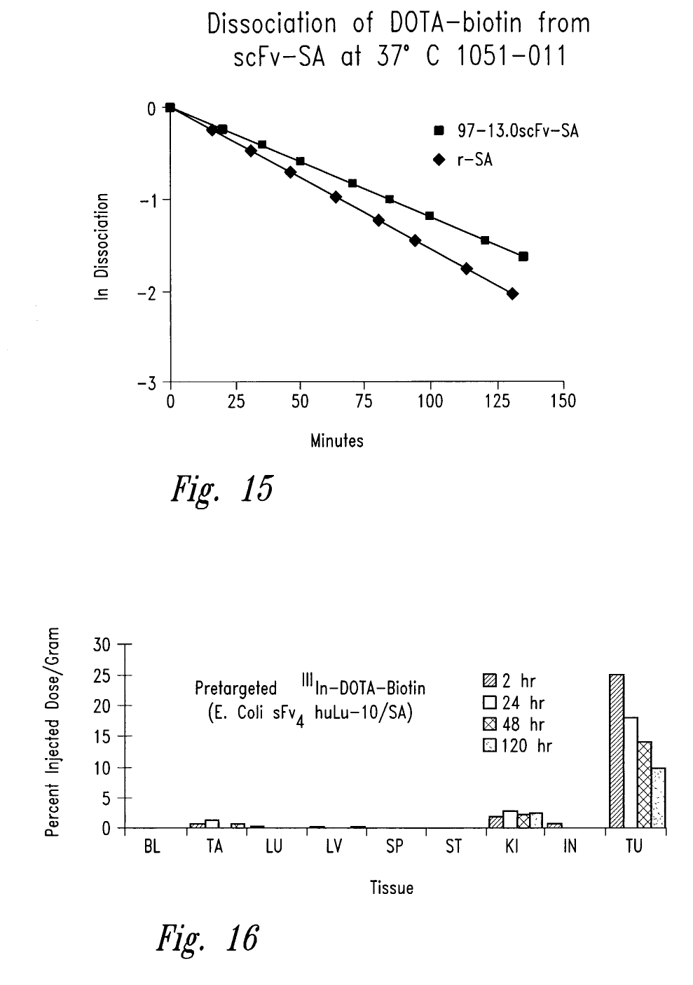

For huNR-LU-10 scFvSA, the rate of DOTA-biotin dissociation was assessed at 37° C. in 0.25 M phosphate, 0.15 M sodium chloride, pH 7.0 containing either 10 μM fusion protein or recombinant streptavidin and a subsaturating level of [⁹⁰Y]DOTA-biotin. A 100-fold saturating level of biocytin (Sigma) was added to initiate the dissociation measurement. At timed intervals, aliquots of incubate were diluted in PBS containing 0.5% bovine serum albumin. In order to precipitate the protein, zinc sulfate was added to each diluted aliquot, followed by sodium hydroxide, each to yield a final concentration of 0.06 M. Following microcentrifugation, free [⁹⁰Y]DOTA-biotin in the supernatant was assessed using a Hewlett Packard beta counter. The DOTA-biotin dissociation rate of huNR-LU-10 scFvSA was comparable to that of recombinant streptavidin (t_{1/2} of 58 min for huNR-LU-10 scFvSA vs. 47 min for recombinant streptavidin; FIG. 15).

For B9E9 scFvSA, biotin dissociation was measured as described above, except [³H]biotin was used instead of [⁹⁰Y]DOTA-biotin. The calculated t_{1/2} for biotin dissociation was 379 min for B9E9 scFvSA vs. 364 min for recombinant streptavidin (graph not shown).

### Example VIII

### Analysis of Biodistribution of ¹¹¹In-DOTA-Biotin After Pretargeting With huNR-LU-10 scFvSA

The expressed huNR-LU-10 scFvSA gene fusion was tested in a full pretarget protocol in female nude mice bearing SW-1222 human colon cancer xenografts (100-200 mg), subcutaneously implanted on the right flank. In these experiments, 575 μg of ¹²⁵I-labeled fusion protein was injected intravenous (iv) and allowed to circulate for 18 hours prior to iv injection of 100 μg of synthetic clearing agent (sCA) (See e.g., PCT Publication Nos. WO 97/46098 and WO 95/15978). Three hours after the sCA injection, there was an injection of 1.0 μg of ¹¹¹In-DOTA-biotin, essentially a chelating agent containing a radionuclide, conjugated to biotin (see U.S. Pat. Nos. 5,578,287 and 5,608,060). Mice were sampled for blood, then sacrificed and dissected at 2, 24, 48, and 120 hours after ¹¹¹In-DOTA-biotin injection.

The concentration of ¹²⁵I-huNR-LU-10 scFvSA radioactivity in blood and most well-perfused soft tissues was very low, due to the low blood pool concentration induced by the sCA complexation and subsequent hepatic clearance. The exceptions were liver and tumor. Liver uptake and retention of fusion protein was due to the mechanism of clearing agent action, and the somewhat retarded degradation of the streptavidin-containing fusion protein, which was consistent with similar results observed in studies of both streptavidin and the chemical conjugate of huNR-LU-10 and streptavidin (huNR-LU-10/SA) (data not shown). The ¹²⁵I-huNR-LU-10 scFvSA exhibited evidence of in vivo immunoreactivity by the retention of relatively high radiolabel concentration at the tumor (both stoichiometrically and relative to blood pool concentration) at all time points. The ratio of tumor concentration to blood concentration continuously increased from 23 to 143 hours. The lower blood pool values induced by clearing agent have led to a dramatic increase in the ratio, achieving average values over twice those observed in the absence of clearing agent (data not shown).

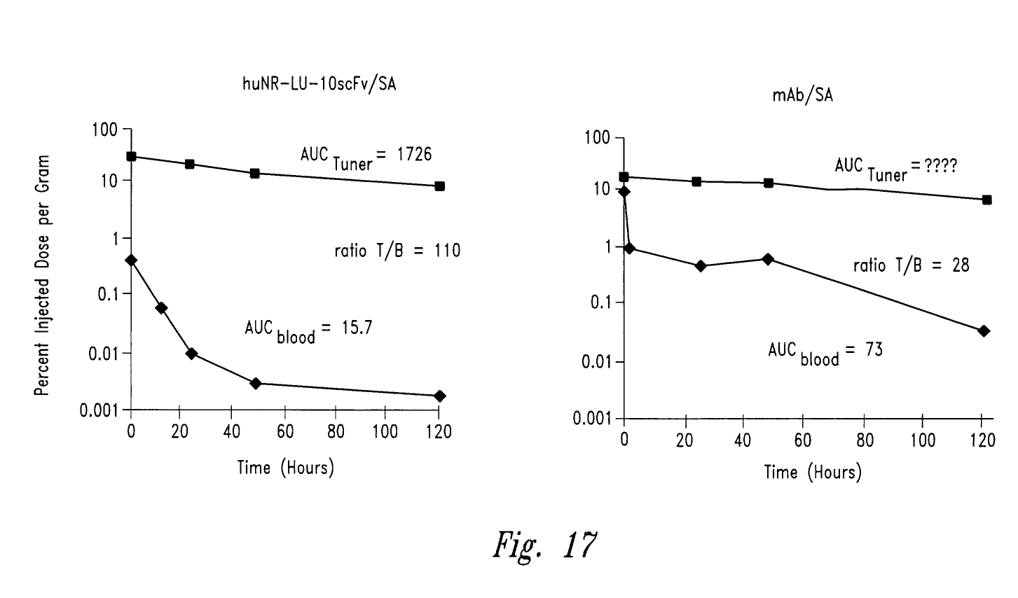

The pretargeted ¹¹¹In-DOTA-biotin biodistribution is shown in FIG. 16. Consistent with pretargeting results employing the chemical conjugate huNR-LU-10/SA, the concentration of ¹¹¹In-DOTA-biotin radioactivity in blood and all non-xenograft soft tissues was very low. Despite the high concentrations of fusion protein in the liver noted above, ¹¹¹In-DOTA-biotin uptake and retention in this organ was not evident, indicating that the fusion protein had been efficiently internalized and was unavailable to bind the subsequently administered radiobiotin. The highest concentration of ¹¹¹In-DOTA-biotin was at the tumor at all time points. (The tissues in order in FIG. 16 are blood, tail, lung, liver, spleen, stomach, kidney, intestine, and tumor.) The rapid uptake, achieving peak concentrations at the earliest time point sampled, is a hallmark of pretargeting. Efficient, consistent delivery and retention of ¹¹¹In-DOTA-biotin at the tumor was also observed. Peak concentrations of ¹¹¹In-DOTA-biotin at the tumor were within the range consistently achieved by use of the chemical NR-LU-10/SA conjugate (20-25% injected dose/g) (data not shown).

### Example IX

### Anaylsis of Blood Clearance and Tumor Uptake of huNR-LU-10 scFvSA Versus huNR-LU-10/Streptavidin Chemical Conjugate

Tumor to blood ratios of huNR-LU-10 scFvSA increased from nearly 100, two hours after DOTA-biotin injection, to several thousand by 24 hours. Comparative results for the huNR-LU-10/SA chemical conjugate and fusion protein, showing the efficiency of radiobiotin delivery to tumor and corresponding area-under-the-curve (AUC) values for blood, and tumor are shown in FIG. 17.

The overall tumor AUC using the fusion protein was somewhat less than that of the chemical conjugate (1726, for the time interval between 0-120 hours, versus 2047 for a typical chemical conjugate experiment). However, there was a dramatic difference in the concentration of ¹¹¹In-DOTA-biotin in the blood pool, with the concentration in the fusion protein group consistently lower at all time points. The greatest ramification of this decreased retention of radioactivity in the blood is that animals treated with the fusion protein experience a higher therapeutic index (tumor/blood) than those treated with the chemical conjugate.

### Example X

### Pretargeted Biodistribution of B9E9 scFvSA

Pretargeted radioimmunotherapy studies were conducted in female nude mice bearing well-established Ramos human cancer xenografts (100-400 mg). Tumored BkI:BALB/c/nu/nu nude mice were obtained by implanting 5-25×10⁶ cultured cells subcutaneously in the side midline 10-25 days prior to study initiation. Mice received intravenous injections of the ¹²⁵I-labeled B9E9 scFvSA (600 μg), and 20 hours later were injected intravenously with 100 μg of synthetic clearing agent. ¹¹¹In-labeled DOTA-biotin (1.0 μg) was injected intravenously into each mouse 4 hours after clearing agent. Groups of three mice per time point were bled and sacrificed at 2, 24, and 48 hours after injection of ¹¹¹In-DOTA-biotin. Whole organs and tissue were isolated, weighed, and counted for radioactivity using a gamma counter.

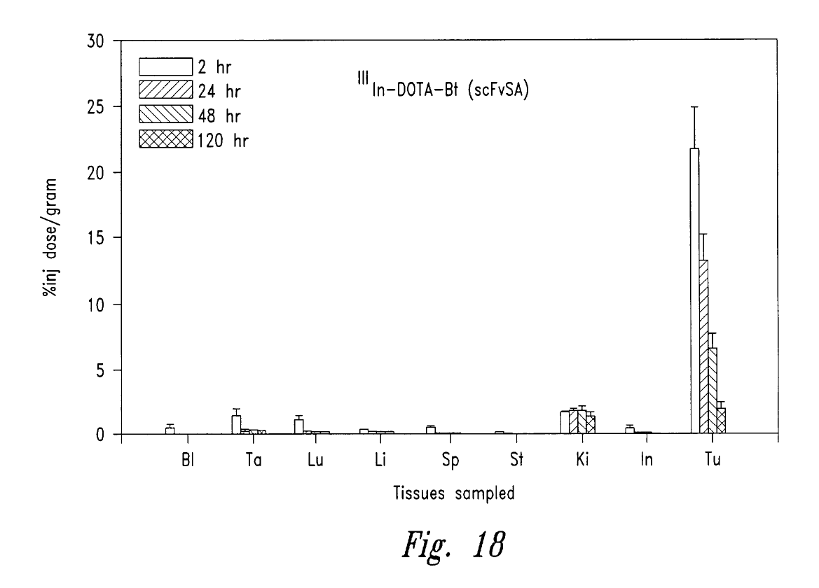

As shown in FIG. 18, the ¹¹¹In-DOTA-biotin radioactivity in blood and all non-xenograft soft tissues was below 2% of the injected dose/g. Further, ¹¹¹In-DOTA-biotin uptake and retention in liver is not seen, indicating that the fusion protein has been efficiently internalized by the liver, via the added clearing agent, and is unavailable to bind the subsequently administered radiobiotin. Stable delivery and retention of ¹¹¹In-DOTA-biotin at the tumor were observed. The highest concentration of radiobiotin at all time points was at the tumor (both stoichiometrically and relative to blood pool concentration). Peak concentrations of ¹¹¹In-DOTA-biotin at the tumor were 17-24% of injected dose/g (mean 21.66, s.d. 3.17). Tumor to blood ratios increased from about 90, 2 hours after DOTA-biotin injection, to greater than 700 by 24 hours. In these experiments no effort was made to optimize the dose of the fusion protein, clearing agent, or DOTA-biotin, nor was any effort made to optimize the schedule of administration of these components. (In FIG. 18, the tissues in order are blood, tail, lung, liver, spleen, stomach, kidney, intestine, and tumor.)

### Example XI

### Construction of Anti-TAG-72 CC49 Single Chain Antibody-Genomic Strepavidin Fusion

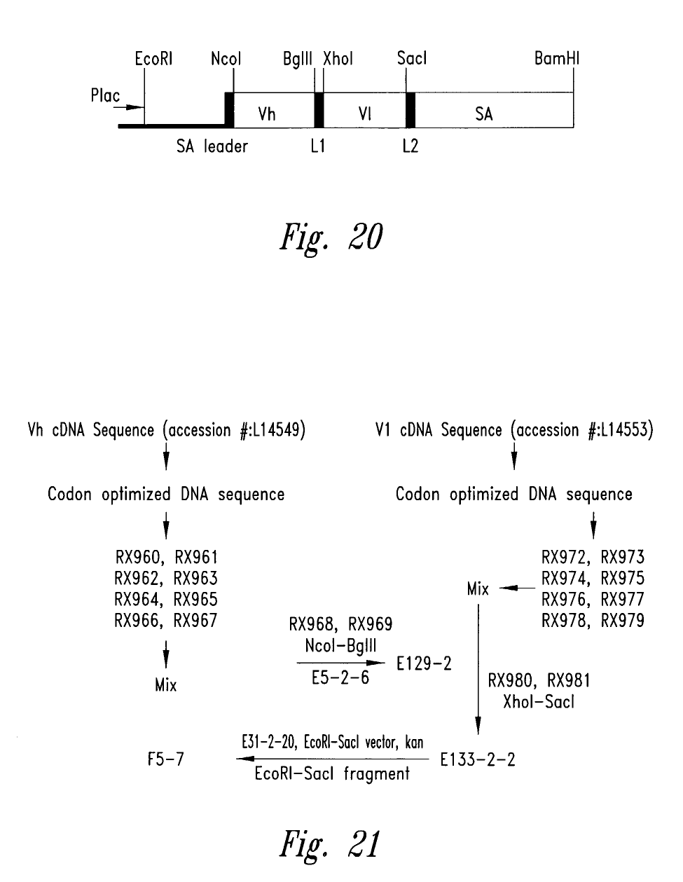

The murine CC49 single chain Fv/streptavidin (scFvSA) fusion protein is expressed from the genetic fusion of the single chain antibody of the variable regions (scFv) to the genomic streptavidin of Streptomyces avidinii. The scFv gene comprises the variable regions of the heavy (V_{H}) and light (V_{L}) chains separated by a DNA linker sequence (e.g., FIG. 20). The streptavidin coding sequence is joined to the 3′ terminus of the scFv gene, and the two genes are separated in-frame by a second DNA linker sequence. The signal sequence from the streptavidin gene is fused at the 5′ terminus of the scFvSA gene to direct expression to the E. coli periplasmic space. The scFvSA gene is under control of the lac promoter, and the expressed fusion protein is extracted and purified from E. coli and forms a soluble tetramer of about 176,000 molecular weight.

The cDNA sequences of the murine CC49 heavy chain (Vh) and light chain (Vl) were obtained from the Genbank database (accession numbers L14549 and L14553, respectively) and were further optimized based on E. coli codon usage. The scFvSA fusion gene consists of the Vh and Vl regions, which are separated by a 25-mer Gly₄Ser linker, fused to the genomic streptavidin-coding region.

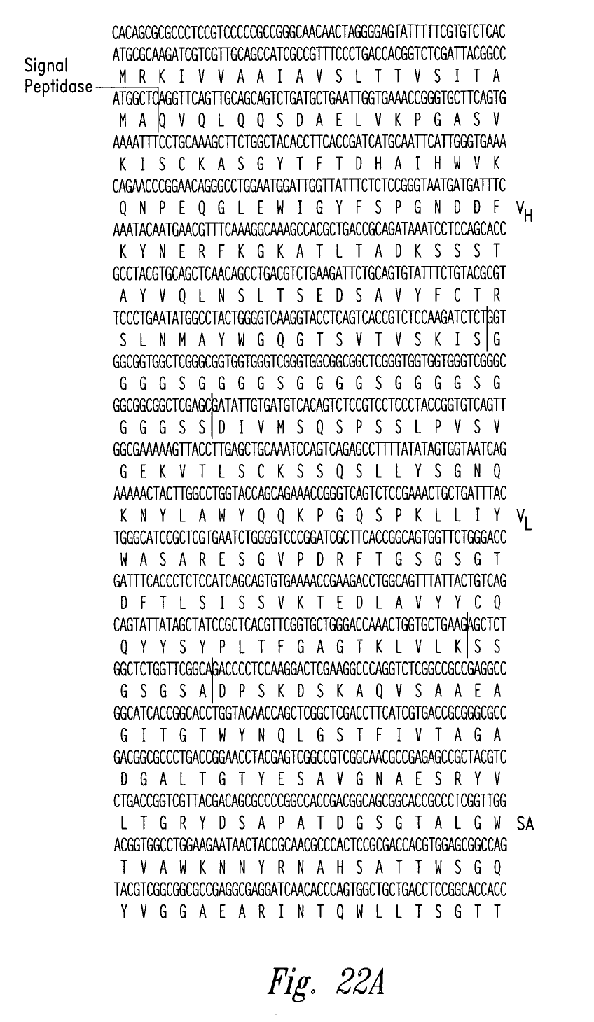

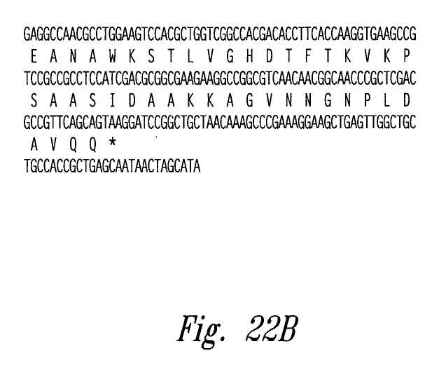

Pairwise oligos such as RX960-RX961, RX962-RX963, etc. (see list below) were annealed together using 5 cycles of the following polymerase chain reaction (PCR) protocol (95° C. for 45 sec; 50° C. for 45 sec; 74° C. for 1 min with Pfu polymerase). The products were passed through CentriSep columns (Princeton Separations) to desalt. Five μl of each of the PCR products were combined as templates, and 30 cycles of PCR were performed using RX968 and RX969 oligonucleotides for the heavy chain or RX980 and RX981 for the light chain. The PCR products were purified on a 1.5% agarose gel, and the DNAs were extracted. The PCR products were digested with restriction enzymes as indicated in FIG. 21 at 37° C. overnight, and the mixture was desalted on CentriSep columns. The heavy chain fragment was cloned in NcoI/BgIII-digested vector E5-2-6 to generate the E129-2, and the light chain fragment was cloned in XhoI/SacI-digested vector E129-2 to generate E133-2-2. The EcoRI-SacI fragment containing murine scFv of CC49 was excised from the E133-2-2 plasmid and cloned into E31-2-20 vector containing a kanamycin-resistant neo gene. The resultant F5-7 plasmid expressed the murine CC49 scFvSA fusion gene and exhibited kanamycin resistance when transformed into E. coli. The DNA and amino acid sequences of CC49 scFvSA (plasmid F5-7) are shown in FIG. 22.

The host organism is E. coli XL1-Blue, which has the genotype recA1 endA1 gyrA96 thi-1 hsdR17 supE44 relA1 lac [F′ proAB lacI^{q}ZΔM15 Tn10 (Tet^{r})]. The organism was purchased from Stratagene (La Jolla, Calif.), frozen at −70° C. as DNA competent cells, and the manufacturer's directions were followed to perform the transformation. Aliquots (50 μL) were plated on LB agar containing 50 μg per mL kanamycin, and were incubated for 2 days at 30° C. One colony was streaked for isolation on an LB plus kanamycin plate. The final construct is plasmid F5-7 in E. coli strain XL1-Blue.

All nucleotide primers, as listed below, were synthesized by Operon Technologies, Inc. (Alameda, Calif.).

| | |
|---|---|
| RX960 | |
| CAGGTTCAGT TGCAGCAGTC TGATGCTGAA TTGGTGAAAC CGGGTGCTTC AGTGAAAATT | (SEQ ID NO: 50) |
| RX961 | |
| TGCATGATCG GTGAAGGTGT AGCCAGAAGC TTTGCAGGAA ATTTTCACTG AAGCACCCGG | (SEQ ID NO: 51) |
| RX962 | |
| TACACCTTCA CCGATCATGC AATTCATTGG GTGAAACAGA ACCCGGAACA GGGCCTGGAA | (SEQ ID NO: 52) |
| RX963 | |
| TTTGAAATCA TCATTACCCG GAGAGAAATA ACCAATCCAT TCCAGGCCCT GTTCCGCGTT | (SEQ ID NO: 53) |
| RX964 | |
| CCGGGTAATG ATGATTTCAA ATACAATGAA CGTTTCAAAG GCAAACCCAC GCTGACCGCA | (SEQ ID NO: 54) |
| RX965 | |
| GCTGTTGAGC TGCACGTAGG CGGTGCTGGA GGATTTATCT GCGGTCAGCG TGGCTTTGCC | (SEQ ID NO: 55) |
| RX966 | |
| GCCTACGTGC AGCTCAACAG CCTGACGTCT GAAGATTCTG CAGTGTATTT CTCTACGCGT | (SEQ ID NO: 56) |
| RX967 | |
| GACTGAGGTA CCTTGACCCC AGTAGGCCAT ATTCAGGGAA CGCGTACAGA AATACACTGC | (SEQ ID NO: 57) |
| RX968 | |
| GAATTCCCAT GGCTCAGGTT CAGTTGCAGC AGTCT | (SEQ ID NO: 58) |
| RX969 | |
| CACCAGAGAT CTTGGAGACG GTGACTGAGG TACCTTGACC CCA | (SEQ ID NO: 59) |
| RX972 | |
| GATATTGTGA TGTCACAGTC TCCGTCCTCC CTACCGGTGT CAGTTCCCGA AAAAGTTACC | (SEQ ID NO: 60) |
| RX973 | |
| ACCACTATAT AAAAGGCTCT GACTGGATTT GCACCTCAAG GTAACTTTTT CGCCAACTGA | (SEQ ID NO: 61) |
| RX974 | |
| CAGAGCCTTT TATATAGTGG TAATCAGAAA AACTACTTGG CCTGGTACCA GCAGAAACCG | (SEQ ID NO: 62) |
| RX975 | |
| AGCGGATGCC CACTAAATCA GCAGTTTCGG AGACTGACCC CGTTTCTGCT GGTACCAGGC | (SEQ ID NO: 63) |
| RX976 | |
| CTGATTTACT GGGCATCCGC TCGTGAATCT GGGGTCCCGG ATCGCTTCAC CGGCAGTGGT | (SEQ ID NO: 64) |
| RX977 | |
| TTTCACACTG CTGATGGAGA GGGTGAAATC GGTCCCAGAA CCACTGCCGG TGAAGCGATC | (SEQ ID NO: 65) |
| RX978 | |
| CTCTCCATCA GCAGTGTGAA AACCGAAGAC CTGGCAGTTT ATTACTGTCA GCAGTATTAT | (SEQ ID NO: 66) |
| RX979 | |
| CACCAGTTTG GTCCCAGCAC CGAACGTGAG CGGATAGCTA TAATACTGCT GACAGTAATA | (SEQ ID NO: 67) |
| RX980 | |
| CGGCGGCTCG AGCGATATTG TGATGTCACA GTCT | (SEQ ID NO: 68) |
| RX981 | |
| GAGCCAGAGC TCTTCAGCAC CAGTTTGGTC CCAGCACC | (SEQ ID NO: 69) |

### Example XII

### Expression and Purification of CC49 scFvSA Protein

Transformants of E. coli strain XL1-Blue containing plasmid F5-7 (CC49 scFvSA) were grown overnight at 30° C. in Terrific broth (20 ml; Sigma) containing kanamycin (50 μg/ml). The culture was diluted 100-fold into fresh medium and grown in a shaking incubator at 30° C. When the culture attained an A₆₀₀ of 0.3-0.5, IPTG (Amersham Pharmacia Biotech, Piscataway, N.J.) was added to a final concentration of 0.2 mM, and incubation was continued overnight. Periplasmic extracts were prepared for qualitative analysis of the scFvSA expression level. Cells were resuspended in an ice-cold solution of 20% sucrose, 2 mM EDTA, 30 mM Tris, (pH 8.0), and lysozyme (2.9 mg/ml) and were incubated on ice for 30 min. Supernatants were analyzed on 4-20% Tris-glycine SDS-PAGE gels (Novex) under non-reducing, non-boiled conditions, and gels were stained with Coomassie Blue.

Clones were further grown in an 8L fermentor and analyzed for expression level. The primary inoculum (50 ml) was grown overnight at 30° C. in shake flasks containing Terrific broth plus 50 μg/ml kanamycin. The culture was then diluted 100-fold into the same medium and grown at 30° C. for an additional 4-5 h. This secondary inoculum (0.5 liter) was transferred to a 14 liter BioFlo 3000 fermentor (New Brunswick Scientific) containing 8 liters of complete E. coli medium (essentially as previously described above with respect to B9E9 scFvSA). Cells were harvested at 48 h post-inoculation in a continuous flow centrifuge (Pilot Powerfuge, Carr Separations, Franklin, Mass.), washed with PBS (10 mM sodium phosphate, 150 mM NaCl, pH 7.2), and pelleted by centrifugation. A typical fermentation produced 80-90 g of cells (wet wt) per liter culture medium.

For determining expression levels, cells were washed twice in PBS, resuspended to the original volume, and disrupted either by sonication on ice (Branson Ultrasonics, Danbury, Conn.) or through two cycles of microfluidization (Microfluidics International, Newton, Mass.). A rhodamine-biotin HPLC assay was used for quantitating fusion protein in the supernatent of a centrifuged sample of crude lysate, as described previously as described previously in Example VI, above and in Schultz et al., Cancer Res. 60:6663-9, 2000). The concentration of fusion protein in the crude lysate was calculated by comparison to a standard analyzed under the same conditions. The molar extinction coefficient for the fusion protein standard was calculated using a previously described method summing the relative contributions of amino acids absorbing at 280 nm (Gill and von Hippel, Analyt. Chem. 182:319-326, 1989). Expression levels of CC49 scFvSA in fermentor-grown cells were 100-130 mg/liter.

The iminobiotin affinity matrix was prepared as described previously in Example VI, above and in Schultz et al., Cancer Res 60:6663-9, 2000. The fusion protein was purified from E. coli cells (650-750 g) by iminobiotin affinity chromatography as described above.

Typical recoveries from iminobiotin chromatography were 50-60% with less than 5% appearing in the flow-through and wash. The residual remained as aggregate/entrapped material on the column.

### Example XIII

### Biochemical Characterization of CC49 scFvSA Protein

The CC49 scFvSA is secreted into the periplasm of genetically engineered E. coli as monomeric subunits (43,952 Daltons) that spontaneously fold into a tetrameric protein with a molecular weight of 175,808 Daltons. The tetrameric fusion protein contains four antigen binding sites and four biotin binding sites.

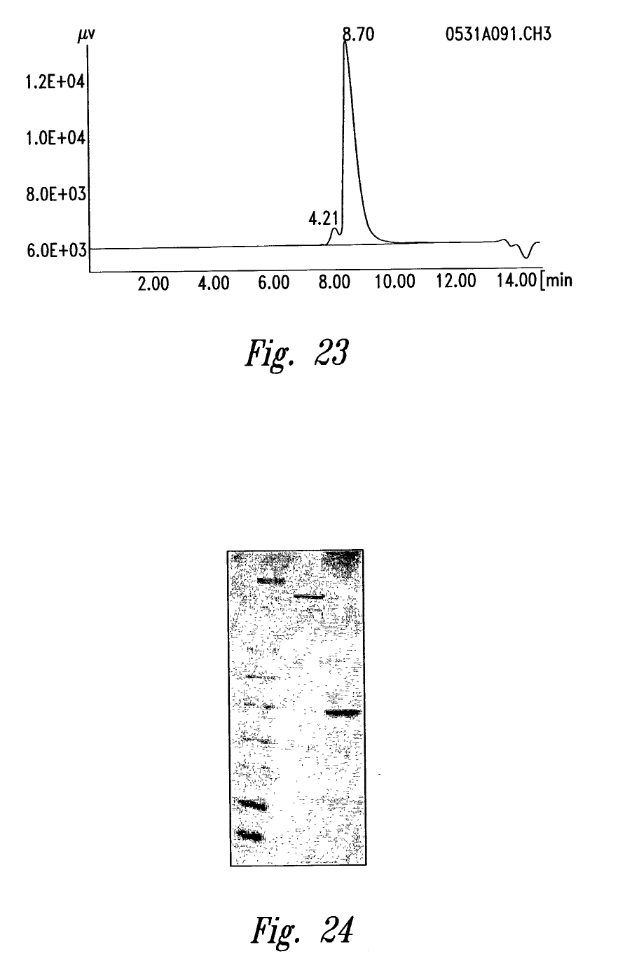

Size exclusion HPLC. Purified protein preparations were analyzed by size exclusion HPLC performed on a Zorbax GF-250 column with a 20 mM sodium phosphate/0.5 M NaCl mobile phase. The eluent is monitored at 254 nm. FIG. 23 shows the HPLC chromatogram of iminobiotin-purified CC49 scFvSA. The peak at retention time 8.70 minutes is the tetrameric fusion protein with a 5% aggregate eluting at 8.21 minutes. This analysis demonstrated that all of the purified protein was tetrameric or an aggregate thereof.

SDS-PAGE Analysis. Purified CC49 scFvSA was analyzed on 4-20% Tris-glycine SDS-PAGE gels (Novex, San Diego, Calif.) under nonreducing conditions. Before electrophoresis, samples were mixed with SDS-loading buffer and incubated at either room temperature or 95° C. for 5 min. Gels were stained with Coomassie blue.

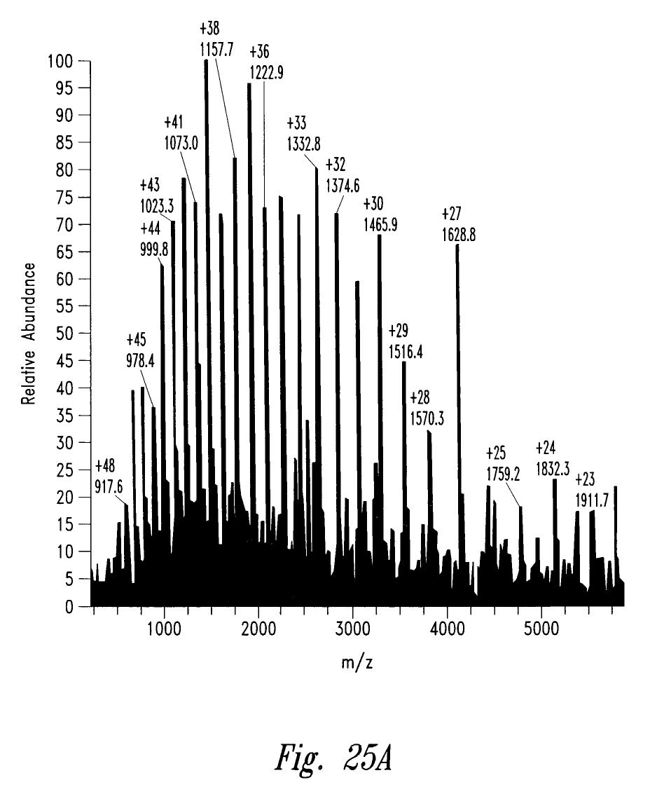

SDS-PAGE demonstrated that the fusion protein was purified to >95% homogeneity after iminobiotin chromatography (FIG. 24, lane 2). The major band migrated at the expected molecular weight of ˜176 kDa with minor isoforms evident. However, all bands resolved into a single species of ˜44 kDa when the protein was boiled prior to electrophoresis, consistent with a single protein entity dissociable into its homogeneous subunit (FIG. 24, lane 3).

Molecular weight determination. Liquid chromatographic separation was conducted with an Hewlett Packard series 1100 system, fitted with a Jupiter C18 column (300 Å, 3.2×50 mm, 5μ) and C18 “SafeGuard” column (Phenomenex, Torrance, Calif.) at a flow rate of 500 μl/min. The mobile phase was composed of water/1% formic acid (buffer A) and acetonitrile/1% formic acid (buffer B). The gradient applied was 2% B for 3 min rising to 99% B within 7 min. CC49 scFvSA (10 μl) was eluted at a retention time of 6.8 min. The analytical column was interfaced with a Thermoquest/Finnigan ESI LCQ ion trap mass spectrometer (San Jose, Calif.). The instrument was calibrated with myoglobin and operated in the positive ion mode with the heated capillary set to 200° C. and 5.1 kV applied to the electrospray needle. The data were acquired in a full scan MS mode (m/z [500-2000 Da/z]) using automated gain control with 3 microscans and a maximum ion time of 500 ms, performed essentially as described in Example VII above.

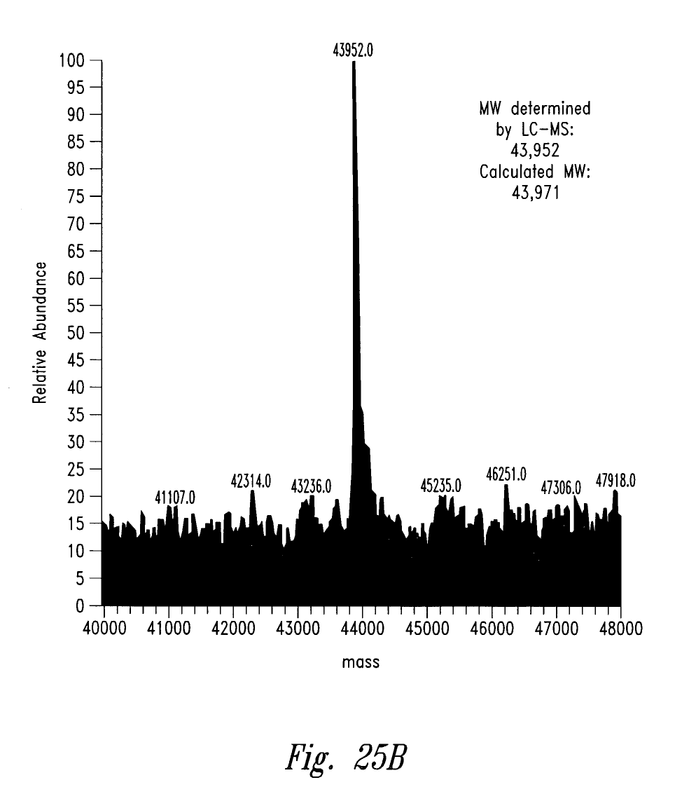

The mass spectrum of the CC49 monomer showed a deconvoluted molecular weight of 43,952, which is in agreement with the calculated most abundant mass of 43,971 (FIG. 25).

Amino-terminal sequencing. Automated amino acid sequencing was performed using a Procise 494 sequenator (Applied Biosystems, Inc., Foster City, Calif.). This revealed that the leader sequences of CC49 scFvSA were cleaved at the expected signal peptidase site adjacent to the first amino acid of the heavy chain variable region.

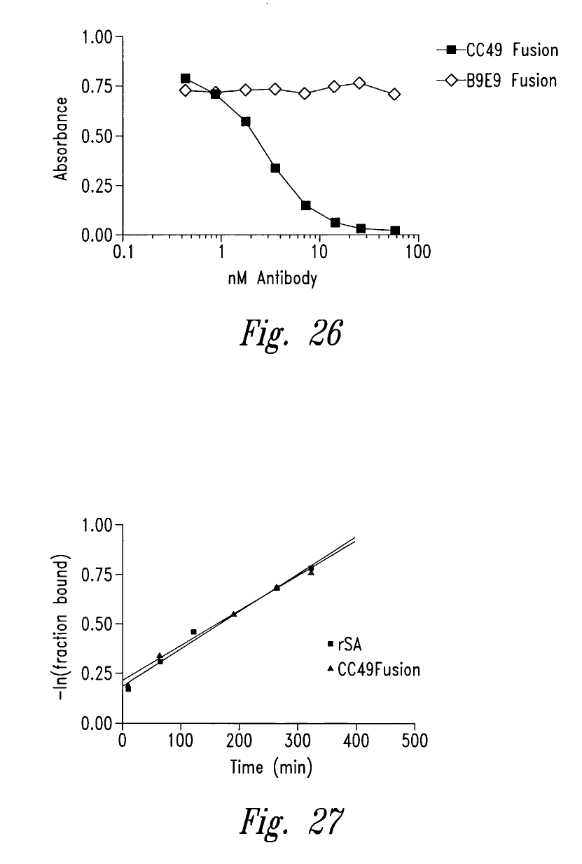

Competitive Immunoreactivity ELISA. Serial dilutions of the anti-TAG-72 CC49 scFvSA or a control anti-CD20 B9E9 scFvSA were allowed to compete with horseradish peroxidase-labeled murine CC49 whole antibody for binding to bovine submaxillary mucin (Sigma Chemical, St. Louis, Mo.), which is a source of TAG-72 antigen. Binding of CC49 scFvSA was specific, and the curve exhibited the sigmoidal shape consistent with a one-site competition model (FIG. 26).

Biotin-Binding Capacity. Biotin binding capacity was determined by incubation of a known quantity of fusion protein with a 9-fold molar excess of [³H]biotin (NEN Research Products, Boston, Mass.). After removal of uncomplexed biotin using streptavidin-immobilized beads (Pierce Chemical; Rockford, Ill.), the amount of [³H]biotin associated with the fusion protein was determined.

CC49 scFvSA was capable of binding an average of 3.7 biotins as compared + to 4.0 biotin-binding sites for recombinant streptavidin.

Biotin Dissociation Rate. The rate of biotin dissociation was determined at 37° C. in 0.25 M sodium phosphate, 0.15 M NaCl, 0.25% bovine serum albumin (pH 7.0) containing 10 μM CC49 scFvSA or recombinant streptavidin (control), 0.06 μM [³H]biotin (58 mCi/μmole) and 30 mM ascorbate as [³H]biotin stabilizer. After incubation to reach equilibrium, biocytin (4 mM) was added to initiate irreversible dissociation of [³H]biotin. Aliquots were withdrawn periodically and diluted 20-fold in phosphate-buffered saline containing 0.5% bovine serum albumin. The samples were split for assessment of “total” and “free” [³H]biotin, the latter after protein precipitation using zinc sulfate/NaOH (60 μM each) added sequentially. Radioactivity was assessed in a fluoroscintillate using a Hewlett Packard beta counter. Linear regression analysis of a plot of the In (fraction bound) versus time yielded a dissociation rate constant. The biotin dissociation rate of CC49 scFvSA was identical to that of recombinant streptavidin (r-SA) (T_{1/2}=397 min and 371 min, respectively), indicating fully functional biotin binding (FIG. 27). The above experiments were performed essentially the same as set forth in Example VII, above.

### Example XIV

### Interaction of CC49 scFvSA With Clearing Agent

To function in the Pretarget® regimen, CC49 scFvSA in circulation must complex efficiently with the synthetic clearing agent (sCA) and be removed rapidly from circulation by subsequent uptake into the liver via the Ashwell receptors. Reactivity of CC49 scFvSA with sCA was assessed in a non-tumored mouse model (n=3/group) where ¹²⁵I-CC49 scFvSA (600 μg) was injected i.v. at t=0, followed 18 hours later by a single i.v. bolus injection of approximately a 20-fold stoichiometric excess of sCA.

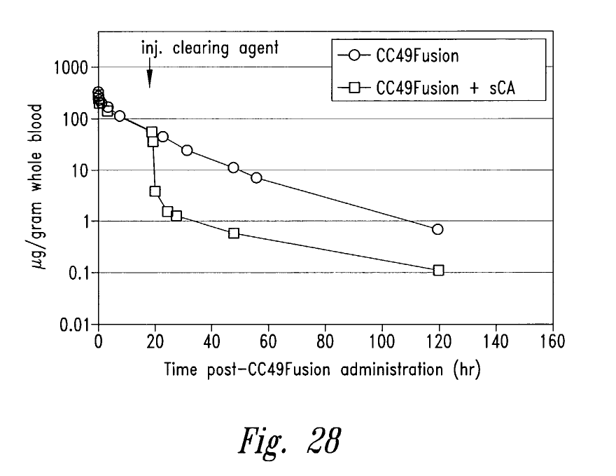

FIG. 28 shows that CC49 scFvSA was rapidly removed from the blood by sCA. The first 2 hours after sCA administration are characterized by a very rapid decline in serum CC49 scFvSA concentration, followed by resumption of its initial, pre-sCA rate. This is consistent with prior results utilizing the sCA with a variety of streptavidin-containing constructs.

### Example XV

### Biodistribution of ¹¹¹In-DOTA-Biotin After Pretargeting With CC49 scFvSA

The expressed CC49 scFvSA gene fusion was tested in a full pretarget protocol, essentially as described above in Example VIII except that the tests were performed in female nude mice bearing TAG-72 antigen-positive LS-174T human colon cancer xenografts (100-300 mm³), subcutaneously implanted on the right flank. In these experiments, 600 μg of ¹²⁵I-labeled fusion protein was injected intravenous (iv) and allowed to circulate for 20 hours prior to iv injection of 100 μg of synthetic clearing agent (sCA) (See e.g., PCT Publication Nos. WO 97/46098 and WO 95/15978). Four hours after the sCA injection, there was an injection of 1.0 μg of ¹¹¹In-DOTA-biotin, essentially a chelating agent containing a radionuclide, conjugated to biotin (see U.S. Pat. Nos. 5,578,287 and 5,608,060). Mice were sampled for blood, then sacrificed and dissected at 26, 48, 72, and 144 hours after injection of CC49 scFvSA.

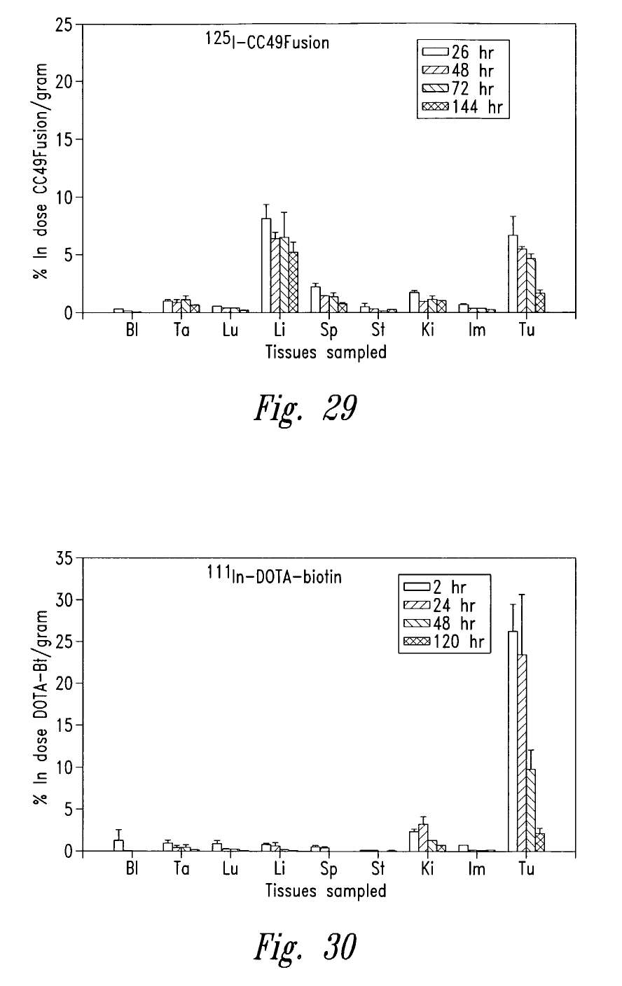

The concentration of ¹²⁵I-CC49 scFvSA radioactivity in blood and most well-perfused soft tissues is very low, due to the low blood pool concentration induced by the sCA complexation and subsequent hepatic clearance (FIG. 29). (The tissues in order in FIGS. 29 and 30 are blood, tail, lung, liver, spleen, stomach, kidney, intestine, and tumor.) The exceptions were liver and tumor. Liver uptake and retention of fusion protein is due to the mechanism of clearing agent action. The ¹²⁵I-CC49 scFvSA exhibits evidence of in vivo immunoreactivity by the retention of relatively high radiolabel concentration at the tumor (both stoichiometrically and relative to blood pool concentration) at all time points.

The concentration of pretargeted ¹¹¹In-DOTA-biotin radioactivity in blood and all non-xenograft soft tissues is very low (FIG. 30). Despite the high concentrations of fusion protein in the liver noted above, ¹¹¹In-DOTA-biotin uptake and retention in this organ is not in evident, indicating that the fusion protein has been efficiently internalized and is unavailable to bind the subsequently administered radiobiotin. The highest concentration of ¹¹¹In-DOTA-biotin is at the tumor at all time points. The rapid uptake, achieving peak concentrations at the earliest time point sampled is a hallmark of pretargeting. Stable delivery and retention of ¹¹¹In-DOTA-biotin at the tumor is also observed. Peak concentrations of ¹¹¹In-DOTA-biotin at the tumor are 22-28% injected dose/g and occurred within 2 hours post administration of the DOTA-biotin. Tumor-to-blood ratios increased from ca. 40, 2 hours post administration, to >1600 by 24 hours. The area under the curve for blood was 28, while the area under the curve for tumor was 1394, resulting in a high specificity index of 49.

### Example XVI

### Construction of Anti-CD25 (Anti-TAC) Single Chain Antibody-Genomic Streptavidin Fusion

The murine anti-CD25 single chain Fv/streptavidin (scFvSA) fusion protein is expressed from the genetic fusion of the single chain antibody of the variable regions (scFv) to the genomic streptavidin of Streptomyces avidinii. The scFv gene consists of the variable regions of the heavy (V_{H}) and light (V_{L}) chains separated by a DNA linker sequence. The streptavidin coding sequence is joined to the 3′ terminus of the scFv gene, and the two genes are separated in-frame by a second DNA linker sequence. The signal sequence from the streptavidin gene is fused at the 5′ terminus of the scFvSA gene to direct expression to the E. coli periplasmic space. The scFvSA gene is under control of the lac promoter, and the expressed fusion protein is extracted and purified from E. coli and forms a soluble tetramer of about 172,000 molecular weight.

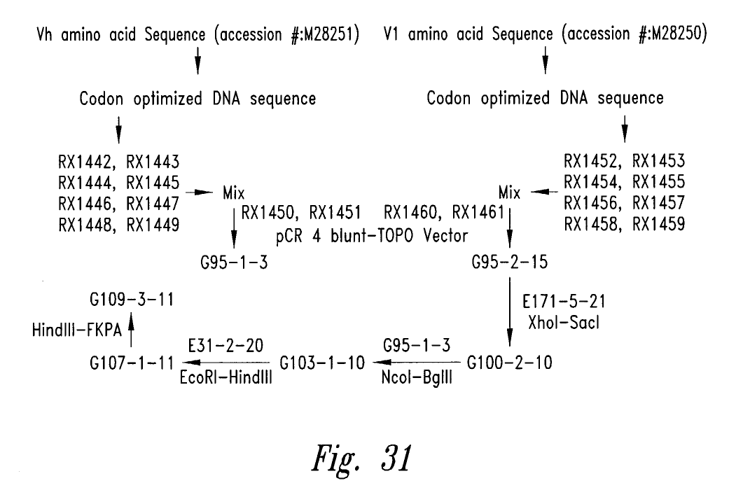

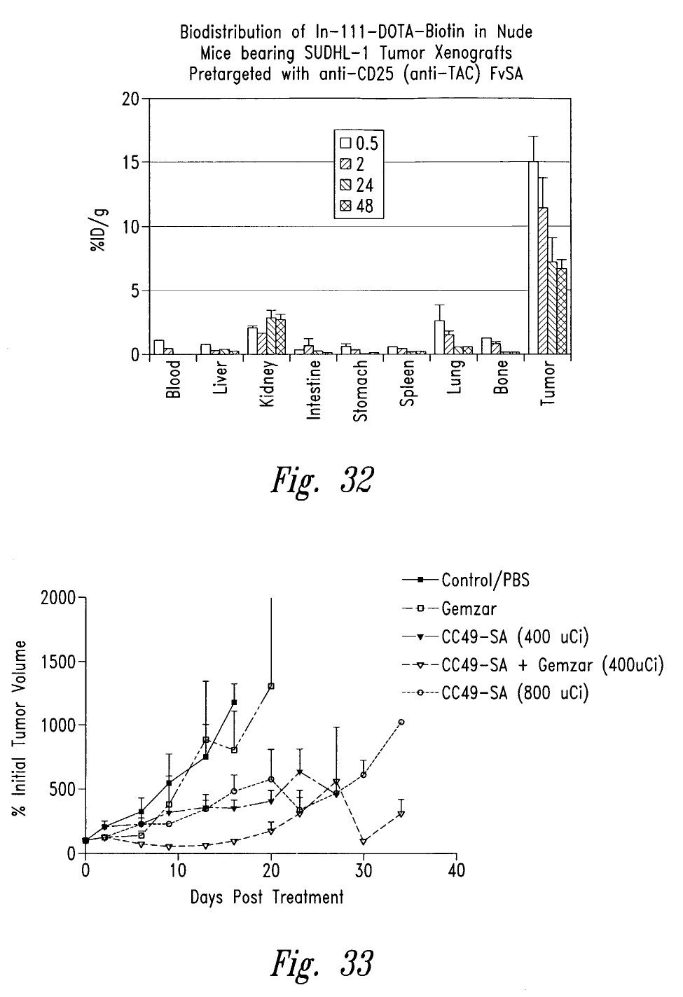

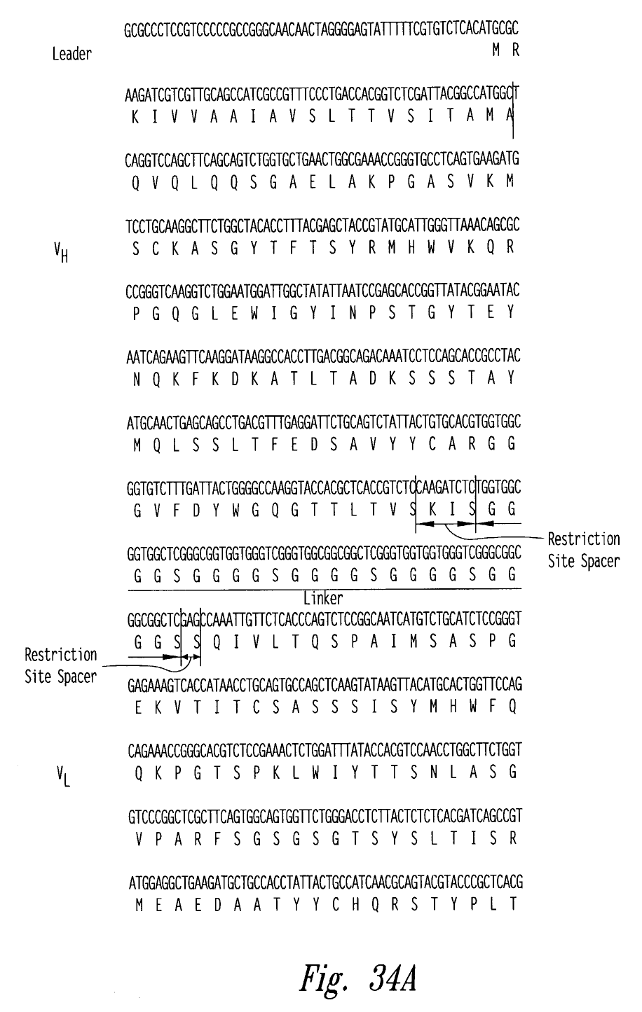

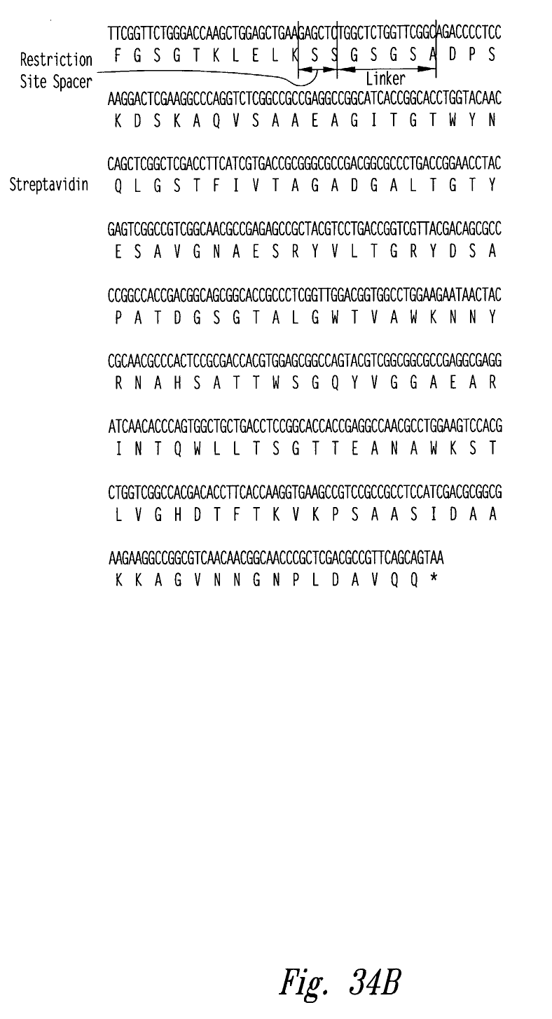

The cDNA sequences of the murine anti-CD25 heavy chain (V_{H}) (Genbank accession numbers M28251, SEQ ID NO: 85 and SEQ ID NO: 91) and light chain (V_{L}) (Genbank accession numbers M28250, SEQ ID NO: 86 and SEQ ID NO: 92) and were further optimized based on E. coli codon usage, according to the following procedure. The scFvSA fusion gene consists of the V_{H} and V_{L} regions, separated by a 25-mer Gly₄Ser linker, fused to the genomic streptavidin-coding region. Pairwise oligos, for example, RX1442 plus RX1443 and RX1444 plus RX1445 were annealed together using 5 cycles of the following polymerase chain reaction (PCR) protocol (95° C. for 30 sec; 50° C. for 30 sec; 74° C. for 1 min with Pfu polymerase). The products were passed through CentriSep columns (Princeton Separations) to desalt. Five μl of each of the PCR products were combined as templates, and 35 cycles of PCR were performed using RX1450 and RX1451 oligonucleotides for the heavy chain or RX1460 and RX1461 for the light chain. The PCR products were purified on a 1.5% agarose gel, and the DNAs were extracted. The purified PCR products were treated with Taq polymerase for 30 min at 72° C. in the presence of dNTP nucleotides, and the mixtures were passed through CentriSep columns to desalt. The Taq-treated PCR products were cloned in a pCR4 blunt TOPO vector (Invitrogen, Sorrento Valley, Calif.) to generate G95-1-3 containing the V_{H} fragment and G95-2-15 containing the V_{L} fragment, respectively. A XhoI-SacI fragment from G95-2-15 was excised and cloned into E171-5-21 vector containing an ampicillin-resistant gene to generate G100-2-10. A NcoI-BgIII fragment from G95-1-3 was isolated and cloned into G100-2-10 previously digested with NcoI and BgIII. The resulting plasmid G103-1-10 expressed the murine anti-Tac scFvSA fusion protein and exhibited ampicillin resistance when transforming E. coli (XL1-Blue). The plasmid G103-1-10 was further converted into a kanamycin-resistant vector to produce construct G107-1-11. Plasmid G107-1-11 was further derivatized into plasmid G109-3-11, which contains the chaperone gene FkpA. The preparation of plasmid G109-3-11 is schematically presented in FIG. 31. The DNA (SEQ ID NO: 87) and amino acid (SEQ ID NO: 88) sequences of anti-TAC scFvSA (plasmid G103-1-10 and G107-1-11) are disclosed herein (FIG. 34). Further, amino acid residues 1-22 (nucleotides 55-120) represent the N-terminal signal sequence of this particular construct. However, it is also appreciated that additional modification and/or optimization of a particular signal sequence associated with a anti-TAC scFvSA, or similar construct, may be performed as necessary, as would be readily understood by those of ordinary skill in the art.

The host organism is E. coli XL1-Blue, which has the genotype recA1 endA1gyrA96 thi-1 hsdR17 supE44 relA1 lac [F′ proAB lacI^{q}ZΔM15 Tn10 (Tet^{r})]. The organism was purchased from Stratagene (La Jolla, Calif.), frozen at −70° C. as DNA competent cells, and the manufacturer's directions were followed to perform the transformation. Aliquots (50 μL) were plated on LB agar containing 50 μg per mL kanamycin or ampicillin, and were incubated for 2 days at 30° C. Colonies were streaked for isolation on plates containing the appropriate antibiotic. Constructs are plasmid G103-1-10 (ampicillin-resistant) and G107-1-11 (kanamycin-resistant) in E. coli strain XL1-Blue.

All nucleotide primers, as listed below, were synthesized by Operon Technologies, Inc. (Alameda, Calif.).

| | |
|---|---|
| RX1442 | |
| CAGGTCCAGC TTCAGCAGTC TGGTGCTGAA CTGGCGAAAC CGGGTGCCTC AGTGAAGATG | (SEQ ID NO: 70) |
| RX1443 | |
| ACGGTAGCTC GTAAAGGTGT AGCCAGAAGC CTTGCAGGAC ATCTTCACTG AGGCACCCGG | (SEQ ID NO: 71) |
| RX1444 | |
| TACACCTTTA CGAGCTACCG TATGCATTGG GTTAAACAGC GCCCGGGTCA AGGTCTGGAA | (SEQ ID NO: 72) |
| RX1445 | |
| TTCCGTATAA CCGGTGCTCG GATTAATATA GCCAATCCAT TCCAGACCTT GACCCGGGCG | (SEQ ID NO: 73) |
| RX1446 | |
| CCGAGCACCG GTTATACGGA ATACAATCAG AAGTTCAAGG ATAAGGCCAC CTTGACGGCA | (SEQ ID NO: 74) |
| RX1452 | |
| CAAATTGTTC TCACCCAGTC TCCGGCAATC ATGTCTGCAT CTCCGGGTGA GAAAGTCACC | (SEQ ID NO: 75) |
| RX1453 | |
| GTGCATGTAA CTTATACTTG AGCTGGCACT GCAGGTTATG GTGACTTTCT CACCCGGAGA | (SEQ ID NO: 76) |
| RX1454 | |
| TCAAGTATAA GTTACATGCA CTGGTTCCAG CAGAAACCGG GCACGTCTCC CAAACTCTGG | (SEQ ID NO: 77) |
| RX1455 | |
| AGCCGGGACA CCAGAAGCCA GCTTGGACGT CGTATAAATC CAGACTTTCG GAGACGTGCC | (SEQ ID NO: 78) |
| RX1456 | |
| CTGGCTTCTG GTGTCCCGGC TCGCTTCAGT GGCAGTGGTT CTGGGACCTC TTACTCTCTC | (SEQ ID NO: 79) |
| RX1457 | |
| ATAGGTGGCA GCATCTTCAG CCTCCATACG GCTGATCGTG AGAGAGTAAG AGGTCCCAGA | (SEQ ID NO: 80) |
| RX1458 | |
| GCTGAAGATG CTGCCACCTA TTACTGCCAT CAACGCAGTA CGTACCCGCT CACGTTCGGT | (SEQ ID NO: 81) |
| RX1459 | |
| TTCAGCTCCA GCTTGGTCCC AGAACCGAAC GTGAGCGGGT ACGT | (SEQ ID NO: 82) |
| RX1460 | |
| CGGCGGCTCG AGCCAAATTG TTCTCACCCA GTCT | (SEQ ID NO: 83) |
| RX1461 | |
| CCACCAGAGC TCTTCAGCTC CAGCTTGGTC CC | (SEQ ID NO: 84) |
| RX1450 | |
| GAATTCCCAT GCCTCAGGTC CAGCTTCAGC AGTCT | (SEQ ID NO: 89) |
| RX1451 | |
| CACCAGAGAT CTTGGAGACG GTGAGCGTGG TACCTTCGCC CCAGTA | (SEQ ID NO: 90) |

### Example XVII

### Expression and Purification of Anti-CD25 (Anti-TAC) scFvSA Protein

Transformants of E. coli strain XL1-Blue containing plasmid G103-1-10 (anti-CD25 scFvSA) were grown overnight at 30° C. in Terrific broth (20 ml; Sigma) containing ampicillin (50 μg/ml). The culture was diluted 100-fold into fresh medium and grown in a shaking incubator at 30° C. When the culture attained an A₆₀₀ of 0.3-0.5, IPTG (Amersham Pharmacia Biotech, Piscataway, N.J.) was added to a final concentration of 0.2 mM, and incubation was continued overnight. Periplasmic extracts were prepared for qualitative analysis of the scFvSA expression level. Cells were resuspended in an ice-cold solution of 20% sucrose, 2 mM EDTA, 30 mM Tris, (pH 8.0), and lysozyme (2.9 mg/ml) and were incubated on ice for 30 min. Supernatants were analyzed on 4-20% Tris-glycine SDS-PAGE gels (Novex) under non-reducing, non-boiled conditions, and gels were stained with Coomassie Blue.

Clones were further grown in an 8L fermentor and analyzed for expression level. The primary inoculum (50 ml) was grown overnight at 30° C. in shake flasks containing Terrific broth plus 50 μg/ml ampicillin, or suitable media known to those of ordinary skill in the art. The culture was then diluted 100-fold into the same medium and grown at 30° C. for an additional 4-5 h. This secondary inoculum (0.5 liter) was transferred to a 14 liter BioFlo 3000 fermentor (New Brunswick Scientific) containing 8 liters of complete E. coli medium. Cells were harvested at 48 h post-inoculation in a continuous flow centrifuge (Pilot Powerfuge, Carr Separations, Franklin, Mass.), washed with PBS (10 mM sodium phosphate, 150 mM NaCl, pH 7.2), and pelleted by centrifugation. A typical fermentation produced 80-90 g of cells (wet wt) per liter culture medium.

For determining expression levels, cells were washed twice in PBS, resuspended to the original volume, and disrupted either by sonication on ice (Branson Ultrasonics, Danbury, Conn.) or through two cycles of microfluidization (Microfluidics International, Newton, Mass.). As described in Example XII, a rhodamine-biotin HPLC assay was used for quantitating fusion protein in the supernatant of a centrifuged sample of crude lysates. The concentration of fusion protein in the crude lysate was calculated by comparison to a standard analyzed under the same conditions. The molar extinction coefficient for the fusion protein standard was calculated using a previously described method summing the relative contributions of amino acids absorbing at 280 nm (Gill and von Hippel, Analyt. Chem. 182:319-326, 1989). Expression levels of anti-TAC scFvSA in fermentor-grown cells were about 110 mg/liter.

The iminobiotin affinity matrix was prepared as described Example VI. The fusion protein was purified from E. coli cells (650-750 g) by iminobiotin affinity chromatography as described in Example XII.

Typical recoveries from iminobiotin chromatography were about 80% with less than 5% appearing in the flow-through and wash. The residual remained as aggregate/entrapped material on the column.

### Example XVIII

### Biochemical Characterization of Anti-CD25 (Anti-TAC) scFvSA Protein

The anti-CD-25 scFvSA is secreted into the periplasm of genetically engineered E. coli as monomeric subunits (43,098 Daltons) that spontaneously fold into a tetrameric protein with a molecular weight of 172,392 Daltons. The tetrameric fusion protein contains four antigen-binding sites and four biotin-binding sites.

Size exclusion HPLC. Purified protein preparations were analyzed by size exclusion HPLC performed on a Zorbax GF-250 column with a 20 mM sodium phosphate/0.5 M NaCl mobile phase. The eluent is monitored at 254 nm and the peak at retention time 7.70 minutes contains the tetrameric fusion protein with a 5% aggregate eluting at 7.13 minutes.

SDS-PAGE Analysis. Purified anti-TAC scFvSA was analyzed on 4-20% Tris-glycine SDS-PAGE gels (Novex, San Diego, Calif.) under nonreducing conditions. Before electrophoresis, samples were mixed with SDS-loading buffer and incubated at either room temperature or 95° C. for 5 min. Gels were stained with Coomassie blue. Accordingly, SDS-PAGE demonstrates that the fusion protein was purified to >95% homogeneity after iminobiotin chromatography. The major band migrated at the expected molecular weight of ˜172 kDa with minor isoforms evident.

Molecular weight determination. For mass analysis of monomeric anti-TAC, liquid chromatographic separation was conducted with a Hewlett Packard series 1100 system fitted with a PolyHYDROXYETHYL aspartamide column (200×9.4 mm, 5μ, 1000 Å, PolyLC Inc., Columbia, Md.) operated in the size exclusion mode. The mobile phase, composed of water containing 50 mM formic acid, was introduced at a flow rate of 1 ml/min, of which 3 parts were directed to the HP1100 UV detector, and one part to an ESI LCQ ion trap mass spectrometer (Thermo Finnigan, San Jose, Calif.). Using a “tee” connector, the LC effluent directed to the mass spectrometer was combined with a solution of acetonitrile containing 50 mM formic acid introduced at a flow rate of 100 μl/min. The acetonitrile was infused using a Graseby 3400 syringe pump (Graseby Medical Limited, Watford, UK). The mass spectrometer was calibrated with myoglobin and operated in the positive ion mode with the heated capillary set to 175° C. and 4.5 kV applied to the electrospray needle. The data were acquired in a full scan MS mode with an acquisition range of m/z 800-2000. The total ion current showed one peak eluting at a retention time of 6.8 minutes. Concurrent UV analysis showed absorption of the eluent at 278 nm. The mass spectrum exhibited an envelope of ions charged with 22 to 43 protons within a mass range of 1000-2000 m/z. Using Xcalibur software (Thermo Finnigan), the ion envelope was deconvoluted to obtain a mass of Mᵣ 43,078±0.05% (±21.5), which is in agreement with the calculated average mass Mᵣ 43,098 of anti-TAC.

Immunoreactivity Assay. Immunoreactivity was evaluated using SUDHL-1, an anaplastic large cell lymphoma cell line that expresses CD25 on the cell surface. A constant concentration of ¹²⁵I-labeled fusion protein (5 ng) or unmodified HAT mAb (5 ng) was incubated with an increasing number of SUDHL-1 cells in microcentrifuge tubes for 1 hour at 4° C. After centrifugation, the cell pellet was counted using a gamma counter and the binding was calculated. The anti-CD25 scFvSA and HAT mAb bound to the CD25-positive SUDHL-1 cells. Maximal binding of radiolabeled fusion protein and HAT were 85% and 78%, respectively.

Biotin-Binding Capacity. Biotin binding capacity was determined by incubation of a known quantity of fusion protein with a 9-fold molar excess of [³H]biotin (NEN Research Products, Boston, Mass.). After removal of uncomplexed biotin using streptavidin-immobilized beads (Pierce Chemical; Rockford, Ill.), the amount of [³H]biotin associated with the fusion protein was determined.

Anti-TAC scFvSA was capable of binding an average of 3.5 biotins as compared to 4.0 biotin-binding sites for recombinant streptavidin.

Biotin Dissociation Rate. The rate of biotin dissociation was determined at 37° C. in 0.25 M sodium phosphate, 0.15 M NaCl, 0.25% bovine serum albumin (pH 7.0) containing 10 μM anti-CD25 scFvSA or recombinant streptavidin (control), 0.06 μM [³H]biotin (58 mCi/μmole) and 30 mM ascorbate as [³H]biotin stabilizer. After incubation to reach equilibrium, biocytin (4 mM) was added to initiate irreversible dissociation of [³H]biotin. Aliquots were withdrawn periodically and diluted 20-fold in phosphate-buffered saline containing 0.5% bovine serum albumin. The samples were split for assessment of “total” and “free” [³H]biotin, the latter after protein precipitation using zinc sulfate/NaOH (60 μM each) added sequentially. Radioactivity was assessed in a fluoroscintillate using a Hewlett Packard beta counter. Linear regression analysis of a plot of the In (fraction bound) versus time yielded a dissociation rate constant. The biotin dissociation rate of anti-CD25 scFvSA was identical to that of recombinant streptavidin (r-SA), indicating fully functional biotin binding.

### Example XIX

### Blood Clearance Rate of Anti-CD25 (Anti-TAC) scFvSA and Interaction With Clearing Agent

Blood clearance studies were conducted in female athymic mice (nu/nu; n=3/group) to examine the potential of the fusion protein in pretargeted RIT and to compare it with the humanized mAb (HAT) and HAT/SA chemical conjugate. ¹²⁵I-labeled anti-CD25 scFvSA had a blood clearance half-life (t_{1/2β}) of 11 hours, which was faster than the half lives of the HAT/SA chemical conjugate or HAT mAb (62 hours and 211 hours, respectively). Anti-CD25 scFvSA in circulation is expected to complex efficiently with the synthetic clearing agent (sCA) and be removed rapidly from circulation by subsequent uptake into the liver via the Ashwell receptors. Reactivity of anti-CD25 scFvSA with sCA was assessed in female athymic mice (nu/nu; n=5/group) where ¹²⁵I-anti-TAC scFvSA

1. A vector construct for the expression of streptavidin fusion proteins, comprising:
(a) a first nucleic acid sequence encoding genomic streptavidin or a functional variant thereof, said variant comprising at least 90% amino acid identity with the native sequence thereof, wherein said variant retains the ability to bind biotin;
(b) a promoter operatively linked to the first nucleic acid sequence; and
(c) a cloning site for insertion of a second nucleic acid sequence encoding a anti-CD25 antibody or antigen-binding fragment thereof to be fused with streptavidin, interposed between the promoter and the first nucleic acid sequence.

2. The construct of claim 1, wherein said construct further comprises said second nucleic acid sequence inserted at said cloning site.

3. The construct of claim 1, wherein the promoter is the Lac promoter.

4. The construct of claim 1, wherein the promoter is a constitutive promoter.

5. The construct of claim 1, further comprising S. avidinii regulatory sequences interposed between the promoter and the cloning site.

6. The construct of claim 5, wherein the regulatory sequence is a streptavidin regulatory sequence.

7. The construct of claim 1, further comprising a bacterial leader sequence interposed between the regulatory sequences and the cloning site.

8. The construct of claim 7, wherein the leader sequence comprises a signal sequence.

9. The construct of claim 7, wherein the leader sequence comprises a S. avidinii streptavidin signal sequence.

10. The construct of claim 9, wherein the signal sequence comprises nucleotides 55 to 120 of SEQ ID NO: 88.

11. The construct of claim 1, further comprising a nucleic acid sequence that encodes a protein that is a selectable marker.

12. The construct of claim 11, wherein the protein confers antibiotic resistance.

13. The construct of claim 1, wherein the first nucleic acid sequence encodes at least amino acids 38 to 174 of streptavidin, as set forth in SEQ ID NO: 2.

14. The construct of claim 1, wherein the first nucleic acid sequence encodes at least amino acids selected from the group consisting of 25 to 182, 29 to 182, 38 to 174, 38 to 175, 38 to 176, 38 to 177, 38 to 178, 38 to 179, 38 to 180, 38 to 181, or 38 to 182 of streptavidin, as set forth in SEQ ID NO: 2.

15. A host cell transfected with the construct of claim 1.

16. The host cell of claim 15, wherein the cell is selected from the group consisting of a bacterium, an insect cell, a plant cell, and a mammalian cell.

17. A fusion protein, comprising at least a first and a second polypeptide joined end to end, wherein said first polypeptide comprises at least 129 amino acids of streptavidin, as set forth in SEQ ID NO: 2, or functional variants, said variants comprising at least 90% amino acid identity with the native sequences thereof, wherein said variants retain the ability to bind biotin, and wherein said second polypeptide comprises anti-CD25 antibody or antigen-binding fragment thereof.

18. The fusion protein of claim 17, wherein said first and second polypeptides are separated by a linker of at least two amino acids.

19. The fusion protein of claim 18, wherein the linker is at least four amino acids.

20. The fusion protein of claim 19, wherein the linker consists of four, five, six, seven, eight, nine, ten, eleven, twelve, thirteen, fourteen, fifteen, sixteen, seventeen, eighteen, nineteen, or twenty amino acids.

21. The fusion protein of claim 20, wherein the linker is between five and ten amino acids.

22. The fusion protein of claim 17, wherein the anti-CD25 antibody or antibody fragment thereof comprises a single-chain Fv.

23. The fusion protein of claim 17, wherein the antibody is a humanized antibody.

24. The fusion protein of claim 17, wherein the antibody is a murine antibody.

25. The fusion protein of claim 17, wherein said first polypeptide comprises at least amino acids 38 to 174 of streptavidin, as set forth in SEQ ID NO: 2.

26. The fusion protein of claim 17, wherein the first polypeptide comprises at least amino acids selected from the group consisting of 25 to 182, 29 to 182, 38 to 174, 38 to 175, 38 to 176, 38 to 177, 38 to 178, 38 to 179, 38 to 180, 38 to 181, or 38 to 182 of streptavidin, as set forth in SEQ ID NO: 2.

27. A method for targeting a tumor cell comprising the administration of a fusion protein and a sensitizing agent, wherein said fusion protein comprises at least a first and a second polypeptide joined end to end, wherein said first polypeptide comprises at least 129 amino acids of streptavidin, as set forth in SEQ ID NO: 2, or conservatively substituted variants thereof, wherein said second polypeptide is a targeting agent that binds a cell surface protein, or a cell-associated stromal or matrix protein, on a tumor cell, wherein the streptavidin portion of the fusion protein is capable of binding biotin, and wherein said sensitizing agent is a radiation-sensitizing agent.

28. The method of claim 27, wherein the radiation-sensitizing agent is selected from the group consisting of Gemcitabine, 5-fluorouracil and paclitaxel.

29. The method of claim 28, wherein the radiation-sensitizing agent is Gemcitabine.

30. The method of claim 29, wherein the radiation-sensitizing agent is administered concurrently with administration of a fusion protein.

31. The method of claim 29, wherein the radiation-sensitizing agent is administered prior to administering a fusion protein.

32. The method of claim 29, wherein the radiation-sensitizing agent is administered at a plurality of time points.

33. The method of claim 32, wherein the radiation-sensitizing agent is
(a) administered at a first time prior to administration of the fusion protein, and
(b) at a second time concurrently with administration of a fusion protein.

34. The method of claim 27, wherein the fusion protein binds a cell surface protein receptor, or a cell-associated stromal or matrix protein, on a tumor cell and a biotinylated radionuclide containing compound.

35. The method of claim 27, wherein said first and second polypeptides are separated by a linker of at least two amino acids.

36. The method of claim 35, wherein the linker is at least four amino acids.

37. The method of claim 36, wherein the linker consists of four, five, six, seven, eight, nine, ten, eleven, twelve, thirteen, fourteen, fifteen, sixteen, seventeen, eighteen, nineteen, or twenty amino acids.

38. The method of claim 37, wherein the linker is five to ten amino acids.

39. The method of claim 27, wherein the second polypeptide is an antibody.

40. The method of claim 39, wherein the antibody is B9E9.

41. The method of claim 39, wherein the antibody is CC49.

42. The method of claim 39, wherein the antibody is anti-CD25.

43. The method of claim 27, wherein the antibody is a single-chain Fv fragment.

44. The method of claim 43, wherein the single-chain Fv fragment is derived from antibody anti-CD25.

45. The method of claim 27, wherein the binding agent is an antibody that specifically binds to a cell surface protein, or a cell-associated stromal or matrix protein, selected from the group consisting of CD20, CD22, CD25, CD45, CD52, CD56, CD57, EGP40, CEA, TAG-72, NCAM, β-HCG, a mucin, EGF receptor, IL-2 receptor, her2/neu, Lewis y, GD2, GM2, tenascin, sialylated tenascin, somatostatin, activated tumor stromal antigen, and neoangiogenic antigens.

46. The method of claim 45, wherein the antibody specifically binds CD20

47. The method of claim 45, wherein the antibody specifically binds TAG-72.

48. The method of claim 45, wherein the antibody specifically binds CD25.

49. The method of claim 27, wherein the antibody is a humanized antibody.

50. The method of claim 27, wherein the antibody is a mouse antibody.

51. The method of claim 27, wherein said first polypeptide comprises at least amino acids 38 to 182 of streptavidin, as set forth in SEQ ID NO: 2.

52. The method of claim 27, wherein said first polypeptide comprises at least amino acids 29 to 182 of streptavidin, as set forth in SEQ ID NO: 2.

53. The method of claim 27, wherein said first polypeptide comprises at least amino acids 25 to 182 of streptavidin, as set forth in SEQ ID NO: 2.

54. The method of claim 27, wherein the tumor cell is associated with a cancer selected from the group consisting of carcinomas, adenocarcinomas and hematological malignancies.

55. The method of claim 54, wherein the carcinoma or adenocarcinoma is selected from the group consisting of gliomas, prostate, ovarian, breast, colon, rectal, esophagus, endometrium, appendix, liver, salivary duct, pancreatic, gastric, and lung.

56. The method of claim 54, wherein the hematological malignancy is selected from the group consisting of non-Hodgkin's lymphoma, Hodgkin's disease, peripheral T-cell lymphoma, stages Ib through IV of cutaneous T-cell lymphoma, HTLY-1-associated adult T-cell leukemia, follicular lymphoma, mantle cell lymphoma, diffuse large B-cell lymphoma, precursor B-lymphoblastic lymphoma, lymphoplasmacytoid lymphoma, marginal zone B-cell lymphoma, splenic marginal zone lymphoma, Burkitt's lymphoma, high-grade B cell lymphoma, B-cell chronic lymphocytic lymphoma, small lymphocytic lymphoma, plasmacytoma, melanoma, acute lymphocytic leukemia, prolymphocytic leukemia, precursor B-lymphoblastic leukemia, hairy cell leukemia, acute myelogenous leukemia, chronic lymphocytic leukemia, chronic myelogenous leukemia, multiple myeloma, and Waldenstrom's macroblobulinemia.

57. A method for targeting a tumor cell comprising the administration of a fusion protein, further comprising a sensitizing agent, wherein said fusion protein comprises at least a first and a second polypeptide joined end to end, wherein said first polypeptide comprises at least 129 amino acids of streptavidin, as set forth in SEQ ID NO: 2, or conservatively substituted variants thereof, wherein said second polypeptide is an anti-CD25 antibody or antigen binding fragment thereof, wherein the streptavidin portion of the fusion protein is capable of binding biotin, and wherein said sensitizing agent is a radiation-sensitizing agent.

58. The method of claim 57, wherein the radiation-sensitizing agent is selected from the group consisting of Gemcitabine, 5-fluorouracil and paclitaxel.

59. The method of claim 58, wherein the radiation-sensitizing agent is Gemcitabine.

60. The method of claim 59, wherein the radiation-sensitizing agent is administered concurrently with administration of a fusion protein.

61. The method of claim 59, wherein the radiation-sensitizing agent is administered prior to administering a fusion protein.

62. The method of claim 59, wherein the radiation-sensitizing agent is administered at a plurality of time points.

63. The method of claim 62, wherein the radiation-sensitizing agent is administered prior to administration of the fusion protein and concurrently with administration of a fusion protein.

64. The method of claim 63, wherein the radiation-sensitizing agent is
(a) administered at a first time prior to administration of the fusion protein, and
(b) at a second time concurrently with administration of a fusion protein.

65. The method of claim 43, wherein the single chain antibody comprises variable light and variable heavy chains.

66. The method of claim 65, wherein a linker connects the variable light and variable heavy chains of the single-chain antibody.

67. The method of claim 66, wherein the linker comprises at least ten amino acid residues.

68. The method of claim 67, wherein the linker comprises at least fifteen amino acids.

69. The method of claim 68, wherein the linker comprises at least twenty amino acids.

70. The method of claim 69, wherein the linker comprises at least four repeats of SEQ ID NO: 47.

71. A composition, comprising the fusion protein of any one of claims 17-26 and a physiologically acceptable carrier.

72. A composition comprising a fusion protein comprising a first and a second polypeptide joined end to end, wherein said first polypeptide comprises at least 129 amino acids of streptavidin, as set forth in SEQ ID NO: 2, or functional variants comprising at least 90% amino acid identity with the native sequences thereof, wherein said variants retain the ability to bind biotin, and wherein said second polypeptide comprises a polypeptide that specifically binds a cell surface protein, or a cell-associated stromal or matrix protein, and a radiation-sensitizing agent.

73. The composition of claim 72, wherein said radiation-sensitizing agent is selected from the group consisting of Gemcitabine, 5-fluorouracil and paclitaxel.

74. The composition of claim 73, wherein the radiation-sensitizing agent is Gemcitabine.

75. The composition of claim 72, wherein said second polypeptide is an anti-CD25 antibody or antigen binding fragment thereof.

76. The composition of claim 72, wherein said second polypeptide is an anti-TAG72 antibody or antigen binding fragment thereof.

77. The composition of claim 72, wherein said second polypeptide is an anti-CD20 antibody or antigen binding fragment thereof.

78. A fusion protein, comprising:
(a) a first polypeptide comprising at least 129 amino acids of streptavidin, as set forth in SEQ ID NO: 2, or a functional variant, said variant comprising at least 90% amino acid identity with the native sequence thereof, wherein said variant retains the ability to bind biotin; and
(b) a second polypeptide comprising an antibody, or a fragment thereof, that specifically binds CD25.

79. A fusion protein of claim 78, wherein said antibody is SEQ ID NO: 88.

80. The method of claim 27, wherein the antibody is a rat antibody.


SEQUENCE LISTING (92 sequences)

SEQ ID NO: 1 - DNA, 638 bp, Streptomyces avidinii
ccctccgtccccgccgggcaacaactagggagtatttttcgtgtctcacatgcgcaagatcgtcgttgcagccatcgccgtttccctgaccacggtctcgattacggccagcgcttcggcagacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcagtagtcgcgtcccggcaccggcgggtgccgggacctcggcc

SEQ ID NO: 2 - protein, 183 aa, Streptomyces avidinii
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 3 - DNA, 1614 bp, Artificial Sequence
  huNR-LU-10 single chain antibody-genomic streptavidin fusion.
gaattcacgaagtaaccgacaggactcggccattctttggccgaaattcctttgcagaaaatgttgttgagaaccctccgatggctagtacgatttacaccgaacatgtgcccttggcaaccatcgacccggacctcgaccatccagttctgccgccaaagacacatgccgcactgctgtttgttcaccgacaccgtcaggtgcacggccgaggtcacaaaccttgacgggcgggatacggacggcgcacgccacagcgcgccctccgtcccccgccgggcaacaactaggggagtatttttcgtgtctcacatgcgcaagatcgtcgttgcagccatcgccgtttccctgaccacggtctcgattacggccatggctgacatccagatgactcagtctccatcgtccttgtctgcctctgtgggagacagagtcacgatcacttgtcgggctagtcagggcattagaggtaatttagactggtatcagcagaaacctggtaagggaccgaaactcctaatctactccacatccaatttaaattctggtgtcccatcaaggttcagtggcagtgggtctgggtcagattatactctcaccatcagcagccttcagcctgaagatttcgcaacgtattactgtctacagcgtaatgcgtatccgtacacgttcggacaagggaccaagctggagatcaagatctctggtggcggtggctcgggcggtggtgggtcgggtggcggaggctcgagccaggttcagctggtccagtctggggcagaggtgaaaaagccaggggcctcagtcaaggtgtcctgcaaggcttctggcttcaacattaaagacacctatatgcactgggtgaggcaggcacctggacagggcctgcagtggatgggaaggattgatcctgcgaatggtaatactaaatccgacctgtccttccagggcagggtgactataacagcagacacgtccatcaacacagcctacatggaactcagcagcctgaggtctgacgacactgccgtctattactgttctagagaggtcctaactgggacgtggtctttggactactggggtcaaggaaccttagtcaccgtgagctctggctctggttcggcagacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcagtaaggatcc

SEQ ID NO: 4 - protein, 431 aa, Artificial Sequence
  Predicted amino acid sequence for huNR-LU-10 single chain antibody-genomic streptavidin fusion.
MRKIVVAAIAVSLTTVSITAMADIQMTQSPSSLSASVGDRVTITCRASQGIRGNLDWYQQKPGKGPKLLIYSTSNLNSGVPSRFSGSGSGSDYTLTISSLQPEDFATYYCLQRNAYPYTFGQGTKLEIKISGGGGSGGGGSGGGGSSQVQLVQSGAEVKKPGASVKVSCKASGFNIKDTYMHWVRQAPGQGLQWMGRIDPANGNTKSDLSFQGRVTITADTSINTAYMELSSLRSDDTAVYYCSREVLTGTWSLDYWGQGTLVTVSSGSGSADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 5 - DNA, 1239 bp, Artificial Sequence
  B9E9 single chain antibody-genomic streptavidin fusion
gacatcgtgctgtcgcagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggattagtaacccacccacgttcggtgctgggaccaagctggagctgaagatctctggtctggaaggcagcccggaagcaggtctgtctccggacgcaggttccggctcgagccaggttcagctggtccagtcaggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggtctattactgtgcaagagcgcaattacgacctaactactggtacttcgatgtctggggcgcagggaccacggtcaccgtgagctctggctctggttcggcagacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcagtaa

SEQ ID NO: 6 - protein, 412 aa, Artificial Sequence
  Predicted amino acid sequence of B9E9 single chain antibody-genomic streptavidin fusion
DIVLSQSPAILSASPGEKVTMTCRASSSVSYMHWYQQKPGSSPKPWIYATSNLASGVPARFSGSGSGTSYSLTISRVEAEDAATYYCQQWISNPPTFGAGTKLELKISGLEGSPEAGLSPDAGSGSSQVQLVQSGAELVKPGASVKMSCKASGYTFTSYNMHWVKQTPGQGLEWIGAIYPGNGDTSYNQKFKGKATLTADKSSSTAYMQLSSLTSEDSAVYYCARAQLRPNYWYFDVWGAGTTVTVSSGSGSADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 7 - DNA, 1280 bp, Artificial Sequence
  B9E9 single chain antibody-genomic streptavidin fusion construct  encoding VH-linker-VL-linker-Strept avidin
ccatggctcaggttcagctggtccagtcaggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggtctattactgtgcaagagcgcaattacgacctaactactggtacttcgatgtctggggcgcagggaccacggtcaccgtgagcaagatctctggtggcggtggctcgggcggtggtgggtcgggtggcggcggctcgggtggtggtgggtcgggcggcggcggctcgagcgacatcgtgctgtcgcagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggattagtaacccacccacgttcggtgctgggaccaagctggagctgaagagctctggctctggttcggcagacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcagtaaggatcc

SEQ ID NO: 8 - protein, 423 aa, Artificial Sequence
  Predicted amino acid sequence of B9E9 single chain antibody- genomic streptavidin fusion construct encoding VH-linker-VL-linker- Streptavidin
MAQVQLVQSGAELVKPGASVKMSCKASGYTFTSYNMHWVKQTPGQGLEWIGAIYPGNGDTSYNQKFKGKATLTADKSSSTAYMQLSSLTSEDSAVYYCARAQLRPNYWYFDVWGAGTTVTVSKISGGGGSGGGGSGGGGSGGGGSGGGGSSDIVLSQSPAILSASPGEKVTMTCRASSSVSYMHWYQQKPGSSPKPWIYATSNLASGVPARFSGSGSGTSYSLTISRVEAEDAATYYCQQWISNPPTFGAGTKLELKSSGSGSADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 9 - protein, 18 aa, Artificial Sequence
  pKOD linker
GLEGSPEAGLSPDAGSGS

SEQ ID NO: 10 - protein, 15 aa, Artificial Sequence
  Linker used to create a scFvSA version of anti-CD20mAb, B9E9 in the VLVH orientation
GGGGSGGGGSGGGGS

SEQ ID NO: 11 - protein, 25 aa, Artificial Sequence
  Linker used to create a  version of B9E9 scFvSA in the VHVL orientation
GGGGSGGGGSGGGGSGGGGSGGGGS

SEQ ID NO: 12 - DNA, 32 bp, Artificial Sequence
  Oligonucleotide primer
tgccgtgaattcgtsmarctgcagsartcwgg

SEQ ID NO: 13 - DNA, 31 bp, Artificial Sequence
  Oligonucleotide primer
tgccgtgaattccattswgctgaccartctc

SEQ ID NO: 14 - DNA, 35 bp, Artificial Sequence
  Oligonucleotide primer
tagctggcggccgccctgttgaagctcttgacaat

SEQ ID NO: 15 - DNA, 34 bp, Artificial Sequence
  Oligonucleotide primer
tagctggcggccgctttcttgtccaccttggtgc

SEQ ID NO: 16 - DNA, 47 bp, Artificial Sequence
  Oligonucleotide primer
ttacggccatggctgacatcgtgctgcagtctccagcaatcctgtct

SEQ ID NO: 17 - DNA, 32 bp, Artificial Sequence
  Oligonucleotide primer
caccagagatcttcagctccagcttggtccca

SEQ ID NO: 18 - DNA, 52 bp, Artificial Sequence
  Oligonucleotide primer
cggaggctcgagccaggttcagctggtccagtcaggggctgagctggtgaag

SEQ ID NO: 19 - DNA, 38 bp, Artificial Sequence
  Oligonucleotide primer
gagccagagctcacggtgaccgtggtccctgcgcccca

SEQ ID NO: 20 - DNA, 58 bp, Artificial Sequence
  Oligonucleotide primer
gatctctggtctggaaggcagcccggaagcaggtctgtctccggacgcaggttccggc

SEQ ID NO: 21 - DNA, 58 bp, Artificial Sequence
  Oligonucleotide primer
tcgagccggaacctgcgtccggagacagacctgcttccgggctgccttccagaccaga

SEQ ID NO: 22 - DNA, 50 bp, Artificial Sequence
  Oligonucleotide primer
ttacggccatggctgacatcgtgctgtcgcagtctccagcaatcctgtct

SEQ ID NO: 23 - DNA, 37 bp, Artificial Sequence
  Oligonucleotide primer
ttccggctcgagcgacatcgtgctgtcgcagtctcca

SEQ ID NO: 24 - DNA, 32 bp, Artificial Sequence
  Oligonucleotide primer
gagccagagctcttcagctccagcttggtccc

SEQ ID NO: 25 - DNA, 35 bp, Artificial Sequence
  Oligonucleotide primer
ttacggccatggctcaggttcagctggtccagtca

SEQ ID NO: 26 - DNA, 35 bp, Artificial Sequence
  Oligonucleotide primer
agaccagagatcttgctcacggtgaccgtggtccc

SEQ ID NO: 27 - DNA, 79 bp, Artificial Sequence
  Oligonucleotide primer
gatctctggtggcggtggctcgggcggtggtgggtcgggtggcggcggctcgggtggtggtgggtcgggcggcggcggc

SEQ ID NO: 28 - DNA, 79 bp, Artificial Sequence
  Oligonucleotide primer
tcgagccgccgccgcccgacccaccaccacccgagccgccgccacccgacccaccaccgcccgagccaccgccaccaga

SEQ ID NO: 29 - protein, 18 aa, Artificial Sequence
  Linker sequence
GGGGSGGGGSGGGGSGGS

SEQ ID NO: 30 - protein, 35 aa, Artificial Sequence
  Linker sequence
GGGGSGGGGSGGGGSGGGGSGGGGSGGGGSGGGGS

SEQ ID NO: 31 - protein, 18 aa, Artificial Sequence
  Linker sequence pKOD2
GLEGSPEAGLSPDAGSDS

SEQ ID NO: 32 - DNA, 21 bp, Artificial Sequence
  Oligonucleotide primer
acgacggttgctgcggcggtc

SEQ ID NO: 33 - DNA, 21 bp, Artificial Sequence
  Oligonucleotide primer
aggctcattaatgatgcgggt

SEQ ID NO: 34 - DNA, 33 bp, Artificial Sequence
  Oligonucleotide primer
ggatccaagcttacgatcacggtcatgaacacg

SEQ ID NO: 35 - DNA, 33 bp, Artificial Sequence
  Oligonucleotide primer
ctcgagaagctttaactaaattaatacagcgga

SEQ ID NO: 36 - DNA, 783 bp, Artificial Sequence
  T84.66 single chain antibody-genomic Streptavidin fusion construct
gaggttcagctgcagcagtccggggcagagcttgtggagccaggggcctcagtcaagttgtcctgcacagcttctggcttcaacattaaagacacctatatgcactgggtgaagcagaggcctgaacagggcctggaatggattggaaggattgatcctgcgaatggtaatagtaaatatgtcccgaagttccagggcaaggccactataacagcagacacatcctccaacacagcctacctgcagctcaccagcctgacatctgaggacactgccgtctattattgtgctccgtttggttactacgtgtctgactatgctatggcctactggggtcaaggaacctcagtcaccgtctcctcaaagatctctggtggcggtggctcgggcggtggtgggtcgggtggcggcggctcgggtggtggtgggtcgggcggcggcggctcgagcgacattgtgctgacccaatctccagcttctttggctgtgtctcttgggcagagggccactatgtcctgcagagccggtgaaagtgttgatatttttggcgttgggtttttgcactggtaccagcagaaaccaggacagccacccaaactcctcatctatcgtgcatccaacctagaatctgggatccctgtcaggttcagtggcactgggtctaggacagacttcaccctcatcattgatcctgtggaggctgatgatgttgccacctattactgtcagcaaactaatgaggatccgtacacgttcggaggggggaccaagctggaaataaag

SEQ ID NO: 37 - DNA, 786 bp, Artificial Sequence
  Col-1 single chain antibody-genomic Streptavidin fusion construct
gaagttcagctgcagcagtctggggcagaacttgtgcgttcaggggcctcagtcaaaatgtcctgcaccgcttctggcttcaacattaaagattactatatgcattgggtgaaacagcgtccggaacagggcctggaatggattggttggattgatccggaaaatggtgataccgaatatgccccgaaattccagggcaaagccacgatgaccaccgatacctcctccaacaccgcctacctgcagctcagcagcctgacctctgaagataccgccgtctattactgtaatacccgtggtctatctaccatgattacgacgcgttggttcttcgatgtctggggcgcagggaccacggtcaccgtctccaagatctctggtggcggtggctcgggcggtggtgggtcgggtggcggcggctcgggtggtggtgggtcgggcggcggcggctcgagcgatattgtgctgacccagtctccggcttccttaaccgtatctctgggtctgcgtgccaccatctcatgccgtgccagcaaaagtgtcagtgcatctggctatagttatatgcattggtaccaacagcgtccgggtcagccgccgaaactcctcatctatcttgcatccaacctacaatctggtgtcccggcccgtttcagtggcagtgggtctgggaccgatttcaccctcaacatccatccggtggaagaagaagatgctgcaacctattactgtcagcatagtcgtgaacttccgacgttcggtggtggcaccaaactggaaatcaag

SEQ ID NO: 38 - DNA, 771 bp, Artificial Sequence
  PR1A3 single chain antibody-genomic Streptavidin fusion construct
caggtgaagctgcagcagtcaggtccggagttgaagaagccgggtgagaccgtcaagatcagctgcaaggcttctggttataccttcaccgtgtttggtatgaactgggtgaagcaggctccgggcaagggtttaaagtggatgggctggattaacaccaaaactggtgaagcaacctatgttgaagagtttaagggtcgctttgccttctctttggagacctctgccaccactgcctatttgcagatcaacaacctcaaaaatgaggacacggctaaatatttctgtgcacgttgggacttctatgattacgtggaagctatggattactggggccaagggaccacggtcaccgtctccaagatctctggtggcggtggctcgggcggtggtgggtcgggtggcggcggctcgggtggtggtgggtcgggcggcggcggctcgagcgatattgtgatgacccagtctcaacgtttcatgtccacttcagtaggtgatcgtgtcagcgtcacctgcaaagccagtcagaatgtgggtacgaatgttgcctggtatcaacagaaaccgggtcaatccccgaaagcactgatttactcggcatcctaccgttacagtggtgtcccggatcgcttcaccggcagtggttctgggaccgatttcacgctcaccatcagcaatgtacagtctgaagacttggcggagtatttctgtcatcaatattacacctatccgttattcacgttcggctcggggaccaagttggaaatgaag

SEQ ID NO: 39 - DNA, 762 bp, Artificial Sequence
  MFE-23 single chain antibody-genomic Streptavidin fusion construct
caggtgaaactgcagcagtctggtgcagaacttgtgcgttcagggacctcagtcaaattgtcctgcaccgcttctggcttcaacattaaagattcctatatgcattggttgcgtcagggtccggaacagggcctggaatggattggttggattgatccggagaatggtgatactgaatatgcaccgaagttccagggcaaagccacctttactaccgatacctcctccaacaccgcctacctgcagctcagcagcctgacctctgaagatactgccgtctattattgtaatgaagggactccgactggtccgtactactttgattactggggtcaagggaccacggtcaccgtctccaagatctctggtggcggtggctcgggcggtggtgggtcgggtggcggcggctcgggtggtggtgggtcgggcggcggcggctcgagcgaaaatgtgctcacccagtctccggcaatcatgtctgcatctccgggtgagaaagtcaccattacctgcagtgccagctcaagtgtaagttacatgcattggttccagcagaaaccgggtacttctccgaaactctggatttatagcacctccaacctggcttctggtgttccggctcgcttcagtggcagtggttctgggacctcttactctctcaccatcagccgtatggaagctgaagatgctgccacttattactgccagcaacgtagtagttatccgctcacgttcggtgctggcaccaaactggaactgaag

SEQ ID NO: 40 - DNA, 765 bp, Artificial Sequence
  Nrco-2 single chain antibody-genomic Streptavidin fusion construct
caggtccaactacagcagtcagggggagacttagtgaagcctggagggtccctaaaattctcctgtgcagcctctggattccctttcaatcgctatgccatgtcttgggttcgccagactccagagaagaggctggagtgggtcgcattcattagtagtgatggtatcgcctactatgcagacagtgtgaagggccgattcaccatctccagagataatgccaggaacatcctgtacctacaaatgagcagtctgaggtctgaggacacggccatgtattactgtgcaagagtttattactacggtagtagttactttgactactggggccaagggaccacggtcaccgtgagcaagatctctggtggcggtggctcgggcggtggtgggtcgggtggcggcggctcgggtggtggtgggtcgggcggcggcggctcgagcgacatccagatgactcagtctccaaaattcatgcccacatcagtaggagacagggtcagcgtcacctgcaaggccagtcagaatgcgggtactaatgtagcctggtatcaacagaaaccagggcaatctcctaaagcactgatttactcggcatcgtctcggaacagtggagtccctgatcgcttcacaggcagtggatctgggacagatttcactctcaccatcagcaatgtgcagtctgaagacttggcagagtatttctgtcagcaatataacagctatcctctggtcacgttcggtgctgggaccaagctggaaataaag

SEQ ID NO: 41 - DNA, 768 bp, Artificial Sequence
  CC49 single chain antibody-genomic Streptavidin fusion construct
caggttcagttgcagcagtctgatgctgaattggtgaaaccgggtgcttcagtgaaaatttcctgcaaagcttctggctacaccttcaccgatcatgcaattcattgggtgaaacagaacccggaacagggcctggaatggattggttatttctctccgggtaatgatgatttcaaatacaatgaacgtttcaaaggcaaagccacgctgaccgcagataaatcctccagcaccgcctacgtgcagctcaacagcctgacgtctgaagattctgcagtgtatttctgtacgcgttccctgaatatggcctactggggtcaaggtacctcagtcaccgtctccaagatctctggtggcggtggctcgggcggtggtgggtcgggtggcggcggctcgggtggtggtgggtcgggcggcggcggctcgagcgatattgtgatgtcacagtctccgtcctccctaccggtgtcagttggcgaaaaagttaccttgagctgcaaatccagtcagagccttttatatagtggtaatcagaaaaactacttggcctggtaccagcagaaaccgggtcagtctccgaaactgctgatttactgggcatccgctcgtgaatctggggtcccggatcgcttcaccggcagtggttctgggaccgatttcaccctctccatcagcagtgtgaaaaccgaagacctggcagtttattactgtcagcagtattatagctatccgctcacgttcggtgctgggaccaaactggtgctgaag

SEQ ID NO: 42 - DNA, 765 bp, Artificial Sequence
  BrE-3 single chain antibody-genomic Streptavidin fusion construct
gaagtgaaacttgaagagtctggtggtggcttggtgcaaccgggtggctccatgaaactctcttgtgctgcttctggcttcacctttagtgatgcctggatggattgggtccgccagtctccggagaaagggcttgaatgggttgctgaaattcgtaacaaagccaataatcatggtacctattatgatgagtctgtgaaagggcgcttcaccatctcacgtgatgattccaaaagtcgtgtgtacctgcaaatgattagcttacgtgctgaagataccgggctttattactgtaccggggaatttgctaactggggccaggggacgctggtcaccgtctctaagatctctggtggcggtggctcgggcggtggtgggtcgggtggcggcggctcgggtggtggtgggtcgggcggcggcggctcgagcgatgttgtgatgacccaaactccgctctccctgccggtcactcttggtgatcaagcttccatctcttgccgttctagtcagaaccttgtacataacaatggtaacacctatttatattggttcctgcagaaatcaggccagtctccgaaactgctgatttatcgcgcatccatccgcttttctggtgtcccggatcgcttcagtggcagtggttcagaaaccgatttcacgctcaagatcagccgtgtggaagctgaagacctgggtgtttatttctgctttcaaggtacgcatgttccgtggacgttcggtggtggcaccaaactggaaatcaag

SEQ ID NO: 43 - DNA, 741 bp, Artificial Sequence
  ICR12 single chain antibody-genomic Streptavidin fusion construct
caggtgcagcttcaggagtcaggacctggccttgtgaaaccctcacagtcactctccctcacctgttccgtcactggttactccatcactactgattactggggctggatccggaagttcccaggaaataaaatggagtggatgggatacataagctacagtggtagcactggctacaacccatctctcaaaagtcgaatctccattactagagacacatcgaagagtcagttcttcctgcagttgaactctgtaactactgaggacacagccacatattactgtgcaagatacagtagccttgattactggggccgaggagtcatggtcgcagtctccaagatctctggtggcggtggctcgggcggtggtgggtcgggtggcggcggctcgggtggtggtgggtcgggcggcggcggctcgagcgatgttgtgatgacccagacaccaccgtctttgtcggttgccattggacaatcagtctccatctcttgcaagtcaagtcagagcctcgtatatagtgatggaaagacatatttgcattggttattacagagtcctggcaggtctccgaagcgcctaatctatcaggtgtctaatctgggctctggagtccctgacaggttcagtggcactggatcacagaaagattttacacttaaaatcagcagagtggaggctgaggatttgggagtttactactgcgcgcaaactacacattttcctctcacgttcggttcg

SEQ ID NO: 44 - DNA, 765 bp, Artificial Sequence
  B9E9 single chain antibody-genomic Streptavidin fusion construct
caggttcagctggtccagtcaggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggtctattactgtgcaagagcgcaattacgacctaactactggtacttcgatgtctggggcgcagggaccacggtcaccgtgagcaagatctctggtggcggtggctcgggcggtggtgggtcgggtggcggcggctcgggtggtggtgggtcgggcggcggcggctcgagcgacatcgtgctgtcgcagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggattagtaacccacccacgttcggtgctgggaccaagctggagctgaag

SEQ ID NO: 45 - DNA, 765 bp, Artificial Sequence
  C2B8 single chain antibody-genomic Streptavidin fusion construct
caggttcagctgcaacagccaggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggtctattactgtgcaagaagcacctattacggcggtgattggtacttcaacgtctggggcgcagggaccacggtcaccgtgagcaagatctctggtggcggtggctcgggcggtggtgggtcgggtggcggcggctcgggtggtggtgggtcgggcggcggcggctcgagccagatcgtgctgtcgcagtctccagcaatcctgtctgcatctccaggggagaaggtcacaatgacttgcagggccagctcaagtgtaagttacattcactggtttcagcagaagccaggatcctcccccaaaccctggatttatgccacatccaacctggcttctggagtccctgtgcgcttcagtggcagtgggtctgggacctcttactctctcacaatcagcagagtggaggctgaagatgctgccacttattactgccagcagtggaccagtaacccacccacgttcggtggcgggaccaagctggagatcaag

SEQ ID NO: 46 - DNA, 780 bp, Artificial Sequence
  BC8 single chain antibody-genomic Streptavidin fusion construct
caggttcagctggtggaatcaggaggtggcctggtgcagcctggaggatccctgaaactctcctgtgcagcctcaggattcgatttcagtagatactggatgagttgggtccggcaggctccagggaaagggctagaatggattggagagattaatccaactagcagtacgataaactttacgccatctctaaaggataaagtcttcatctccagagacaacgccaaaaatacgctgtacctgcaaatgagcaaagtgagatccgaggacacagccctttattactgtgcaagagggaactactataggtacggagatgctatggactactggggtcaaggaacctcagtcaccgtgagcaagatctctggtggcggtggctcgggcggtggtgggtcgggtggcggcggctcgggtggtggtgggtcgggcggcggcggctcgagcgacatcgtgctgacccagtctcctgcttccttagctgtatctctgggacagagggccaccatctcatgcagggccagcaaaagtgtcagtacatctggctatagttatctgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccattcacgttcggctcggggacaaagttggaaataaag

SEQ ID NO: 47 - protein, 5 aa, Artificial Sequence
  Gly Ser linker
GGGGS

SEQ ID NO: 48 - DNA, 1467 bp, Artificial Sequence
  CC49 single chain antibody-genomic streptavidin fusion sequence
cacagcgcgccctccgtcccccgccgggcaacaactaggggagtatttttcgtgtctcacatgcgcaagatcgtcgttgcagccatcgccgtttccctgaccacggtctcgattacggccatggctcaggttcagttgcagcagtctgatgctgaattggtgaaaccgggtgcttcagtgaaaatttcctgcaaagcttctggctacaccttcaccgatcatgcaattcattgggtgaaacagaacccggaacagggcctggaatggattggttatttctctccgggtaatgatgatttcaaatacaatgaacgtttcaaaggcaaagccacgctgaccgcagataaatcctccagcaccgcctacgtgcagctcaacagcctgacgtctgaagattctgcagtgtatttctgtacgcgttccctgaatatggcctactggggtcaaggtacctcagtcaccgtctccaagatctctggtggcggtggctcgggcggtggtgggtcgggtggcggcggctcgggtggtggtgggtcgggcggcggcggctcgagcgatattgtgatgtcacagtctccgtcctccctaccggtgtcagttggcgaaaaagttaccttgagctgcaaatccagtcagagccttttatatagtggtaatcagaaaaactacttggcctggtaccagcagaaaccgggtcagtctccgaaactgctgatttactgggcatccgctcgtgaatctggggtcccggatcgcttcaccggcagtggttctgggaccgatttcaccctctccatcagcagtgtgaaaaccgaagacctggcagtttattactgtcagcagtattatagctatccgctcacgttcggtgctgggaccaaactggtgctgaagagctctggctctggttcggcagacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcagtaaggatccggctgctaacaaagcccgaaaggaagctgagttggctgctgccaccgctgagcaataactagcata

SEQ ID NO: 49 - protein, 444 aa, Artificial Sequence
  Predicted amino acid sequence for the CC49 single chain antibody-genomic streptavidin fusion sequence
MRKIVVAAIAVSLTTVSITAMAQVQLQQSDAELVKPGASVKISCKASGYTFTDHAIHWVKQNPEQGLEWIGYFSPGNDDFKYNERFKGKATLTADKSSSTAYVQLNSLTSEDSAVYFCTRSLNMAYWGQGTSVTVSKISGGGGSGGGGSGGGGSGGGGSGGGGSSDIVMSQSPSSLPVSVGEKVTLSCKSSQSLLYSGNQKNYLAWYQQKPGQSPKLLIYWASARESGVPDRFTGSGSGTDFTLSISSVKTEDLAVYYCQQYYSYPLTFGAGTKLVLKSSGSGSADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 50 - DNA, 60 bp, Artificial Sequence
  Oligonucleotide primer
caggttcagttgcagcagtctgatgctgaattggtgaaaccgggtgcttcagtgaaaatt

SEQ ID NO: 51 - DNA, 60 bp, Artificial Sequence
  Oligonucleotide primer
tgcatgatcggtgaaggtgtagccagaagctttgcaggaaattttcactgaagcacccgg

SEQ ID NO: 52 - DNA, 60 bp, Artificial Sequence
  Oligonucleotide primer
tacaccttcaccgatcatgcaattcattgggtgaaacagaacccggaacagggcctggaa

SEQ ID NO: 53 - DNA, 60 bp, Artificial Sequence
  Oligonucleotide primer
tttgaaatcatcattacccggagagaaataaccaatccattccaggccctgttccgggtt

SEQ ID NO: 54 - DNA, 60 bp, Artificial Sequence
  Oligonucleotide primer
ccgggtaatgatgatttcaaatacaatgaacgtttcaaaggcaaagccacgctgaccgca

SEQ ID NO: 55 - DNA, 60 bp, Artificial Sequence
  Oligonucleotide primer
gctgttgagctgcacgtaggcggtgctggaggatttatctgcggtcagcgtggctttgcc

SEQ ID NO: 56 - DNA, 60 bp, Artificial Sequence
  Oligonucleotide primer
gcctacgtgcagctcaacagcctgacgtctgaagattctgcagtgtatttctgtacgcgt

SEQ ID NO: 57 - DNA, 60 bp, Artificial Sequence
  Oligonucleotide primer
gactgaggtaccttgaccccagtaggccatattcagggaacgcgtacagaaatacactgc

SEQ ID NO: 58 - DNA, 35 bp, Artificial Sequence
  Oligonucleotide primer
gaattcccatggctcaggttcagttgcagcagtct

SEQ ID NO: 59 - DNA, 43 bp, Artificial Sequence
  Oligonucleotide primer
caccagagatcttggagacggtgactgaggtaccttgacccca

SEQ ID NO: 60 - DNA, 60 bp, Artificial Sequence
  Oligonucleotide primer
gatattgtgatgtcacagtctccgtcctccctaccggtgtcagttggcgaaaaagttacc

SEQ ID NO: 61 - DNA, 60 bp, Artificial Sequence
  Oligonucleotide primer
accactatataaaaggctctgactggatttgcagctcaaggtaactttttcgccaactga

SEQ ID NO: 62 - DNA, 60 bp, Artificial Sequence
  Oligonucleotide primer
cagagccttttatatagtggtaatcagaaaaactacttggcctggtaccagcagaaaccg

SEQ ID NO: 63 - DNA, 60 bp, Artificial Sequence
  Oligonucleotide primer
agcggatgcccagtaaatcagcagtttcggagactgacccggtttctgctggtaccaggc

SEQ ID NO: 64 - DNA, 60 bp, Artificial Sequence
  Oligonucleotide primer
ctgatttactgggcatccgctcgtgaatctggggtcccggatcgcttcaccggcagtggt

SEQ ID NO: 65 - DNA, 60 bp, Artificial Sequence
  Oligonucleotide primer
tttcacactgctgatggagagggtgaaatcggtcccagaaccactgccggtgaagcgatc

SEQ ID NO: 66 - DNA, 60 bp, Artificial Sequence
  Oligonucleotide primer
ctctccatcagcagtgtgaaaaccgaagacctggcagtttattactgtcagcagtattat

SEQ ID NO: 67 - DNA, 60 bp, Artificial Sequence
  Oligonucleotide primer
caccagtttggtcccagcaccgaacgtgagcggatagctataatactgctgacagtaata

SEQ ID NO: 68 - DNA, 34 bp, Artificial Sequence
  Oligonucleotide primer
cggcggctcgagcgatattgtgatgtcacagtct

SEQ ID NO: 69 - DNA, 38 bp, Artificial Sequence
  Oligonucleotide primer
gagccagagctcttcagcaccagtttggtcccagcacc

SEQ ID NO: 70 - DNA, 60 bp, Artificial Sequence
  Nucleotide primer
caggtccagcttcagcagtctggtgctgaactggcgaaaccgggtgcctcagtgaagatg

SEQ ID NO: 71 - DNA, 60 bp, Artificial Sequence
  Nucleotide primer
acggtagctcgtaaaggtgtagccagaagccttgcaggacatcttcactgaggcacccgg

SEQ ID NO: 72 - DNA, 60 bp, Artificial Sequence
  Nucleotide primer
tacacctttacgagctaccgtatgcattgggttaaacagcgcccgggtcaaggtctggaa

SEQ ID NO: 73 - DNA, 60 bp, Artificial Sequence
  Nucleotide primer
ttccgtataaccggtgctcggattaatatagccaatccattccagaccttgacccgggcg

SEQ ID NO: 74 - DNA, 60 bp, Artificial Sequence
  Nucleotide primer
ccgagcaccggttatacggaatacaatcagaagttcaaggataaggccaccttgacggca

SEQ ID NO: 75 - DNA, 60 bp, Artificial Sequence
  Nucleotide primer
caaattgttctcacccagtctccggcaatcatgtctgcatctccgggtgagaaagtcacc

SEQ ID NO: 76 - DNA, 60 bp, Artificial Sequence
  Nucleotide primer
gtgcatgtaacttatacttgagctggcactgcaggttatggtgactttctcacccggaga

SEQ ID NO: 77 - DNA, 60 bp, Artificial Sequence
  Nucleotide primer
tcaagtataagttacatgcactggttccagcagaaaccgggcacgtctccgaaactctgg

SEQ ID NO: 78 - DNA, 60 bp, Artificial Sequence
  Nucleotide primer
agccgggacaccagaagccaggttggacgtggtataaatccagagtttcggagacgtgcc

SEQ ID NO: 79 - DNA, 60 bp, Artificial Sequence
  Nucleotide primer
ctggcttctggtgtcccggctcgcttcagtggcagtggttctgggacctcttactctctc

SEQ ID NO: 80 - DNA, 60 bp, Artificial Sequence
  Nucleotide primer
ataggtggcagcatcttcagcctccatacggctgatcgtgagagagtaagaggtcccaga

SEQ ID NO: 81 - DNA, 60 bp, Artificial Sequence
  Nucleotide primer
gctgaagatgctgccacctattactgccatcaacgcagtacgtacccgctcacgttcggt

SEQ ID NO: 82 - DNA, 44 bp, Artificial Sequence
  Nucleotide primer
ttcagctccagcttggtcccagaaccgaacgtgagcgggtacgt

SEQ ID NO: 83 - DNA, 34 bp, Artificial Sequence
  Nucleotide primer
cggcggctcgagccaaattgttctcacccagtct

SEQ ID NO: 84 - DNA, 32 bp, Artificial Sequence
  Nucleotide primer
ccaccagagctcttcagctccagcttggtccc

SEQ ID NO: 85 - DNA, 406 bp, Mus musculus
atggaaaggcactggatctttctcttcctgttttcagtaactgcaggtgtccactcccaggtccagcttcagcagtctggggctgaactggcaaaacctggggcctcagtgaagatgtcctgcaaggcttctggctacacctttactagctacaggatgcactgggtaaaacagaggcctggacagggtctggaatggattggatatattaatcctagcactgggtatactgaatacaatcagaagttcaaggacaaggccacattgactgcagacaaatcctccagcacagcctacatgcaactgagcagcctgacatttgaggactctgcagtctattactgtgcaagaggggggggggtctttgactactggggccaaggaaccactctcacagtctcctcag

SEQ ID NO: 86 - DNA, 385 bp, Mus musculus
atgcattttcaagtgcagattttcagcttcctgctaatcagtgcctcagtcataatgtccagaggacaaattgttctcacccagtctccagcaatcatgtctgcatctccaggggagaaggtcaccataacctgcagtgccagctcaagtataagttacatgcactggttccagcagaagccaggcacttctcccaaactctggatttataccacatccaacctggcttctggagtccctgctcgcttcagtggcagtggatctgggacctcttactctctcacaatcagccgaatggaggctgaagatgctgccacttattactgccatcaaaggagtacttacccactcacgttcggttctgggaccaagctggagctgaaac

SEQ ID NO: 87 - DNA, 1371 bp, Mus musculus
gcgccctccgtcccccgccgggcaacaactaggggagtatttttcgtgtctcacatgcgcaagatcgtcgttgcagccatcgccgtttccctgaccacggtctcgattacggccatggctcaggtccagcttcagcagtctggtgctgaactggcgaaaccgggtgcctcagtgaagatgtcctgcaaggcttctggctacacctttacgagctaccgtatgcattgggttaaacagcgcccgggtcaaggtctggaatggattggctatattaatccgagcaccggttatacggaatacaatcagaagttcaaggataaggccaccttgacggcagacaaatcctccagcaccgcctacatgcaactgagcagcctgacgtttgaggattctgcagtctattactgtgcacgtggtggcggtgtctttgattactggggccaaggtaccacgctcaccgtctccaagatctctggtggcggtggctcgggcggtggtgggtcgggtggcggcggctcgggtggtggtgggtcgggcggcggcggctcgagccaaattgttctcacccagtctccggcaatcatgtctgcatctccgggtgagaaagtcaccataacctgcagtgccagctcaagtataagttacatgcactggttccagcagaaaccgggcacgtctccgaaactctggatttataccacgtccaacctggcttctggtgtcccggctcgcttcagtggcagtggttctgggacctcttactctctcacgatcagccgtatggaggctgaagatgctgccacctattactgccatcaacgcagtacgtacccgctcacgttcggttctgggaccaagctggagctgaagagctctggctctggttcggcagacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccatcgacgcggcgaagaaggccggcgtcaacaacggcaacccgctcgacgccgttcagcagtaa

SEQ ID NO: 88 - protein, 438 aa, Mus musculus
MRKIVVAAIAVSLTTVSITAMAQVQLQQSGAELAKPGASVKMSCKASGYTFTSYRMHWVKQRPGQGLEWIGYINPSTGYTEYNQKFKDKATLTADKSSSTAYMQLSSLTFEDSAVYYCARGGGVFDYWGQGTTLTVSKISGGGGSGGGGSGGGGSGGGGSGGGGSSQIVLTQSPAIMSASPGEKVTITCSASSSISYMHWFQQKPGTSPKLWIYTTSNLASGVPARFSGSGSGTSYSLTISRMEAEDAATYYCHQRSTYPLTFGSGTKLELKSSGSGSADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 89 - DNA, 35 bp, Artificial Sequence
  Nucleotide primer
gaattcccatggctcaggtccagcttcagcagtct

SEQ ID NO: 90 - DNA, 46 bp, Artificial Sequence
  Nucleotide primer
caccagagatcttggagacggtgagcgtggtaccttggccccagta

SEQ ID NO: 91 - protein, 135 aa, Mus musculus
MERHWIFLFLFSVTAGVHSQVQLQQSGAELAKPGASVKMSCKASGYTFTSYRMHWVKQRPGQGLEWIGYINPSTGYTEYNQKFKDKATLTADKSSSTAYMQLSSLTFEDSAVYYCARGGGVFDYWGQGTTLTVSS

SEQ ID NO: 92 - protein, 128 aa, Mus musculus
MHFQVQIFSFLLISASVIMSRGQIVLTQSPAIMSASPGEKVTITCSASSSISYMHWFQQKPGTSPKLWIYTTSNLASGVPARFSGSGSGTSYSLTISRMEAEDAATYYCHQRSTYPLTFGSGTKLELK